(19) United States
(12) Patent Application Publication
(10) Pub. No.: US 2026/0207606 A1
(43) Pub. Date: Jul. 23, 2026

(54) DOSING SCHEDULE OF A SOLID DISPERSION OF A HER2 INHIBITOR

(71) Applicant: Boehringer Ingelheim International GmbH, Ingelheim am Rhein (DE)

(72) Inventors: Anke BAUM, Moedling (AT); Shirlynn CHEN, Somers, NY (US); Neil GIBSON, Warthausen (DE); Ralf Richard Hermann LOTZ, Schemmerhofen (DE); Ralph NEUMUELLER, Moedling (AT); Behbood SADROLHEFAZI, Oakville, Ontario (CA); Dirk SCHARN, Berlin (DE); Erica B. SCHLESINGER, Sisters, OR (US); Lukas SCHROETER, Mengen (DE); Josep SERRA FIBLA, Barcelona (ES); Daniel Tod SMITHEY, Bend, OR (US); Ute VON WANGENHEIM, Stockdorf (DE); Randy Joseph WALD, Bend, OR (US); Sven WIND, Ingoldingen (DE)

(21) Appl. No.: 19/141,459

(22) PCT Filed: Dec. 19, 2023
(86) PCT No.: PCT/EP2023/086728

§ 371 Date: Jun. 20, 2025

Related U.S. Application Data
(60) Provisional application No. 63/476,715, filed on Dec. 22, 2022.
(60) Provisional application No. 63/476,733, filed on Dec. 22, 2022.

(30) Foreign Application Priority Data
Feb. 14, 2023 (EP) 23156485.7
Feb. 14, 2023 (EP) 23156619.1
Mar. 29, 2023 (EP) 23382299.8
Oct. 19, 2023 (EP) 23383067.8
Dec. 15, 2023 (EP) 23383302.9

(51) Int. Cl. A61K 31/519; A61K 9/20; A61K 9/28; A61K 45/06; A61P 35/00

(52) U.S. Cl. CPC A61K 31/519; A61K 9/2054; A61K 9/28; A61K 45/06; A61P 35/00

(57) ABSTRACT

The present invention relates to a dosing schedule of a solid dispersion of a HER2 inhibitor and a pharmaceutically acceptable dispersion carrier useful in the prevention and/or treatment of cancer, in particular to its doses and/or second or further line administration.

### FIELD OF THE INVENTION

The present invention relates to a dosing schedule of a solid dispersion of the HER2 inhibitor N-{1-[8-({3-methyl-4-[(1-methyl-1H-1,3-benzodiazol-5-yl)oxy]phenyl}amino)-[1,3]diazino[5,4-d]pyrimidin-2-yl]piperidin-4-yl}prop-2-enamide and a pharmaceutically acceptable dispersion carrier, which is useful in the prevention and/or treatment of cancer. In particular, the dosing schedule can be defined by doses and/or second or further line administration of the HER2 inhibitor.

### BACKGROUND

N-{1-[8-({3-methyl-4-[(1-methyl-1H-1,3-benzodiazol-5-yl)oxy]phenyl}amino)-[1,3]-diazino[5,4-d]pyrimidin-2-yl]piperidin-4-yl}prop-2-enamide, also herein referred to as compound (1) or zongertinib, is a HER2 (ErbB2) inhibitor described in WO 2021/213800. Zongertinib is a potent and selective tyrosine kinase inhibitor of wild type and mutant HER2 that spares wild type epithelial growth factor receptor (EGFR). Therefore, it is useful for the treatment and/or prevention of diseases and/or conditions wherein the inhibition of wild type and/or mutant HER2 is of therapeutic benefit, especially oncological and/or hyperproliferative diseases, such as cancer.

The solubility of compound (1) in aqueous media was found to be limited and strongly pH dependent with increased solubility at acidic conditions. Specifically, about 10⁵-fold decrease in solubility was observed between pH 1.2 and pH 6.8. Consequently, the in vivo absorption of compound (1) is affected by gastric pH. In particular, low gastric pH is associated with an increase in absorption of compound (1), whereas an increased gastric pH leads to lower blood serum levels of compound (1). This pH dependency is undesirable because it may decrease bioavailability and/or bioaccessibility of compound (1). Such a decrease may occur to different extents in different patients due to inter-patient stomach pH variability.

Moreover, the gastric pH of cancer patients may be altered due to a therapeutic agent in their treatment plan, such as protein pump inhibitors (PPIs), antacids or antihistamines, which are acid-reducing agents known to increase gastric pH. These therapeutic agents are often administered to cancer patients, for instance to mediate gastrointestinal side effects brought about by medicaments, in particular those that lower gastric pH, but also to manage effects of a tumor in the gastric area. A co-administration of acid reducing agents and compound (1) may thus reduce absorption and systemic exposure of compound (1).

Therefore, there remains a need to decrease the pH dependency of compound (1) to improve its bioavailability and/or bioaccessibility.

In addition, there is the need to find a way of administering compound (1) that is therapeutically efficacious while also being safe and tolerable as such and in the background of already performed treatments.

### BRIEF DESCRIPTION OF THE FIGURES

FIG. 1 shows the x-ray powder diffractograms (XRPDs) of spray dried amorphous solid dispersions of compound (1) with 75 wt % HPMCAS-M (top curve) and 50 wt % HPMCAS-M (bottom curve) obtained from Example 1 herein compared to the XRPD of crystalline compound (1).

FIG. 2 shows the x-ray powder diffractograms (XRPDs) of spray dried amorphous solid dispersions of compound (1) with 75 wt % PVP-VA (top curve) and 50 wt % PVP-VA (bottom curve) obtained from Example 1 herein compared to the XRPD of crystalline compound (1).

FIG. 3 shows the x-ray powder diffractograms (XRPDs) of spray dried amorphous solid dispersions of compound (1) with 75 wt % Eudragit® L100 (top curve) and 50 wt % Eudragit® L100 (bottom curve) obtained from Example 1 herein compared to the XRPD of crystalline compound (1).

FIG. 4 shows the x-ray powder diffractograms (XRPDs) of spray dried amorphous solid dispersions of compound (1) with 75 wt % HPMC HME 15LV (top curve) and 50 wt % HPMC HME 15LV (bottom curve) obtained from Example 1 herein compared to the XRPD of crystalline compound (1).

FIG. 5 shows the x-ray powder diffractograms (XRPDs) of spray dried amorphous solid dispersions of compound (1) obtained from Example 3.3 herein. From top curve to bottom curve: sample 3.3-A (top curve), sample 3.3-B (middle curve) and sample 3.3-C (bottom curve).

FIG. 6 shows the x-ray powder diffractograms (XRPDs) of spray dried amorphous solid dispersions of compound (1) obtained from Example 3.4 herein. From top curve to bottom curve: sample 3.4-A (top curve) and sample 3.4-B (bottom curve).

FIG. 7 shows the x-ray powder diffractograms (XRPDs) of amorphous solid dispersions of compound (1) with HPMCAS-M (50 wt %:50 wt %) after exposure to 75° C./79% relative humidity and 80° C./76% relative humidity for three weeks. From top curve to bottom curve: unstressed sample (top), stressed sample at 75° C./79% relative humidity (middle) and stressed sample at 80° C./76% relative humidity (bottom).

FIG. 8 shows a comparison of Log solubility values determined in aqueous media at different pH: amorphous solid dispersion of compound (1) with HPMCAS-M (50 wt %:50 wt %) (circles), crystalline Form III of compound (1) (squares), and crystalline Form IV of compound (1) (triangles).

FIG. 9 shows in-vitro dissolution profiles of various amorphous solid dispersions (25 wt %:75 wt % compound (1):polymer) comprising different polymers vs the dissolution profile of crystalline compound (1) in simulated intestinal fluid after transfer from simulated gastric fluid in the two-stage gastric transfer test of Example 5.2. From top curve to bottom curve (in relation to the first measuring point): IPMC HME 15LV, PVP-VA, HPMCAS-M, Eudragit® L100 and crystalline compound (1).

FIG. 10 shows in-vitro dissolution profiles of various amorphous solid dispersions (50 wt %:50 wt % compound (1):polymer) comprising different polymers vs the dissolution profile of crystalline compound (1) in simulated intestinal fluid after transfer from simulated gastric fluid in the two-stage gastric transfer test of Example 5.2. From top curve to bottom curve (in relation to the first measuring point): HPMCAS-M, IPMC HME 15LV, PVP-VA, Eudragit® L100 and crystalline compound (1).

FIG. 11 shows results of an in vitro dissolution comparison at pH 2.0 between tablets containing crystalline compound (1) (squares) and a solid dispersion of compound (1) (circles).

FIG. 12 shows results of an in vitro dissolution comparison at pH 6.8 between conventional tablets containing crystalline compound (1) (circles), and tablets containing a solid dispersion of compound (1) (squares Example 6.2-A, triangles Example 6.2-C).

FIG. 13 shows a schematic representation of the dynamic in vitro gastrointestinal model for the simulation of the physiological processes occurring in human stomach and small intestine tiny-TIM model. A: meal inlet; B: corpus; C: proximal antrum; D: distal antrum; E: pyloric valve; F: peristaltic valve; G: small intestinal compartment; H: filtration system; I: gastric secretion; J: intestinal secretion; K: pH electrode; L: level sensor.

FIG. 14 shows results of an in vitro determination of bioaccessibility over time of the solid dispersion of compound (1) (“SDD”) compared to administration of a conventional tablet of crystalline compound (1) (“Conv.”), both at 100 mg dose, under fasted conditions (normally low gastric pH) and simulated higher gastric pH conditions following administration of a proton pump inhibitor (PPI).

FIG. 15 shows the x-ray powder diffractograms (XRPDs) of the formulation disclosed under Example 6.1-C.

FIG. 16 shows the x-ray powder diffractograms (XRPDs) of the formulation disclosed under Example 6.2-C.

FIG. 17 shows the design of the dose escalation part of a clinical trial testing different doses and schedules of compound (1) in pre-treated patients with unresectable, advanced and/or metastatic solid tumors with an aberration of the HER2 gene, as described in Example 1. N=number of patients; BID=bis in die, twice daily; QD=quaque die, once a day; RP2D=dose recommended for dose expansion.

FIG. 18 shows the swimmer plot of response assessments and duration of treatment by patient and dose in the BID schedule as of Mar. 16, 2023. The bars represent progression-free survival durations, not treatment durations. BID=bis in die, twice daily; QD=quaque die, once a day; PR=partial response; SD=stable disease; PD=disease progression.

FIG. 19 shows the swimmer plot of response assessments and duration of treatment by patient and dose in the BID schedule as of Mar. 16, 2023. The bars represent progression-free survival durations, not treatment durations. Abbreviations are defined as in FIG. 2.

FIG. 20 shows the swimmer plot of response assessments and duration of treatment by patient and dose in the QD schedule as of Mar. 16, 2023. The bars represent progression-free survival durations, not treatment durations. Abbreviations are defined as in FIG. 2.

FIG. 21 shows the waterfall plot of BID vs QD schedules showing best change from baseline in target lesions (RECIST v1.1) expressed as a percentage.

FIG. 22 shows the waterfall plot of BID dose levels showing best change from baseline in target lesions (RECIST v1.1) expressed as a percentage.

FIG. 23 shows the waterfall plot of QD dose levels showing best change from baseline in target lesions (RECIST v1.1) expressed as a percentage.

FIG. 24 shows the waterfall plot of BID dose levels showing best change from baseline in target lesions (RECIST v1.1) expressed as a percentage in (a) non-small cell lung cancer patients and (b) patients with other tumors.

FIG. 25 shows the waterfall plot of QD dose levels showing best change from baseline in target lesions (RECIST v1.1) expressed as a percentage in (a) non-small cell lung cancer patients and (b) patients with other tumors.

### SUMMARY

According to a first aspect, it is provided herein a solid dispersion comprising compound (1) as defined below or a pharmaceutically acceptable salt thereof

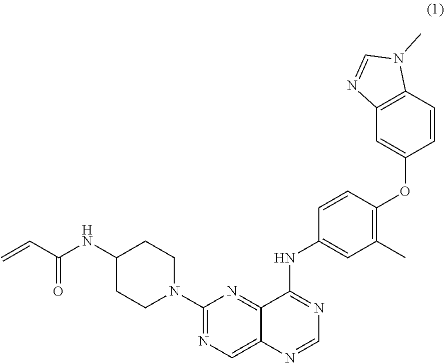 [SMILES: C=CC(=O)NC1CCN(c2ncc3ncnc(Nc4ccc(Oc5ccc6c(c5)ncn6C)c(C)c4)c3n2)CC1]

and a pharmaceutically acceptable dispersion carrier, wherein the solid dispersion is for use in a method for the prevention and/or the treatment of cancer, wherein compound (1) is administered in a daily dose of at least 30 mg. Also provided herein is a method for the prevention and/or treatment of cancer comprising administering to a subject in need thereof a solid dispersion comprising compound (1) as defined herein or a pharmaceutically acceptable salt thereof and a pharmaceutically acceptable dispersion carrier, wherein compound (1) is administered in a daily dose of at least 30 mg.

According to another aspect, it is provided herein a solid dispersion comprising compound (1) as defined below or a pharmaceutically acceptable salt thereof

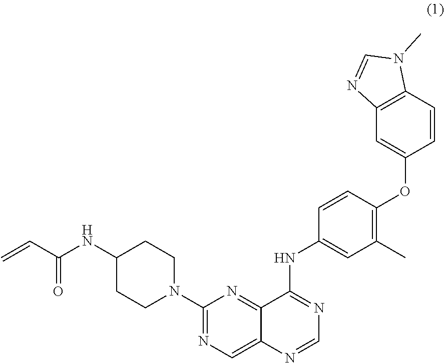 [SMILES: C=CC(=O)NC1CCN(c2ncc3ncnc(Nc4ccc(Oc5ccc6c(c5)ncn6C)c(C)c4)c3n2)CC1]

and a pharmaceutically acceptable dispersion carrier, wherein the solid dispersion is for use in a method for the prevention and/or the treatment of cancer, wherein compound (1) is administered following administration of a systemic anti-cancer therapy agent. Also provided herein is a method for the prevention and/or treatment of cancer comprising administering to a subject in need thereof a solid dispersion comprising compound (1) as defined herein or a pharmaceutically acceptable salt thereof and a pharmaceutically acceptable dispersion carrier, wherein compound (1) is administered following administration of a systemic anti-cancer therapy agent.

In embodiments, the pharmaceutically acceptable dispersion carrier is a polymer.

In embodiments, the polymer is enteric or non-enteric.

In embodiments, the pharmaceutically acceptable dispersion carrier is a polymer selected from the group consisting of hydroxypropyl methylcelluloses and esters thereof, polyvinylpyrrolidones and copolymers thereof, and polymethacrylates and copolymers thereof.

In embodiments, the hydroxypropyl methylcelluloses and esters thereof are selected from the group consisting of hydroxypropyl methylcellulose acetate succinate and hydroxypropyl methylcellulose, in particular hot melt extrusion-grade hydroxypropyl methylcellulose.

In embodiments, the polyvinylpyrrolidones and copolymers thereof are a polyvinylpyrrolidone vinyl acetate copolymer.

In embodiments, the polymethacrylates and copolymers thereof are a methylacrylic acid methyl methacrylate copolymer.

In embodiments, the pharmaceutically acceptable dispersion carrier is a polymer selected from the group of hydroxypropyl methylcellulose acetate succinate, polyvinylpyrrolidone vinyl acetate copolymer, methylacrylic acid methyl methacrylate copolymer, and hot melt extrusion-grade hydroxypropyl methylcellulose.

In embodiments, compound (1) is amorphous.

In embodiments, compound (1) is present in an amount in a range of from 25 wt % to 75 wt %, based on a total weight of 100 wt % of the solid dispersion.

In embodiments, the pharmaceutically acceptable dispersion carrier is present in an amount in a range of from 25 wt % to 75 wt %, based on a total weight of 100 wt % of the solid dispersion.

In embodiments, the weight ratio of compound (1): the pharmaceutically acceptable dispersion carrier in the solid dispersion is of 1:1 to 1:3.

In embodiments, the solid dispersion for use as described herein is characterized by having an x-ray powder diffractogram comprising no diffraction peak at 2-theta angles equal or below 40.0°, when measured at a temperature in the range of from 20 to 30° C. and with Cu-Kα radiation having a wavelength of 1.54056 Å or 1.54184 Å.

Also provided herein is a pharmaceutical composition comprising the solid dispersion as defined herein and one or more pharmaceutically acceptable excipients, wherein the pharmaceutical composition is for use in a method for the prevention and/or the treatment of cancer, wherein compound (1) is administered in a daily dose of at least 30 mg.

In embodiments, the one or more pharmaceutically acceptable excipients are selected from the group consisting of fillers, disintegrants, glidants, lubricants, and coating agents.

In embodiments, the fillers are selected from the group consisting of microcrystalline cellulose, mannitol and mixtures thereof.

In embodiments, the disintegrants are selected from the group consisting of croscarmellose sodium, sodium bicarbonate, crospovidone, sodium starch glycolate and mixtures thereof.

In embodiments, the glidant is colloidal silicon dioxide.

In embodiments, the lubricants are selected from the group consisting of stearyl fumarate, magnesium stearate and mixtures thereof.

In embodiments, the one or more pharmaceutically acceptable excipients comprise mannitol, microcrystalline cellulose, croscarmellose sodium, colloidal silicon dioxide and sodium stearyl fumarate.

In embodiments, the pharmaceutical composition for use as described herein, based on a total weight of 100 wt % of the pharmaceutical composition, comprises:
in a range of from 25 wt % to 65 wt % of the solid dispersion as defined herein; and/or
in a range of from 25 wt % to 65 wt % of one or more fillers; and/or
in a range of from 4 wt % to 10 wt % of disintegrant; and/or
in a range of from 1 wt % to 2 wt % of glidant; and/or
in a range of from 1 wt % to 2 wt % of lubricant; and/or
optionally a range of from 2 wt % to 5 wt % of coating agent.

In embodiments, the pharmaceutical composition for use as described herein is in the form of a tablet, of granules or of a capsule.

In embodiments, the pharmaceutical composition for use as described herein comprises: (i) a tablet core comprising the solid dispersion as defined herein, mannitol, microcrystalline cellulose, croscarmellose sodium, colloidal silicon dioxide and sodium stearyl fumarate; and (ii) a film coating.

In embodiments, the pharmaceutical composition for use as described herein is characterized by having an x-ray powder diffractogram comprising no diffraction peak at 2-theta angles equal or below 6.5°, when measured at a temperature in the range of from 20 to 30° C. and with Cu-Kα radiation having a wavelength of 1.54056 Å or 1.54184 Å.

In embodiments, the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In embodiments, said cancer is a HER2 overexpressed, HER2 amplified and/or HER2 mutant cancer.

In embodiments, said cancer is advanced or metastatic cancer.

In embodiments, the solid dispersion or the pharmaceutical composition is administered to a fasted subject.

In embodiments, the solid dispersion or the pharmaceutical composition is administered in combination with a medicament that increases gastric pH.

In embodiments, the medicament that increases gastric pH is selected from the group consisting of a proton-pump inhibitor, an antacid and an antihistamine.

In embodiments, compound (1) is administered in a daily dose of 30 mg to 600 mg.

In embodiments, compound (1) is administered in a daily dose of 30 mg, 60 mg, 120 mg, 180 mg, 200 mg, 240 mg, 300 mg, 360 mg, 400 mg, 420 mg, 480 mg, 500 mg, 540 mg or 600 mg.

In embodiments, compound (1) is administered once or twice daily.

In embodiments, compound (1) is administered once daily in a daily dose of 60 mg, 120 mg, 180 mg, 240 mg, 300 mg, 360 mg, 400 mg, 420 mg, 480 mg, 500 mg, 540 mg or 600 mg.

In embodiments, compound (1) is administered twice daily in a daily dose of 30 mg, 60 mg, 120 mg, 200 mg, 300 mg, 360 mg, 400 mg, 420 mg, 480 mg, 500 mg, 540 mg or 600 mg. In embodiments, the systemic anti-cancer therapy agent is selected from the group consisting of platinum-based chemotherapy, anti-HER2 antibody-drug conjugates and combinations thereof.

Further embodiments are represented by the following:

1) Compound (1) as defined below

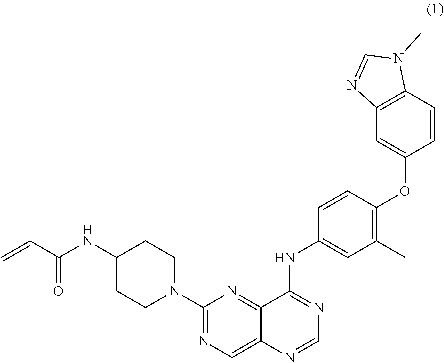 [SMILES: C=CC(=O)NC1CCN(c2ncc3ncnc(Nc4ccc(Oc5ccc6c(c5)ncn6C)c(C)c4)c3n2)CC1]

for use in the treatment of cancer, wherein compound (1) is administered in a daily dose of at least 30 mg.

2) A method for the prevention and/or treatment of cancer comprising administering to a subject in need thereof compound (1) as defined herein in a daily dose of at least 30 mg.

3) The compound for use according to embodiment 1) or the method according to embodiment 2), wherein compound (1) is administered in a daily dose of 30 mg to 600 mg.

4) The compound for use according to embodiment 1) or 3) or the method according to embodiment 2) or 3), wherein compound (1) is administered in a daily dose of 30 mg, 60 mg, 120 mg, 180 mg, 200 mg, 240 mg, 300 mg, 360 mg, 400 mg, 420 mg, 480 mg, 500 mg, 540 mg or 600 mg.

5) The compound for use according to embodiment 1) or 3) to 4) or the method according to embodiment 2) or 3) to 4), wherein compound (1) is administered once or twice daily.

6) The compound for use according to embodiment 1) or 3) to 5) or the method according to embodiment 2) or 3) to 5), wherein compound (1) is administered once daily in a daily dose of 60 mg, 120 mg, 180 mg, 240 mg, 300 mg, 360 mg, 400 mg, 420 mg, 480 mg, 500 mg, 540 mg or 600 mg or compound (1) is administered twice daily in a daily dose of 30 mg, 60 mg, 120 mg, 200 mg, 300 mg, 360 mg, 400 mg, 420 mg, 480 mg, 500 mg, 540 mg or 600 mg.

7) The compound for use according to embodiment 1) or 3) to 6) or the method according to embodiment 2) or 3) to 6), wherein compound (1) is administered orally.

8) The compound for use according to embodiment 1) or 3) to 7) or the method according to embodiment 2) or 3) to 7), wherein compound (1) is administered as a tablet.

9) Compound (1) as defined below

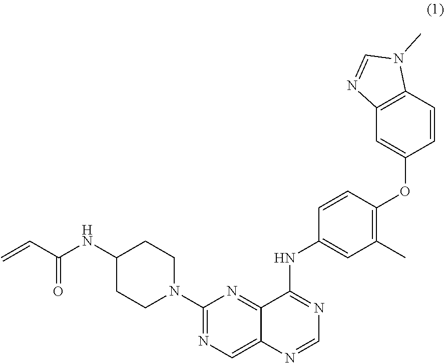 [SMILES: C=CC(=O)NC1CCN(c2ncc3ncnc(Nc4ccc(Oc5ccc6c(c5)ncn6C)c(C)c4)c3n2)CC1]

for use in the treatment of cancer, wherein compound (1) is administered following administration of a systemic anti-cancer therapy agent.

10) A method for the prevention and/or treatment of cancer comprising administering to a subject in need thereof compound (1) following administration of a systemic anti-cancer therapy agent.

11) The compound for use according to embodiment 9) or the method according to embodiment

10), wherein compound (1) is administered as defined in any embodiment herein, especially with respect to embodiments 1) to 8).

12) The compound for use according to embodiment 9) or 11) or the method according to embodiment 10), wherein the systemic anti-cancer therapy agent is selected from the group consisting of platinum-based chemotherapy, anti-HER2 antibody-drug conjugates and combinations thereof.

13) The compound for use according to embodiment 9), 11) or 12) or the method according to any one of embodiments 10) to 12), wherein the cancer is selected from the group consisting of brain cancer, breast cancer, biliary tract cancer, bladder cancer, cervical cancer, uterine cancer, colorectal cancer, endometrial cancer, skin cancer, gastric cancer, esophagus tumor, head and neck tumor, gastrointestinal cancer, gallbladder tumor, kidney cancer, liver cancer, lung cancer and prostate cancer.

14) The compound for use according to embodiment 9) or 11) to 13) or the method according to any one of embodiments 10) to 13), wherein said cancer is HER2 overexpressed, HER2 amplified and/or HER2 mutant.

15) The compound for use according to embodiment 9) or 11) to 14) or the method according to any one of embodiments 10) to 14), wherein said cancer is advanced or metastatic.

16) A pharmaceutical composition comprising compound (1) as defined below

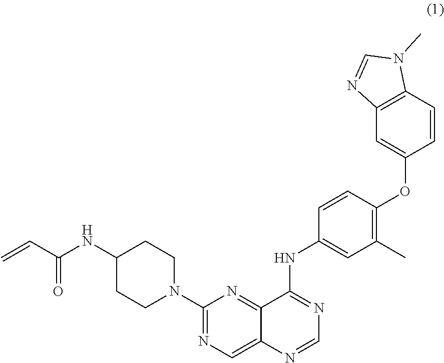 [SMILES: C=CC(=O)NC1CCN(c2ncc3ncnc(Nc4ccc(Oc5ccc6c(c5)ncn6C)c(C)c4)c3n2)CC1]

and at least one pharmaceutically acceptable excipient for use in the treatment of cancer, wherein compound (1) is administered in a daily dose of at least 30 mg.

17) A method for the prevention and/or treatment of cancer comprising administering to a subject in need thereof a pharmaceutical composition comprising compound (1) and at least one pharmaceutically acceptable excipient, wherein compound (1) is administered in a daily dose of at least 30 mg.

18) The pharmaceutical composition according to embodiment 16) or the method according to embodiment 17), wherein compound (1) is administered according to any of embodiments 1) to 15).

All embodiments described herein relative to compound (1) for use in the treatment of cancer or to the method of treating a patient suffering from cancer with compound (1) are applicable to the pharmaceutical composition for use in the treatment of cancer or to the method of treating a patient suffering from cancer with the pharmaceutical composition.

### DETAILED DESCRIPTION

It is a purpose of the present invention to decrease the pH dependency of compound (1) to improve its bioavailability and/or bioaccessibility.

It was surprisingly discovered that a formulation of compound (1) as a solid dispersion has the potential to achieve consistent bioavailability and/or bioaccessibility and to overcome inter-patient stomach pH variability compared to the administration of formulations comprising compound (1) in crystalline form. Therefore, the present invention provides a solid dispersion comprising compound (1) as defined herein or a pharmaceutically acceptable salt thereof and a pharmaceutically acceptable dispersion carrier.

Specifically, the administration of a solid dispersion of compound (1) provides a high up-take not only when administered to a subject with a normally low gastric pH but as well when administered in combination with a medicament that increases gastric pH such as a proton pump inhibitor, an antacid or an antihistamine. The surprising results shown in the examples described herein, in particular the superior in-vitro and in-vivo performance of the solid dispersions of the present invention compared to formulations comprising crystalline compound (1) demonstrated in Examples 5.1, 5.2 and 7.1 to 7.4 herein, indicate that the formulation of compound (1) as a solid dispersion provides a consistently high up-take unaffected by pH variations e.g. initiated by co-medication that causes a rise in stomach pH level and thus results in the possibility of including patient groups under co-medication such as under proton pump inhibitors, antacids or antihistamines in treatment with compound (1).

It was also surprising to find that compound (1) could be kept in the amorphous state such as in the solid dispersion, even when subjected to extended temperature and moisture stress as demonstrated in Example 4 herein.

It is also a purpose of the present invention to provide a safe and efficacious dose regime for compound (1) for use in the treatment of cancer. Surprisingly it has been found that the dose regime according to the invention achieved clinical efficacy, as evidenced by partial responses with an overall response rate (ORR) of 45.8% with a good disease control rate (DCR) of 95.8% (excluding patients that have a best overall response of “non-evaluable” at the time of data cut-off).

At the same time, the side effects brought by the dose regime according to the invention are surprisingly few and mild, as demonstrated by the facts that only 3 dose limiting toxicities (DLT) have been observed outside the maximum tolerated dose (MTD) observation period and the MTD has not been reached yet. This indicates good safety and tolerability of the medical uses and method of treatments according to the invention, as well as a low discontinuation rate.

In addition, the dose regime according to the invention seems also to be suitable for a variety of patients who have already received other cancer related treatments. As used herein, the terms “dose regime” and “dose schedule” are intended as synonyms of “administration regime” and “administration schedule”, thus not necessarily limited to any specific dose, as is the case for some aspects and embodiments concerning second or further lines. Therefore, the terms “dose regime” and “dose schedule” also include administration regimes or schedules defined by a line of administration, but not necessarily by a dose.

The positive safety profile observed with the dosing schedules of the invention allows for relatively high amounts of compound (1) to be administered at the same time. This in turn can have several advantageous effects, for instance on patient compliance.

Dose-efficacy and dose-safety relationships were unexpectedly found to be flat which can be indicative of a broad therapeutic window of the invention.

### Compound (1)

As used herein, the term “compound (1)” refers to the compound as defined below or a pharmaceutically acceptable salt thereof:

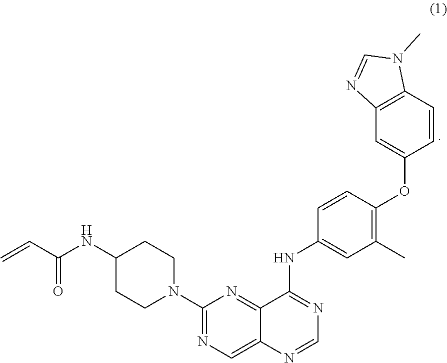 [SMILES: C=CC(=O)NC1CCN(c2ncc3ncnc(Nc4ccc(Oc5ccc6c(c5)ncn6C)c(C)c4)c3n2)CC1]

The IUPAC name of compound (1) is N-{1-[8-({3-methyl-4-[(1-methyl-1H-1,3-benzodiazol-5-yl)oxy]phenyl}amino)-[1,3]diazino[5,4-d]pyrimidin-2-yl]piperidin-4-yl}prop-2-enamide. In case of discrepancy between IUPAC name and depicted formula, the formula shall prevail. Compound (1) is also known as zongertinib. Compound (1) is disclosed in WO 2021/213800 as example compound I-01. WO 2021/213800 describes [1,3]diazino[5,4-d]pyrimidines such as compound (1) as HER2 inhibitors and provides a synthesis procedure for compound (1). Properties of compound (1) and evidence for inhibitory effect on HER2 wild-type and YVMA kinase activity, while sparing EGFR, are also disclosed in WO 2021/213800, which is herein incorporated by reference.

The term “compound (1)” as used herein also encompasses any tautomers and pharmaceutically acceptable salts and all solid state forms of the compound, as well as solvates, including hydrates and solvates of pharmaceutically acceptable salts thereof.

In embodiments, compound (1) is a free base. Therefore, in any aspect or embodiment, the expression “compound (1) or a pharmaceutically acceptable salt thereof” can be replaced by “compound (1)”, without a reference to the pharmaceutically acceptable salt thereof. In embodiments, pharmaceutically acceptable salts of compound (1) are used. The term “pharmaceutically acceptable” used herein refers to compounds, materials, compositions and/or dosage forms which are, within the scope of sound medical judgement, suitable for use in contact with the tissues of human beings without excessive toxicity, irritation, allergic response, or other problem or complication, and commensurate with a reasonable benefit/risk ratio.

As used herein “pharmaceutically acceptable salts” of compound (1) refers to compound (1) wherein the compound is modified by making acid or base salts thereof. The term pharmaceutically acceptable salts as used herein generally includes both acid and base addition salts. Pharmaceutically acceptable acid addition salts refer to those salts which retain the biological effectiveness and properties of the free base and which are not biologically or otherwise undesirable, formed with inorganic acids or organic acids. Pharmaceutically acceptable base addition salts include salts derived from inorganic bases or organic nontoxic bases. Examples of pharmaceutically acceptable salts include, but are not limited to, mineral or organic acid salts of basic residues such as amines; alkali or organic salts of acidic residues such as carboxylic acids; and the like. For example, such salts include salts from benzenesulfonic acid, benzoic acid, citric acid, ethane sulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. In embodiments, pharmaceutically acceptable salts are selected from chloride and fumarate salts.

Pharmaceutically acceptable salts can be synthesized from compound (1) by conventional chemical methods. Generally, such salts can be prepared by reacting the free base form of compound (1) with a sufficient amount of the appropriate acid or base in water or in an organic diluent or solvent like ether, ethyl acetate, ethanol, isopropanol, or acetonitrile, or a mixture thereof.

The term “solvate” as used herein refers to an association or complex of one or more solvent molecules and compound (1). Examples of solvents include water, isopropanol, ethanol, methanol, dimethyl sulfoxide (DMSO), ethyl acetate, acetic acid, tert-butyl methyl ether, tetrahydrofuran, methylethyl ketone, N-methylpyrrolidone and ethanolamine. The term “hydrate” refers to a complex where the solvent molecule is water.

### Solid Dispersion

In embodiments, the solid dispersion as described herein consists essentially of compound (1) as defined herein or a pharmaceutically acceptable salt thereof and a pharmaceutically acceptable dispersion carrier. As used herein, the expressions “consists essentially of” and “consisting essentially of” have the meaning attributed to them in the art. In particular, they indicate that further components may be present, especially those further components that do not have a material effect on the characteristics of the respective dispersion, composition or formulation. Such further components may for example be residual solvents.

In embodiments, the solid dispersion as described herein consists of compound (1) as defined herein or a pharmaceutically acceptable salt thereof and a pharmaceutically acceptable dispersion carrier.

As used herein, the term “solid dispersion” refers to a system in a solid state comprising at least two components, wherein one component, such as compound (1) or generally an active pharmaceutical ingredient (API), preferably in amorphous state, is dispersed throughout another component such as a pharmaceutically acceptable solid dispersion carrier, particularly a dispersion polymer.

As used herein, the term “dispersion carrier” refers to a carrier component that allows for an API such as compound (1) to be dispersed throughout such that a solid dispersion may form. In embodiments, compound (1) is dispersed at the molecular level in the pharmaceutically acceptable dispersion carrier.

In embodiments, the pharmaceutically acceptable dispersion carrier is a polymer. Therefore, the present invention provides a solid dispersion comprising compound (1) or a pharmaceutically acceptable salt thereof, especially for use as defined herein, and a polymer.

Polymeric dispersion carriers also are denoted “dispersion polymers”. Therefore, the present invention provides a solid dispersion comprising compound (1) as defined herein or a pharmaceutically acceptable salt thereof and a polymer, wherein the solid dispersion is for use as defined herein in any aspect or embodiment. Polymers are widely used in solid dispersion formulations. Different polymeric carriers lead to solid dispersions with various properties in terms of physical stability, phase behavior and drug release rate and extent. Due to the complex nature solid dispersion formulation carrier best suited for a given API need to be tested. The pharmaceutically acceptable dispersion polymer preferably is a neutral or acidic polymer. In other embodiments, the pharmaceutically acceptable dispersion carrier is a polymer that is enteric or non-enteric, preferably enteric. In other embodiments, the polymer is enteric or non-enteric, preferably enteric. The term “enteric polymer” refers to a pH-dependent acidic polymer that is insoluble or only slightly soluble at a low pH (e.g. about pH 1 up to but less than pH 3) but becomes soluble at a higher pH (e.g. pH 5 and above). In certain embodiments a pH-dependent polymer may become soluble at a pH range from about pH 5 and above, e.g. from about pH 6 to about pH 9, from about pH 6 to about pH 8, from about pH 5 to about pH 7, or from about pH 5 to about pH 6, which is generally less acidic than the gastric environment and roughly corresponds to pH values in the small intestine. Examples of enteric polymers include but are not limited to methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate, HPMCAS), polyvinyl acetate phthalate (PVAP), methyl methacrylate-methacrylic acid copolymers (Eudragit® L100), shellac, cellulose acetate trimellitate, sodium alginate and zein. The term “non-enteric polymer” refers to a neutral polymer that does not show pH-dependent solubility characteristics. Examples of non-enteric polymers include but are not limited to cellulose derivatives such as Methylcellulose (MC), ethylcellulose (EC), hydroxypropylcellulose (HPC), hydroxyethyl cellulose (HEC), hydroxypropyl methylcellulose (HPMC), poly-vinyl-pyrrolidone (PVP), copovidone such as polyvinylpyrrolidone-vinyl acetate copolymer (PVP-VA), poly (ethylene glycol) PEGs, starch derivatives like cyclodextrin, Soluplus® which is an amphiphilic copolymer consisting of polyethylene glycol, polyvinyl caprolactam, and polyvinyl acetate.

In embodiments, the pharmaceutically acceptable dispersion carrier is a polymer, or more simply the polymer is, selected from the group consisting of hydroxypropyl methylcelluloses and esters thereof, polyvinylpyrrolidones and copolymers thereof, and polymethacrylates and copolymers thereof. The pharmaceutically acceptable dispersion carrier may contain a mixture of two or more polymers.

In an embodiment, the hydroxypropyl methylcelluloses and esters thereof are selected from the group consisting of hydroxypropyl methyl cellulose acetate (HPMCA), hydroxypropyl methyl cellulose (HPMC), hydroxypropyl cellulose (HPC), methyl cellulose, hydroxyethyl methyl cellulose, hydroxyethyl cellulose, hydroxyethyl cellulose acetate, hydroxyethyl ethyl cellulose, hydroxypropyl methyl cellulose acetate succinate (HPMCAS), hydroxypropyl methyl cellulose phthalate (HPMCP), carboxymethyl ethyl cellulose (CMEC), cellulose acetate phthalate (CAP), cellulose acetate succinate (CAS), hydroxypropyl methyl cellulose acetate phthalate (HPMCAP), cellulose acetate trimellitate (CAT), hydroxypropyl methyl cellulose acetate trimellitate (HPMCAT), and carboxymethylcellulose acetate butyrate (CMCAB). In an embodiment, the hydroxypropyl methylcelluloses and esters thereof are selected from the group consisting of hydroxypropyl methylcellulose acetate succinate and hydroxypropyl methylcellulose, in particular hot melt extrusion-grade hydroxypropyl methylcellulose.

In an embodiment, the polyvinylpyrrolidones and copolymers thereof are selected from the group consisting of polyvinylpyrrolidone vinyl acetate copolymer (PVP-VA), polyvinyl alcohols, polyvinyl alcohol polyvinyl acetate copolymers and polyvinylpyrrolidone (PVP). Polyvinylpyrrolidone (PVP) also is commonly denoted polyvidone or povidone. In embodiments, the polyvinylpyrrolidones and copolymers thereof are a polyvinylpyrrolidone vinyl acetate copolymer (PVP-VA).

In an embodiment, the polymethacrylates and copolymers thereof are selected from the group consisting of methacrylic acid-ethyl acrylate copolymer, methacrylic acid-methyl methacrylate copolymer, methyl methacrylate and methacrylic acid copolymer. Polymethacrylates and copolymers thereof are, for example, available under the brand name Eudragit® from Evonik Industries AG. Methacrylic acid-methyl methacrylate copolymer is, for example, available under the brand name Eudragit® L100. In certain embodiments, the polymethacrylates and copolymers thereof are a methylacrylic acid methyl methacrylate copolymer.

In embodiments, the pharmaceutically acceptable dispersion carrier is a polymer, or more simply the polymer is, selected from the group of hydroxypropyl methylcellulose acetate succinate (HPMCAS), polyvinylpyrrolidone vinyl acetate copolymer (PVP-VA), methylacrylic acid methyl methacrylate copolymer (such as Eudragit® L100), and hot melt extrusion-grade hydroxypropyl methylcellulose (HPMC HME).

In certain embodiments, the pharmaceutically acceptable dispersion carrier is hydroxypropyl methylcellulose acetate succinate (HPMCAS). HPMCAS also is known as hypromellose acetate succinate. Hypromellose acetate succinate (HPMCAS) can be obtained by introducing acetyl and succinoyl groups to the hydroxyl groups of the backbone of hydroxypropyl methylcellulose (HPMC) also known as hypromellose. This procedure can be carried out by known methods, for instance by treating IPMC with acetic anhydride and/or with succinic anhydride. Acetic anhydride and succinic anhydride can be reacted with hydroxypropyl methylcellulose (HPMC) under specifically controlled conditions to produce HPMCAS with varying extent of substitution of acetyl and succinoyl groups.

HPMCAS is available in several grades (L, M and H) varying in extent of substitution of acetyl and succinoyl groups, based on the content of acetyl and succinoyl groups (wt %) in the HPMCAS molecule. Any grade of HPMCAS is usable in the solid dispersion of the invention. Preferably, HPMCAS of grade L, M or H is used. In certain embodiments, the pharmaceutically acceptable dispersion carrier is HPMCAS grade L. In certain embodiments, the pharmaceutically acceptable dispersion carrier is HPMCAS grade M. HPMCAS grade M may comprise an acetyl content of 7-11 wt %; a succinoyl content of 10-14 wt %; methoxyl content of 21-25 wt %; and a hydroxypropoxy content of 5-9 wt %. Preferably, HPMCAS grade M (HPMCAS-M) is soluble at pH ≥6. In certain embodiments, the pharmaceutically acceptable dispersion carrier is HPMCAS grade H. Preferably, granular HPMCAS (HPMCAS-G) is used. HPMCAS-G can be used for any grade of HPMCAS, in particular for grade G, such that HPMCAS-MG is used.

In certain embodiments, the pharmaceutically acceptable dispersion carrier is polyvinylpyrrolidone vinyl acetate copolymer (PVP-VA). Polyvinylpyrrolidone vinyl acetate copolymers are linear, random copolymers that are available by free-radical polymerization of the monomers in ratios varying from 70/30 to 30/70 vinyl acetate to vinylpyrrolidone.

In certain embodiments, the pharmaceutically acceptable dispersion carrier is methylacrylic acid methyl methacrylate copolymer, such as Eudragit® L100. As used herein, “methylacrylic acid methyl methacrylate copolymer” is used interchangeably with “methacrylic acid methyl methacrylate copolymer”.

In certain embodiments, the pharmaceutically acceptable dispersion carrier is a hot melt extrusion-grade hydroxypropyl methylcellulose (HPMC HME). HPMC HME refers to a modified grade of hydroxypropyl methylcellulose having low glass transition temperature and melt viscosity, which can be used for making a solid dispersion via hot melt extrusion. HPMC HME is a water soluble amorphous polymer, usually provided as a white to off-white powder, available in three grades, HPMC HME 15 LV, HPMC HME 100 LV and HPMC HME 4M, differing in regard to their molecular weight. Preferably, HPMC HME 15LV having a molecular weight (Mw) below 100 kDa is used. Further preferred, HPMC HME 100LV having a molecular weight (Mw) below 200 kDa is used.

By dispersing compound (1), preferably on a molecular level, in a, for example polymeric, pharmaceutically acceptable dispersion carrier, an amorphous state can be maintained, even when exposed to elevated temperature and/or humidity conditions, and the solid dispersion can reliably provide compound (1) in amorphous form. In embodiments, compound (1) is amorphous. The preferred feature of compound (1) being amorphous can be applied to any embodiment disclosed herein to provide further embodiments according to the invention, in particular, it can be applied to any embodiment of the solid dispersion (including embodiments about the identity of the pharmaceutically acceptable dispersion carrier, the amounts of the components of the solid dispersion, specific dosing regimes, etc), the pharmaceutical composition, the kits, the uses and the processes described herein. The term “amorphous” as used herein refers to a condensed phase where molecules are randomly orientated and characterized by the absence of any microscopic order, with no diffraction peaks by XRPD; an amorphous solid system may be composed of a single chemical entity or may be a multi-component system containing, e.g., an API, polymer and other excipients, without stoichiometric composition. Amorphous solids generally possess crystal-like short range molecular arrangement, but no long range order of molecular packing as found in crystalline solids. The solid state form of a solid may be determined e.g. by x-ray powder diffraction (“XRPD”) or modulated differential scanning calorimetry (“mDSC”).

In embodiments, the solid dispersion comprises, consists essentially of or consists of amorphous compound (1) and a pharmaceutically acceptable dispersion carrier, wherein compound (1) is substantially in amorphous solid state form. In certain embodiments, the substantially amorphous solid state form refers to the solid dispersion comprising at least 80 wt % amorphous compound (1) based on a total weight of 100 wt % of compound (1). In certain embodiments, the substantially amorphous solid state form refers to the solid dispersion comprising at least 85 wt % amorphous compound (1) based on a total weight of 100 wt % of compound (1). In certain embodiments, the substantially amorphous solid state form refers to the solid dispersion comprising at least 90 wt % amorphous compound (1) based on a total weight of 100 wt % of compound (1). In certain embodiments, the substantially amorphous solid state form refers to the solid dispersion comprising at least 95 wt % amorphous compound (1) based on a total weight of 100 wt % of compound (1). In certain embodiments, the substantially amorphous solid-state form refers to the solid dispersion comprising at least 96, 97, 98 or 99 wt % amorphous compound (1) based on a total weight of 100 wt % of compound (1). Thus, the solid dispersion can provide compound (1) in amorphous or essentially amorphous state. Such a solid dispersion can thus be referred to as an amorphous solid dispersion. In embodiments, the solid dispersion thus is an amorphous solid dispersion.

In one embodiment, the solid dispersion comprises a predetermined amount of compound (1) or a pharmaceutically acceptable salt thereof. In this context, a predetermined amount refers to the initial amount of compound (1), or a pharmaceutically acceptable salt thereof used for the preparation of the solid dispersion.

In another embodiment, the solid dispersion comprises a therapeutically effective amount of compound (1) or a pharmaceutically acceptable salt thereof.

In embodiments, compound (1) is present in an amount in a range of from 5 wt % to 95 wt %, based on a total weight of 100 wt % of the solid dispersion. In embodiments, compound (1) is present in an amount in a range of from 25 wt % to 75% wt %, based on a total weight of 100 wt % of the solid dispersion. In embodiments, the pharmaceutically acceptable dispersion carrier is present in an amount in a range of from 5 wt % to 95 wt %, based on a total weight of 100 wt % of the solid dispersion. In embodiments, the pharmaceutically acceptable dispersion carrier is present in an amount in a range of from 25 wt % to 75 wt %, based on a total weight of 100 wt % of the solid dispersion.

In embodiments, compound (1) is present in an amount in a range of from 20 wt % to 50 wt %, based on a total weight of 100 wt % of the solid dispersion. In embodiments, compound (1) is present in an amount in a range of from 25 wt % to 50 wt %, based on a total weight of 100 wt % of the solid dispersion. In embodiments, the pharmaceutically acceptable dispersion carrier is present in an amount in a range of from 50 wt % to 80 wt %, based on a total weight of 100 wt % of the solid dispersion. In embodiments, the pharmaceutically acceptable dispersion carrier is present in an amount in a range of from 50 wt % to 75 wt %, based on a total weight of 100 wt % of the solid dispersion.

The solid dispersion may comprise compound (1) and the pharmaceutically acceptable dispersion carrier in approximately equal weight amounts. In embodiments, the solid dispersion, based on a total weight of 100 wt %, comprises, consists of or consists essentially of approximately 50 wt % of compound (1) and approximately 50 wt % of the pharmaceutically acceptable dispersion carrier.

In embodiments, the solid dispersion, based on a total weight of 100 wt %, comprises, consists of or consists essentially of approximately 25 wt % or 50 wt % of compound (1) and approximately 75 wt % or 50 wt % of the pharmaceutically acceptable dispersion carrier. In embodiments, the weight ratio of compound (1): the pharmaceutically acceptable dispersion carrier in the solid dispersion is of approximately 1:4 to 4:1, preferably 1:3 to 3:1, such as 1:1 to 1:3. In embodiments, the weight ratio of compound (1): the pharmaceutically acceptable dispersion carrier in the solid dispersion is of 1:1 to 1:3 In embodiments, the weight ratio of compound (1): the pharmaceutically acceptable dispersion carrier in the solid dispersion is of approximately 1:1.

As used herein, the terms “approximately” and “about” mean within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range.

In embodiments, the solid dispersion comprises a weight ratio of compound (1):the pharmaceutically acceptable dispersion carrier of approximately 1:4 to 4:1, preferably 1:3 to 3:1, such as 1:1 to 1:3. In embodiments, the solid dispersion comprises a weight ratio of compound (1):the pharmaceutically acceptable dispersion carrier of 1:1 to 1:3 In embodiments, the solid dispersion comprises a weight ratio of compound (1):the pharmaceutically acceptable dispersion carrier of approximately 1:1.

In an embodiment, the solid dispersion is characterized by having an x-ray powder diffractogram (XRPD) comprising no diffraction peak at 2-theta angles equal or below 40.0°, when measured at a temperature in the range of from 20 to 30° C. and with Cu-Kα radiation having a wavelength of 1.54056 Å or 1.54184 Å. “Cu-Kα radiation” as used in the present invention includes Cu-Kal radiation and Cu-Kα1,2 radiation, wherein Cu-Kα1 radiation has a wavelength of 1.54056 Å and Cu-Kα1,2 radiation has an average wavelength of 1.54184 Å.

In another embodiment, the solid dispersion is characterized by having an x-ray powder diffractogram (XRPD) comprising no diffraction peak in the range of from 2.0 to 40.0°, when measured at a temperature in the range of from 20 to 30° C. and with Cu-Kα radiation having a wavelength of 1.54056 Å or 1.54184 Å.

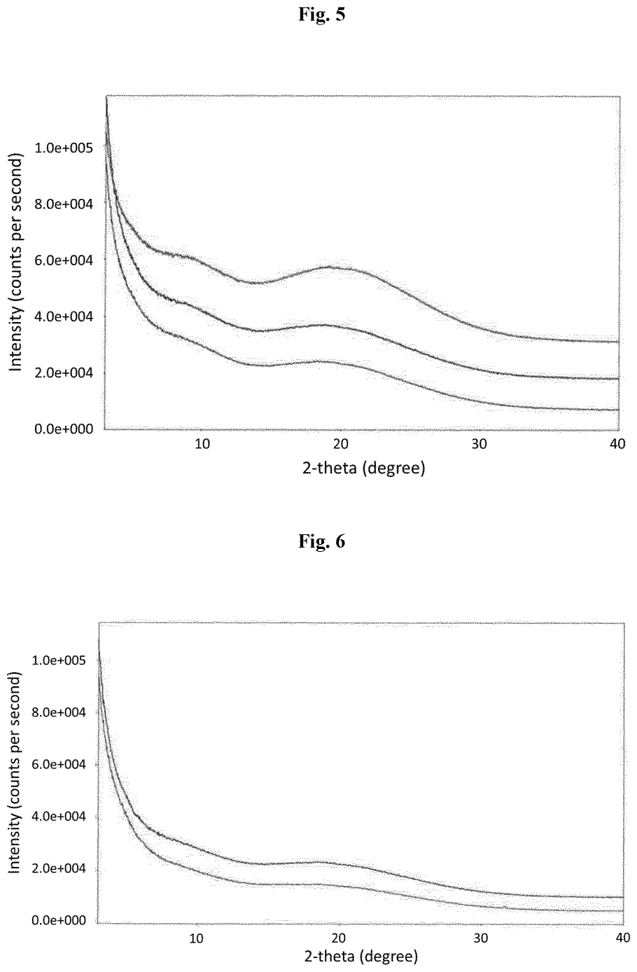

In still another embodiment, the solid dispersion is characterized by having an x-ray powder diffractogram (XRPD) essentially the same as shown in FIG. 5 or FIG. 6 hereinafter, when measured at a temperature in the range of from 20 to 30° C. and with Cu-Kα radiation having a wavelength of 1.54056 Å or 1.54184 Å.

In yet another embodiment, the solid dispersion is characterized by having a differential scanning calorimetry curve comprising a single glass transition temperature (Tg) signal, when measured with modulated differential scanning calorimetry (mDSC) with a modulation amplitude of 1° C./min and a heating rate of 3.0° C./min. Preferably, the single glass transition temperature (Tg) signal is in the range of from 90 to 190° C., preferably of from 110 to 120° C.

In yet other embodiments, the solid dispersion comprises particles characterized by a particle size distribution determined by laser diffraction having
(i) a D90 value of not more than 100 μm, preferably of not more than 90 μm, most preferably of not more than 85 μm; and/or
(ii) a D50 value of not more than 50 μm, preferably of not more than 45 μm, most preferably of not more than 40 μm; and/or
(iii) a D10 value of not more than 20 μm, preferably of not more than 15 μm, most preferably of not more than 13 μm.

In still other embodiments, the solid dispersion comprises particles characterized by a particle size distribution determined by laser diffraction having
(i) a D90 value in the range of from 50 to 100 μm, preferably of from 55 to 90 μm, most preferably of from 60 to 85 μm; and/or
(ii) a D50 value in the range of from 25 to 50 μm, preferably of from 30 to 45 μm, most preferably of from 30 to 40 μm; and/or
(iii) a D10 value in the range of from 1 to 20 μm, preferably of from 5 to 15 μm, most preferably of from 10 to 13 μm.

The term “particle size distribution” as used herein refers to a list of values or a mathematical function that defines the relative amount, typically in mass or volume, of particles present in a sample according to size. Particle size distribution can be characterized by one or more values, such as D90, D50 or D10. The particle size distribution may be determined by means well known to the skilled artisan e.g. by laser diffraction.
“D90”, as used herein, describes the value of particle size at which 90% of the total volume of particles is comprised of particles no larger than the indicated size.
“D50”, as used herein, describes the value of particle size at which 50% of the total volume of particles is comprised of particles no larger than the indicated size.
“D10”, as used herein, describes the value of particle size at which 10% of the total volume of particles is comprised of particles no larger than the indicated size.

A further aspect relates to the use of the solid dispersion as described herein for the preparation of a pharmaceutical composition, wherein the pharmaceutical composition preferably is as defined below.

### Pharmaceutical Composition

Another aspect provides a pharmaceutical composition comprising the solid dispersion as described herein and one or more pharmaceutically acceptable excipients. Another embodiment of the present invention is a pharmaceutical composition comprising a therapeutically effective amount of the solid dispersion as described herein and one or more pharmaceutically acceptable excipients.

Another embodiment of the present invention is a pharmaceutical composition comprising a predetermined amount of the solid dispersion as described herein and one or more pharmaceutically acceptable excipients. In this context, a predetermined amount refers to the initial amount of the solid dispersion used for the preparation of the pharmaceutical composition.

The term “pharmaceutically acceptable excipient” refers to a non-toxic component that does not destroy the pharmacological activity of the compound with which it is formulated. Pharmaceutically acceptable excipients that may be used in the compositions of this invention include fillers, disintegrants, glidants, lubricants, and coating agents. The compositions may comprise further pharmaceutically acceptable excipients selected from buffers, binders, dispersion agents, surfactants, wetting agents, emulsifiers, suspending agents, preservatives, antioxidants, opaquing agents, processing aids, colorants, sweeteners, perfuming agents, flavoring agents, diluents and other known additives usable in the manufacturing of a pharmaceutical product.

The pharmaceutical composition may contain conventional non-toxic pharmaceutically acceptable excipients. In embodiments, the one or more pharmaceutically acceptable excipients are selected from the group consisting of fillers, disintegrants, glidants, lubricants, and coating agents. In embodiments, the pharmaceutical composition comprises a filler, a disintegrant, a glidant and a lubricant. In embodiments, the pharmaceutical composition comprises a filler, a disintegrant, a glidant, a lubricant and a coating agent. It is to be understood that the pharmaceutical composition may comprise one or more excipients of each function, e.g. one or more filler, one or more disintegrants, one or more glidants, one or more lubricants, one or more coating agents.

In embodiments, the filler(s) is(are) selected from the group consisting of microcrystalline cellulose, mannitol and mixtures thereof. In embodiments, the disintegrant(s) is(are) selected from the group consisting of crosslinked sodium carboxymethyl cellulose, also denoted croscarmellose sodium, sodium bicarbonate, crospovidone, sodium starch glycolate and mixtures thereof. In certain embodiments, the disintegrant is croscarmellose sodium. In embodiments, the glidant is colloidal silicon dioxide. In embodiments, the lubricant(s) is(are) selected from the group consisting of stearyl fumarate, magnesium stearate and mixtures thereof. In certain embodiments, the lubricant is sodium stearyl fumarate.

In embodiments, the one or more pharmaceutically acceptable excipients comprise mannitol, microcrystalline cellulose, croscarmellose sodium, colloidal silicon dioxide and sodium stearyl fumarate.

In embodiments, the pharmaceutical composition comprises, consists of or consists essentially of: a solid dispersion comprising compound (1) as defined herein or a pharmaceutically acceptable salt thereof, a pharmaceutically acceptable dispersion carrier, mannitol, microcrystalline cellulose, croscarmellose sodium, colloidal silicon dioxide and sodium stearyl fumarate.

In certain embodiments, the pharmaceutical composition comprises a coating agent, such as when formulated as a film-coated tablet. In embodiments, the coating agent comprises film-forming agents such as polyvinyl alcohol that may be partially hydrolysed, anti-tacking agents such as talc, pigments such as titanium dioxide, glyceryl mono and dicaprylocaprate (GMDCC) and iron oxides such as iron oxide yellow, and lubricants such as sodium lauryl sulphate. Coating agents are commercially available such as under the tradename Opadry® e.g. Opadry® AMB II yellow. In a preferred embodiment, the coating agent does not contain titanium dioxide e.g. is free of titanium dioxide.

In embodiments, the pharmaceutical composition comprises:
(i) a tablet core comprising the solid dispersion as described herein, mannitol, microcrystalline cellulose, croscarmellose sodium, colloidal silicon dioxide and sodium stearyl fumarate; and
(ii) a film coating.

In embodiments, the pharmaceutical composition consists of or consists essentially of:
(i) a tablet core comprising, consisting of or consisting essentially of the solid dispersion as described herein, mannitol, microcrystalline cellulose, croscarmellose sodium, colloidal silicon dioxide and sodium stearyl fumarate; and
(ii) a film coating.

In certain embodiments, the film coating is a non-functional film coat. In one embodiment, the film-coating does not contain titanium dioxide.

In embodiments, the pharmaceutical composition, based on a total weight of 100 wt % of the pharmaceutical composition, comprises the solid dispersion as described herein in a range of from 25 wt % to 65 wt %, preferably of from 35 wt % to 60 wt % or of from 25 wt % to 35 wt % or of from 27 wt % to 31 wt %, still preferably of approximately 30 wt %.

In embodiments, the pharmaceutical composition, based on a total weight of 100 wt % of the pharmaceutical composition, comprises:
in a range of from 25 wt % to 65 wt % of the solid dispersion as described herein; and/or
in a range of from 25 wt % to 65 wt % of one or more fillers; and/or
in a range of from 4 wt % to 10 wt % of disintegrant; and/or
in a range of from 1 wt % to 2 wt % of glidant; and/or
in a range of from 1 wt % to 2 wt % of lubricant; and/or
optionally a range of from 2 wt % to 5 wt % of coating agent.

In the present and any of the following embodiments referring to wt % of components of the pharmaceutical composition, it is to be understood that the sum of the ranges or amounts of all components does not exceed 100 wt %.

In further embodiments, the pharmaceutical composition, based on a total weight of 100 wt % of the pharmaceutical composition, comprises:
in a range of from 30 wt % to 60 wt %, preferably 35 wt % to 60 wt %, of the solid dispersion as described herein; and/or
in a range of from 30 wt % to 60 wt %, preferably 35 wt % to 60 wt %, of one or more fillers; and/or
in a range of from 4 wt % to 7 wt % of disintegrant; and/or
in a range of from 1 wt % to 1.5 wt % of glidant; and/or
in a range of from 1 wt % to 1.5 wt % of lubricant; and/or
optionally a range of from 3 wt % to 5 wt % of coating agent.

In still further embodiments, the pharmaceutical composition, based on a total weight of 100 wt % of the pharmaceutical composition, comprises:
in a range of from 25 wt % to 35 wt % of the solid dispersion as described herein; and/or
in a range of from 55 wt % to 65 wt % of one or more fillers; and/or
in a range of from 4 wt % to 7 wt % of disintegrant; and/or
in a range of from 1 wt % to 2 wt % of glidant; and/or
in a range of from 1 wt % to 2 wt % of lubricant; and/or
optionally a range of from 3 wt % to 5 wt % of coating agent.

In an embodiment, the pharmaceutical composition, based on a total weight of 100 wt % of the pharmaceutical composition, comprises:
in a range of from 28 wt % to 30 wt % of the solid dispersion as described herein; and/or
in a range of from 57 wt % to 62 wt % of one or more fillers; and/or
in a range of from 4 wt % to 5 wt % of disintegrant; and/or
in a range of from 1.4 wt % to 1.5 wt % of glidant; and/or
in a range of from 1.4 wt % to 1.5 wt % of lubricant; and/or
optionally approximately 4 wt % of coating agent.

In this embodiment, preferably, the lower limit of the range of solid dispersion, fillers, disintegrant, glidant and lubricant refers to the pharmaceutical composition with coating agent, while the higher limit of the same range refers to the pharmaceutical composition without coating agent.

In embodiments, the pharmaceutical composition, based on a total weight of 100 wt % of the pharmaceutical composition, consists essentially of or consists of:
in a range of from 25 wt % to 65 wt % of the solid dispersion as described herein;
in a range of from 25 wt % to 65 wt % of one or more fillers;
in a range of from 4 wt % to 10 wt % of disintegrant;
in a range of from 1 wt % to 2 wt % of glidant; and
in a range of from 1 wt % to 2 wt % of lubricant, wherein the sum of the ranges all components does not exceed 100 wt %.

In further embodiments, the pharmaceutical composition, based on a total weight of 100 wt % of the pharmaceutical composition, consists essentially of or consists of:
in a range of from 30 wt % to 60 wt %, preferably 35 wt % to 60 wt %, of the solid dispersion as described herein;
in a range of from 30 wt % to 60 wt %, preferably 35 wt % to 60 wt %, of one or more fillers;
in a range of from 4 wt % to 7 wt % of disintegrant;
in a range of from 1 wt % to 1.5 wt % of glidant; and
in a range of from 1 wt % to 1.5 wt % of lubricant, wherein the sum of the ranges all components does not exceed 100 wt %.

In still further embodiments, the pharmaceutical composition, based on a total weight of 100 wt % of the pharmaceutical composition, consists essentially of or consists of:
in a range of from 25 wt % to 35 wt % of the solid dispersion as described herein;
in a range of from 55 wt % to 65 wt % of one or more fillers;
in a range of from 4 wt % to 7 wt % of disintegrant;
in a range of from 1 wt % to 2 wt % of glidant; and
in a range of from 1 wt % to 2 wt % of lubricant, wherein the sum of the ranges all components does not exceed 100 wt %.

In embodiments, the pharmaceutical composition comprises compound (1) in a range of from 10 to 20 wt % based on a total weight of 100 wt % of the pharmaceutical composition. In embodiments, the pharmaceutical composition comprises compound (1) in an amount of approximately 15 wt % based on a total weight of 100 wt % of the pharmaceutical composition.

In a particular embodiment, the pharmaceutical composition comprises, consists essentially of or consists of: approximately 15 wt % compound (1), approximately 15 wt % hypromellose acetate succinate, approximately 36 wt % microcrystalline cellulose, approximately 24 wt % mannitol, approximately 7 wt % croscarmellose sodium, approximately 1.5 wt % colloidal silicone dioxide and approximately 1.5 wt % sodium stearyl fumarate, based on a total weight of 100 wt % of the pharmaceutical composition.

In a particular embodiment, the pharmaceutical composition comprises, consists essentially of or consists of: approximately 15 wt % compound (1), approximately 15 wt % hypromellose acetate succinate, approximately 20 wt % microcrystalline cellulose, approximately 42 wt % mannitol, approximately 5 wt % croscarmellose sodium, approximately 1.5 wt % colloidal silicone dioxide and approximately 1.5 wt % sodium stearyl fumarate, based on a total weight of 100 wt % of the pharmaceutical composition.

In a particular embodiment, the pharmaceutical composition comprises, consists essentially of or consists of: approximately 14 wt % compound (1), approximately 43 wt % hypromellose acetate succinate, approximately 19 wt % microcrystalline cellulose, approximately 24 wt % mannitol, approximately 7 wt % croscarmellose sodium, approximately 1.5 wt % colloidal silicone dioxide and approximately 1.5 wt % sodium stearyl fumarate, based on a total weight of 100 wt % of the pharmaceutical composition.

In a particular embodiment, the pharmaceutical composition comprises, consists essentially of or consists of: approximately 17.5 wt % compound (1), approximately 17.5 wt % hypromellose acetate succinate, approximately 30 wt % microcrystalline cellulose, approximately 25 wt % mannitol, approximately 7 wt % croscarmellose sodium, approximately 1.5 wt % colloidal silicone dioxide and approximately 1.5 wt % sodium stearyl fumarate, based on a total weight of 100 wt % of the pharmaceutical composition.

In a particular embodiment, the pharmaceutical composition comprises, consists essentially of or consists of: approximately 15 wt % compound (1), approximately 47 wt % hypromellose acetate succinate, approximately 15 wt % microcrystalline cellulose, approximately 15 wt % mannitol, approximately 5 wt % croscarmellose sodium, approximately 1 wt % colloidal silicone dioxide and approximately 1 wt % sodium stearyl fumarate, based on a total weight of 100 wt % of the pharmaceutical composition.

In one embodiment, the pharmaceutical composition comprises, consists essentially of or consists of: approximately 15 mg compound (1), approximately 15 mg hypromellose acetate succinate, approximately 36 mg microcrystalline cellulose, approximately 24 mg mannitol, approximately 7 mg croscarmellose sodium, approximately 1.5 mg colloidal silicone dioxide and approximately 1.5 mg sodium stearyl fumarate.

In one embodiment, the pharmaceutical composition comprises, consists essentially of or consists of: approximately 15 mg compound (1), approximately 15 mg hypromellose acetate succinate, approximately 20 mg microcrystalline cellulose, approximately 42 mg mannitol, approximately 5 mg croscarmellose sodium, approximately 1.5 mg colloidal silicone dioxide and approximately 1.5 mg sodium stearyl fumarate.

In one embodiment, the pharmaceutical composition comprises, consists essentially of or consists of: approximately 60 mg compound (1), approximately 60 mg hypromellose acetate succinate, approximately 80 mg microcrystalline cellulose, approximately 168 mg mannitol, approximately 20 mg croscarmellose sodium, approximately 6 mg colloidal silicone dioxide and approximately 6 mg sodium stearyl fumarate.

In an embodiment, the pharmaceutical composition is characterized by having an x-ray powder diffractogram (XRPD) comprising no diffraction peak at 2-theta angles equal or below 10.0°, when measured at a temperature in the range of from 20 to 30° C. and with Cu-Kα radiation having a wavelength of 1.54056 Å or 1.54184 Å.

In another embodiment, the pharmaceutical composition is characterized by having an x-ray powder diffractogram (XRPD) comprising no diffraction peak at 2-theta angles equal or below 9.0°, when measured at a temperature in the range of from 20 to 30° C. and with Cu-Kα radiation having a wavelength of 1.54056 Å or 1.54184 Å.

In yet another embodiment, the pharmaceutical composition is characterized by having an x-ray powder diffractogram (XRPD) comprising no diffraction peak at 2-theta angles equal or below 6.5°, when measured at a temperature in the range of from 20 to 30° C. and with Cu-Kα radiation having a wavelength of 1.54056 Å or 1.54184 Å.

In another embodiment, the pharmaceutical composition is characterized by having an x-ray powder diffractogram (XRPD) comprising no diffraction peak in the range of from 2.0 to 10.0°, when measured at a temperature in the range of from 20 to 30° C. and with Cu-Kα radiation having a wavelength of 1.54056 Å or 1.54184 Å.

In still another embodiment, the pharmaceutical composition is characterized by having an x-ray powder diffractogram (XRPD) comprising no diffraction peak in the range of from 2.0 to 9.0°, when measured at a temperature in the range of from 20 to 30° C. and with Cu-Kα radiation having a wavelength of 1.54056 Å or 1.54184 Å.

In one embodiment, the pharmaceutical composition is characterized by having an x-ray powder diffractogram (XRPD) comprising no diffraction peak in the range of from 2.0 to 6.5°, when measured at a temperature in the range of from 20 to 30° C. and with Cu-Kα radiation having a wavelength of 1.54056 Å or 1.54184 Å In another embodiment, the pharmaceutical composition is characterized by having an x-ray powder diffractogram (XRPD) comprising no diffraction peak in the range of from 2.0 to 10.0°, when measured at a temperature in the range of from 20 to 30° C. and with Cu-Kα radiation having a wavelength of 1.54056 Å or 1.54184 Å.

In yet another embodiment, the pharmaceutical composition is characterized by having an x-ray powder diffractogram (XRPD) comprising no diffraction peak at a 2-Theta angle of (5.9 0.2°), when measured at a temperature in the range of from 20 to 30° C. and with Cu-Kα radiation having a wavelength of 1.54056 Å or 1.54184 Å.

In a further embodiment, the pharmaceutical composition is characterized by having an x-ray powder diffractogram (XRPD) comprising no diffraction peak at a 2-Theta angle of (6.2±0.2°), when measured at a temperature in the range of from 20 to 30° C. and with Cu-Kα radiation having a wavelength of 1.54056 Å or 1.54184 Å.

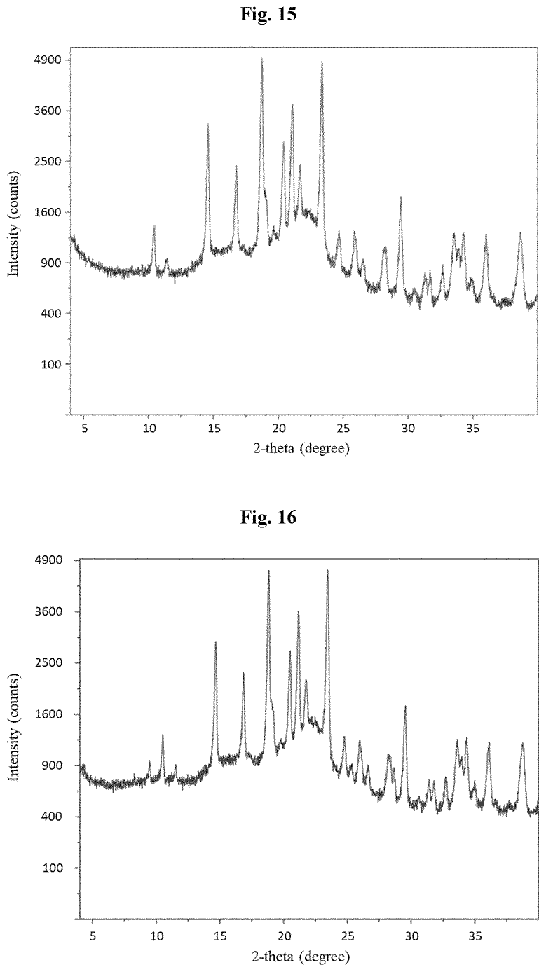

In another embodiment, the pharmaceutical composition is characterized by having an x-ray powder diffractogram (XRPD) essentially the same as shown in FIG. 15 or FIG. 16 hereinafter, when measured at a temperature in the range of from 20 to 30° C. and with Cu-Kα radiation having a wavelength of 1.54056 Å or 1.54184 Å.

To be used for treatment, the solid dispersion or the pharmaceutical composition may be included or formulated into appropriate dosage units to facilitate administration. The solid dispersion or the pharmaceutical composition thus may be formulated in suitable dosage unit formulations appropriate for each route of administration. Typical pharmaceutical unit formulations include for example tablets, pills, capsules, suppositories, lozenges, troches, solutions particularly solutions for infusion, elixirs, syrups, sachets, emulsions, or dispersible powders. Dosage forms and formulations of active ingredients are known in the art and dosage units may generally be prepared in any conventional manner.

The solid dispersion or the pharmaceutical composition preferably may be administered by oral routes of administration and may be formulated in suitable dosage unit formulations. The pharmaceutical composition may be administered as a tablet, hard or soft gelatin capsule, pill, granules or a suspension. In embodiments, the pharmaceutical composition is in the form of a tablet, of granules or of a capsule. In preferred embodiments, the pharmaceutical composition is in the form of a film-coated tablet. Suitable tablets may be obtained, for example, by mixing the solid dispersion with known excipients, for example inert diluents, carriers, disintegrants, adjuvants, surfactants, binders and/or lubricants. Tablets may be compressed from the solid dispersion or a mixture of the solid dispersion with excipients or from pellets thereof. In other embodiments, the solid dispersion, a mixture of the solid dispersion with excipients or pellets thereof may be packed into capsules.

Although oral administration may be preferred in view of compliance, routes of administration are not limited to oral administration, but the solid dispersion or the pharmaceutical composition may be administered parenterally, e.g. intramuscular, intraperitoneal, intravenous, transdermal or subcutaneous injection or by implant, or enterically, nasal, vaginal, rectal, or topical administration.

The solid dispersion or the pharmaceutical composition may be administered at therapeutically effective amounts or be included in a dosage form in a therapeutically effective amount. A therapeutically effective amount refers to an amount effective at dosages and for periods of time necessary to achieve a desired therapeutic result and is the minimum amount necessary to prevent, ameliorate, or treat a disease or disorder, or which any toxic or detrimental effects of the compound is outweighed by the therapeutically beneficial effects. As used herein, the terms “active ingredient”, “active pharmaceutical ingredient”, “active substance” and “API” refer to a component that is intended to furnish pharmacological activity or other direct effect, such as compound (1).

The pharmaceutical composition preferably contains a therapeutically effective amount of compound (1). In embodiments, a therapeutically effective amount of compound (1) may be portioned in one or more individual dosage unit formulations, and thus several individual dosage unit formulations may contain a portion of a therapeutically effective amount of compound (1). In embodiments, a tablet, a portion of granules or a capsule may contain between 5 mg and 100 mg of compound (1). In embodiments, a tablet, a portion of granules or a capsule may contain between 15 mg and 80 mg of compound (1). In embodiments, a tablet, a portion of granules or a capsule may contain between 15 mg and 30 mg of compound (1). In embodiments, a tablet, a portion of granules or a capsule may contain approximately 15, 30 or 60 mg of compound (1).

For storage, the solid dispersion or the pharmaceutical composition may be packaged in appropriate containments (i.e. a means to contain the solid dispersion or pharmaceutical composition). Such containments may be selected from bags, blisters, bottles, ampoules, and vials. The containments may be made of suitable packaging materials.

Typical packaging materials are selected from glass, polyethylene, polypropylene, polystyrene, polymethyl methacrylate, low-density polyethylene (LDPE), polyvinyl chloride (PVC), polyvinylidene chloride (PVDC), polychlorotrifluoroethylene (PCTFE), cyclic olefin copolymers (COC), cyclic olefin polymers (COP), polyethylene terephthalate (PET), glycol-modified polyethylene terephthalate (PETg), aluminum, polyamide, and any combinations thereof. In a particular embodiment, the solid dispersion is packaged into double low-density polyethylene (LDPE) bags. In still another embodiment, the pharmaceutical composition is packaged into high-density polyethylene (HDPE) bottles. Preferably, the HDPE bottles further contain a desiccant. Typical desiccants may be selected from activated alumina, aerogel, benzophenone (as anion), bentonite clay, calcium chloride, calcium oxide, calcium sulfate, cobalt(II) chloride, copper(II) sulfate, lithium chloride, lithium bromide, magnesium chloride hexahydrate, magnesium sulfate, magnesium perchlorate, molecular sieve, phosphorous pentoxide, potassium carbonate, potassium hydroxide, rice, silica gel, sodium chlorate, sodium chloride, sodium hydroxide, sodium sulfate, sucrose and sulfuric acid. In a preferred embodiment the desiccant is silica gel.

A further embodiment of the present invention refers to a kit comprising:
the solid dispersion or pharmaceutical composition as described herein; and
a means to contain said solid dispersion or pharmaceutical composition, preferably a high-density polyethylene bottle; and
optionally a desiccant, preferably silica gel.
   Use for Treatment and/or Prevention of Oncological and/or Hyperproliferative Diseases

In an embodiment is provided compound (1) in the dose regime as described herein for use in the treatment and/or prevention of cancer. In addition, a further aspect relates to compound (1) and the dose regime described herein for use in the treatment and/or prevention of cancer, wherein the patient already received in the past one or more anti-cancer treatments or therapies. A further embodiment provides compound (1) for use in the treatment and/or prevention of cancer, wherein the patient already received a different first, second or further line treatment. A different treatment comprises administration of an anti-cancer therapy, drug or agent different from and not comprising compound (1). The previously performed first, second or further line treatment is not correlated with administration of compound (1) and the previously performed first, second or further line treatment is finished or has ended before a treatment comprising the administration of compound (1) is performed.

A further aspect relates to a method of treating and/or preventing cancer, wherein the method comprises the step of administering compound (1) in the dose regime described herein to a patient. In an embodiment, such method comprises administering to a human in need of such treatment the disclosed therapeutically effective amount of compound (1). A further aspect relates to a method of treating and/or preventing cancer, wherein the method comprises the step of administering compound (1) in the dose regime described herein to a patient, wherein the patient already received a different first, second or further line treatment. In an embodiment, such method comprises administering to a human in need of such treatment a therapeutically effective amount of compound (1) as described herein, wherein the patient already received a different first, second or further line treatment.

An embodiment relates to the use of compound (1) in the dose regime described herein in the manufacture of a medicament for the treatment and/or prevention of cancer.

The solid dispersion and pharmaceutical compositions as described herein can be used as medicaments in the dose regime as described herein. Particularly, the solid dispersion and pharmaceutical compositions as described herein can be used for the treatment and/or prevention of oncological and/or hyperproliferative disorders, in particular in anti-cancer therapy, in the dose regime as described herein.

According to an aspect is provided the solid dispersion, as described herein, for use as a medicament, in the dose regime as described herein. According to another aspect is provided the pharmaceutical composition, as described herein, for use as a medicament, in the dose regime as described herein. Another embodiment of the present invention is the solid dispersion or the pharmaceutical composition for treating or preventing a disease, in the dose regime as described herein.

According to an aspect is provided the solid dispersion, as described herein, for use as an anti-cancer medicament, in the dose regime as described herein. According to another aspect is provided the pharmaceutical composition, as described herein, for use as an anti-cancer medicament, in the dose regime as described herein.

In an embodiment is provided the solid dispersion as described herein for use in the treatment and/or prevention of a disease or a disorder modulated by HER2, particularly an oncological and/or hyperproliferative disease, in the dose regime as described herein. A further embodiment provides the pharmaceutical composition as described herein for use in the treatment and/or prevention of a disease or disorder modulated by HER2, particularly an oncological and/or hyperproliferative disease, in the dose regime as described herein. Another aspect refers to the solid dispersion described herein or the pharmaceutical composition described herein for use in a method of treating and/or preventing a disease or disorder modulated by HER2, particularly an oncological or hyperproliferative disease, in the dose regime as described herein.

A further aspect relates to a method of treating and/or preventing a disease or disorder modulated by HER2, particularly an oncological and/or hyperproliferative disease, wherein the method comprises the step of administering the solid dispersion described herein or the pharmaceutical composition described herein in the dose regime as described herein to a patient. In an embodiment, such method comprises administering to a human in need of such treatment a therapeutically effective amount of the solid dispersion or pharmaceutical composition described herein.

A related aspect relates to the use of the solid dispersion described herein or the pharmaceutical composition described herein in the dose regime as described herein in the manufacture of a medicament. An embodiment relates to the use of the solid dispersion described herein or the pharmaceutical composition described herein in the dose regime as described herein in the manufacture of a medicament for the treatment and/or prevention of a disease or disorder modulated by HER2, particularly an oncological and/or hyperproliferative disease.

In one aspect, is provided the solid dispersion or the pharmaceutical composition as described herein for use in the treatment and/or prevention of a disease and/or condition in the dose regime as described herein, wherein the inhibition of wild type and/or mutant HER2 is of therapeutic benefit, particularly for the treatment and/or prevention of a disease and/or condition in the dose regime as described herein, wherein the inhibition of HER2 exon 20 mutant protein is of therapeutic benefit. Examples of such diseases and/or conditions include, but are not limited to, oncological and/or hyperproliferative diseases such as cancer.

One aspect relates to the solid dispersion described herein for use in the treatment and/or prevention of an oncological and/or hyperproliferative disease in the dose regime as described herein. A further aspect relates to the pharmaceutical composition as described herein for use in the treatment and/or prevention of an oncological and/or hyperproliferative disease in the dose regime as described herein.

As used herein, the term “hyperproliferative disease” refers to conditions wherein cell growth is increased over normal levels. Hyperproliferative diseases include malignant diseases, such as cancers, and non-malignant diseases. In preferred embodiments, the hyperproliferative disorder is cancer. As used herein, the term “oncological disease” refers to a disease or medical condition associated with cancer or cancer indication. Cancers can be classified by the type of tissue in which the cancer originates (histological type) and by primary site, or the location in the body, where the cancer first developed.

In an embodiment, the oncological and/or hyperproliferative disease is cancer.

In an embodiment is provided the solid dispersion as described herein for use in the treatment and/or prevention of cancer in the dose regime as described herein. A further embodiment provides the pharmaceutical composition as described herein for use in the treatment and/or prevention of cancer in the dose regime as described herein. Another aspect refers to the solid dispersion described herein or the pharmaceutical composition described herein for use in a method of treating and/or preventing cancer in the dose regime as described herein.

In addition, a further aspect relates to the solid dispersion or pharmaceutical composition as described herein in the dose regime described herein for use in the treatment and/or prevention of cancer, wherein the patient already received in the past one or more anti-cancer treatments or therapies. A further embodiment provides the solid dispersion or pharmaceutical composition as described herein in the dose regime described herein for use in the treatment and/or prevention of cancer, wherein the patient already received a different first, second or further line treatment.

A further aspect relates to a method of treating and/or preventing cancer, wherein the method comprises the step of administering the solid dispersion described herein or the pharmaceutical composition described herein in the dose regime as described herein to a patient. In an embodiment, such method comprises administering to a human in need of such treatment the disclosed therapeutically effective amount of compound (1).

A further aspect relates to a method of treating and/or preventing cancer, wherein the method comprises the step of administering the solid dispersion described herein or the pharmaceutical composition described herein in the dose regime described herein to a patient, wherein the patient already received a different first, second or further line treatment. In an embodiment, such method comprises administering to a human in need of such treatment a therapeutically effective amount of compound (1) as described herein, wherein the patient already received a different first, second or further line treatment.

An embodiment relates to the use of the solid dispersion described herein or the pharmaceutical composition described herein in the dose regime as described herein in the manufacture of a medicament for the treatment and/or prevention of cancer.

In embodiments, the cancer is HER2 overexpressed, HER2 amplified and/or HER2 mutant. In embodiments, the cancer is HER2 exon 20 mutant cancer. In embodiments, the oncological and/or hyperproliferative disease is a HER2 overexpressed, HER2 amplified and/or HER2 mutant cancer.

“HER2 overexpressed” as used herein refers to a cancer, where the cells of the cancer or tumor express HER2 at levels detectable by immunohistochemistry (e.g. IHC 2+ and IHC 3+) and/or methods assaying ERBB2 messenger RNA.

“HER2 amplified” as used herein refers to a cancer where the cancer or tumor cells exhibit more than 2, in particular more than 3, 4, 5, 6, 7, 8, 9 or 10, preferably more than 6, copies of the HER2 gene ERBB2.

HER2 expression, gene copy number and amplification can be measured, for example, by determining nucleic acid sequencing (e.g., sequencing of genomic DNA or cDNA), measuring mRNA expression, measuring protein abundance, or a combination thereof. HER2 testing methods include immunohistochemistry (IHC), fluorescence in situ hybridization (FISH), chromogenic in situ hybridization (CISH), ELISAs, and RNA quantification using techniques such as RT-PCR, microarray analysis and Next Generation Sequencing (NGS). HER2 expression in or on the cancer sample cells can be compared to a reference cell. The reference cell can be a non-cancer cell obtained from the same subject as the sample cell. The reference cell can be a non-cancer cell obtained from a different subject or a population of subjects.

When the cancer is HER2 overexpressed and/or HER2 amplified in or on a cell, the cancer can be referred to as being “HER2 positive”.

“HER2 mutant” as used herein refers to a cancer harbouring at least one mutation, i.e. an alteration in the nucleic acid sequence of the HER2 gene and/or an alteration in the amino acid sequence of the HER2 protein, including but not limited to those listed below. Mutations can be found with any method known to the skilled person, such as molecular diagnostic methods including but not limited to Polymerase Chain Reaction (PCR), Single Strand Conformational Polymorphism (SSCP), Denaturing Gradient Gel Electrophoresis (DGGE), Heteroduplex analysis, Restriction fragment length polymorphism (RFLP), Next Generation Sequencing (NGS) and Whole Exome Sequencing.

“Cancer with HER2 exon 20 mutation” or “HER2 exon 20 mutant cancer” as used herein refers to a cancer where the cancer or tumor cells harbour at least one HER2 exon 20 mutation including but not limited to the mutations listed below.

ERBB2 (HER2) exon 20 encodes for a part of the kinase domain and ranges from amino acids 769 to 835. Every mutation, insertion, duplication or deletion within this region is defined as an exon 20 mutation including the following mutations: p.A772_G773insMMAY; p.Y772_A775_dup (YVMA); p.A775_G776insYVMA; p.Y772insYVMA; p.M774delinsWLV; p.A775_G776insSVMA; p.A775_G776insVVMA; p.A775_G776insYVMS; p.A775_G776insC; p.A776_delinsVC; p.A776_delinsLC; p.A776_delinsVV; p.A776_delinsAVGC; p.A776_delinsIC; p.A776_V777delinsCVC; p.V777_insE; p.G778_P780dup (GSP); p.G776_delinsVC (“p.” is referring to the HER2 protein).

In addition oncogenic HER2 mutations exist outside of exon 20 including the following mutations: p.S310F; p.R678Q; p.L755S; p.L755A; p.L755P; p.S310Y; p.S310A; p.V842I; p.D769Y; p.D769H; p.R103Q; p.G1056S; p.I767M; p.L869R; p.L869R; p.T733I; p.T862A; p.V697L; p.V777L; p.V777M; p.R929W; p.D277H; p.D277Y; p.G660D (“p.” is referring to the HER2 protein).

In embodiments, the oncological and/or hyperproliferative disease or the cancer is one of the following cancers, tumors or other proliferative diseases, without being restricted thereto: Cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis); appendix;
cancers/tumors/carcinomas of the testis: e.g. seminomas, non-seminomas;
Gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), HER2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; nonfluorineunctional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer; neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
peripheral nervous system cancer;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (BchlorineL), chronic T-cell lymphocytic leukemia (TchlorineL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP).

All cancers/tumors/carcinomas mentioned above which are characterized by their specific location/origin in the body are meant to include both the primary tumors and the metastatic tumors derived therefrom. Preferably, the cancer as defined herein (including in any embodiment referring to e.g. cancer types) is metastatic, advanced, and/or unresectable.

All cancers/tumors/carcinomas mentioned above may be further differentiated by their histopathological classification:
Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;
Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas.

In some embodiments, the cancer is a solid tumor. In some embodiments, the cancer is manifested by at least one solid tumor.

In some embodiments, the cancer is selected from the group consisting of brain cancer, breast cancer, endocrine cancer, gastrointestinal cancer, gynecologic cancer, head and neck tumor, lung cancer, nervous system cancer, and skin cancer.

Preferably, said brain cancer is a glioblastoma or a glioma.

Preferably, said breast cancer is lobular breast cancer. In addition or in alternative, said breast cancer is preferably metastatic.

Preferably, said endocrine cancer is nerve sheath tumor, more preferably HER2 mutant nerve sheath tumor.

Preferably, said gastrointestinal cancer is selected from the group consisting of anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer and small bowel cancer.

In addition or in alternative, said gastrointestinal cancer may be a gastrointestinal neuroendocrine tumor, preferably HER2 mutant. Still preferably, said gastrointestinal cancer is selected from the group consisting of gastric adenocarcinoma, gastroesophageal junction adenocarcinoma and esophageal adenocarcinoma, in particular metastatic gastric adenocarcinoma, metastatic gastroesophageal junction adenocarcinoma and metastatic esophageal adenocarcinoma.

Preferably, said gynecologic cancer is selected from the group consisting of cervical cancer, uterine cancer, endometrial cancer and ovarian cancer.

Preferably, said head and neck tumor is a salivary gland cancer or tumor.

Preferably, said lung cancer is non-small cell lung cancer (NSCLC).

Preferably, said nervous system cancer is peripheral nervous system cancer, more preferably HER2 amplified peripheral nervous system cancer.

Preferably, said skin cancer is not a melanoma, i.e. non-melanoma skin cancer.

In some embodiments, the cancer is selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer. In some embodiments, the cancer is HER2 overexpressed, HER2 amplified and/or HER2 mutant (in particular HER2 exon 20 mutant) cancer selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer.

In some embodiments, the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In some embodiments, the cancer is HER2 overexpressed, HER2 amplified and/or HER2 mutant (in particular HER2 exon 20 mutant) cancer selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In some embodiments, the cancer is selected from the group consisting of brain cancer, breast cancer, biliary tract cancer, bladder cancer, cervical cancer, uterine cancer, colorectal cancer, endometrial cancer, skin cancer, gastric cancer, esophagus tumor, head and neck tumor, gastrointestinal cancer, gallbladder tumor, kidney cancer, liver cancer, lung cancer and prostate cancer.

In embodiments, the cancer is HER2 overexpressed, HER2 amplified and/or HER2 mutant (in particular HER2 exon 20 mutant) cancer selected from brain cancer, breast cancer, biliary tract cancer, bladder cancer, cervical cancer, uterine cancer, colorectal cancer, endometrial cancer, skin cancer, gastric cancer, esophagus tumor, head and neck tumor, gastrointestinal cancer, gallbladder tumor, kidney cancer, liver cancer, lung cancer and prostate cancer.

In other embodiments, the cancer is selected from the group consisting of breast cancer, bladder cancer, colorectal cancer, gastrointestinal cancer, esophageal cancer or lung cancer. In further embodiments, the cancer is selected from cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer). In still further embodiments, the cancer is NSCLC. In still further embodiments, the cancer is HER2 exon 20 mutant NSCLC. In further embodiments, the cancer is unresectable. In still further embodiments, the cancer is unresectable HER2 exon 20 mutant NSCLC.

In a preferred embodiment said cancer is advanced or metastatic. In a further preferred embodiment, said cancer is advanced and metastatic. In a further preferred embodiment, when said cancer is metastatic, the metastases are located in the lung, lymph node or bone. In a further preferred embodiment, the cancer is advanced cancer including metastases and the metastases are located in the lung, lymph node or bone.

In addition or in alternative, said cancer can be unresectable.

In a preferred embodiment the cancer is unresectable advanced cancer comprising solid tumors and solid metastases and the metastases are located in the lung- or lymph node-tissue or bone. In a preferred embodiment the cancer is advanced NSCLC comprising solid, unresectable tumors and metastases and the metastases are located in the lung- or lymph node-tissue or bone.

In a preferred embodiment the cancer is HER2 exon 20 mutant advanced NSCLC comprising solid, unresectable tumors and metastases and the metastases are located in the lung- or lymph node-tissue or bone.

In an embodiment, the cancer is advanced, unresectable or metastatic NSCLC harbouring a HER2 mutation, wherein said HER2 mutation is in the tyrosine kinase domain. Preferably, in this embodiment, the solid dispersion or the pharmaceutical composition as described herein is administered as first line of therapy. Still preferably, in this embodiment, the solid dispersion or the pharmaceutical composition as described herein is administered as second or further line of therapy.

In embodiments, the cancer is HER2 positive metastatic breast cancer. Preferably, in this embodiment, the solid dispersion or the pharmaceutical composition as described herein is administered as first line of therapy. Still preferably, in this embodiment, the solid dispersion or the pharmaceutical composition as described herein is administered as second or further line of therapy.

In embodiments, the cancer is HER2 positive metastatic gastric adenocarcinoma, metastatic gastroesophageal junction adenocarcinoma or metastatic esophageal adenocarcinoma. Preferably, in this embodiment, the solid dispersion or the pharmaceutical composition as described herein is administered as first line of therapy. Still preferably, in this embodiment, the solid dispersion or the pharmaceutical composition as described herein is administered as second or further line of therapy.

A further aspect relates to compound (1), the solid dispersion or the pharmaceutical composition for use as a medicament in the dose regime as described herein, particularly, for the treatment and/or prevention of oncological and/or hyperproliferative disorders, such as cancer, wherein compound (1), the solid dispersion or the pharmaceutical composition is administered:
to a fasted subject, and/or
in combination with a medicament that increases gastric pH, preferably a proton-pump inhibitor (PPI), an antacid or an antihistamine, and/or
to a subject having a gastric pH in the range of from about 1 to 7, preferably to a subject having a gastric pH in the range of from about 1 to 5.

In embodiments, the solid dispersion as described herein or the pharmaceutical composition as described herein is administered in the dose regime as described herein:
to a fasted subject, and/or
in combination with a medicament that increases gastric pH, preferably a proton-pump inhibitor (PPI), an antacid or an antihistamine.

Another aspect relates to compound (1) as defined above for use in the treatment and/or prevention of an oncological and/or hyperproliferative disease, wherein compound (1) is administered in the dose regime as described herein:
to a fasted subject, and/or
in combination with a medicament that increases gastric pH.

In embodiments, compound (1), the solid dispersion or the pharmaceutical composition is administered in the dose regime as described herein to a fasted subject.

The term “subject” as used herein refers to a human, e.g. a human suffering from, at risk of suffering from, or potentially capable of suffering from cancer.

In embodiments, compound (1), the solid dispersion or the pharmaceutical composition is administered in the dose regime as described herein in combination with a medicament that increases gastric pH, preferably a proton-pump inhibitor (PPI), an antacid or an antihistamine.

In embodiments, compound (1), the solid dispersion or the pharmaceutical composition is administered in the dose regime as described herein to a fasted subject and in combination with a medicament that increases gastric pH, preferably a proton-pump inhibitor (PPI), an antacid or an antihistamine.

In embodiments, compound (1), the solid dispersion or the pharmaceutical composition is administered in the dose regime as described herein to a subject having a gastric pH in the range of from about 1 to 7.

In embodiments, compound (1), the solid dispersion or the pharmaceutical composition is administered in the dose regime as described herein to a subject having a gastric pH in the range of from about 1 to 5.

A “fasted subject” as used herein refers to one who has not eaten for at least eight hours, preferably for at least ten hours, typically overnight, prior to administration of the solid dispersion or the pharmaceutical composition or a dosage form thereof. A fasted subject conveniently may receive compound (1), the solid dispersion, the pharmaceutical composition or a dosage form thereof with water after at least eight or 10 hours fasting. Thereafter, no food may be taken for a period of, e.g. 4 hours although small quantities of water may be taken after, e.g. 2 hours after receiving medicament.

In embodiments, a fasted subject is one who has not eaten for at least two hours prior to the administration of the solid dispersion or the pharmaceutical composition described herein and/or who does not eat for at least one hour after administration of the solid dispersion or the pharmaceutical composition described herein.

In embodiments, a fasted subject is one who has not eaten for approximately two hours prior to the administration of the solid dispersion or the pharmaceutical composition described herein and who does not eat for approximately one hour after administration of the solid dispersion or the pharmaceutical composition described herein. In these embodiments, the fasted subject may be referred to as “modified fasted subject”.

“Medicament that increase gastric pH” refers to a class of medications that neutralize stomach acidity. Medicaments that neutralize gastric acid may reduce pepsin activity. In embodiments, the medicament that increases gastric pH is a proton-pump inhibitor. The term “proton-pump inhibitor” (PPI) refers to a class of medications that cause a profound and prolonged reduction of stomach acid production. In embodiments, they are suppressors of gastric acid secretion. In embodiments, PPIs which may be administered in combination with compound (1), the solid dispersion or the pharmaceutical composition, include, without being restricted thereto, rabeprazole, omeprazole, pantoprazole, esomeprazole, lansoprazole, dexlansoprazole, and ilaprazole. Rabeprazole is a proton-pump inhibitor indicated for diseases that profit with an increased gastric pH such as reflux esophagitis.

In embodiments, the medicament that increases gastric pH is an antacid. The term “antacid” refers to a class of medications that neutralize stomach acidity. In embodiments, antacids which may be administered in combination with compound (1), the solid dispersion or the pharmaceutical composition, include, without being restricted thereto, salts of aluminium, calcium, magnesium or sodium, such as aluminium hydroxide, magnesium hydroxide, magnesium oxide, magnesium carbonate, calcium carbonate and sodium bicarbonate.

In embodiments, the medicament that increases gastric pH is an antihistamine, in particular a H₂ receptor antagonist. The term “H₂ receptor antagonist” refers to a class of medications that block the action of histamine in the stomach. In embodiments, antihistamines which may be administered in combination with compound (1), the solid dispersion or the pharmaceutical composition, include, without being restricted thereto, cimetidine, ranitidine, famotidine, nizatidine, roxatidine, lafutidine, lavoltidine and niperotidine.

Compound (1), the solid dispersion, the pharmaceutical composition or a dosage form thereof and the medicament that increases gastric pH, can be administered simultaneously, concurrently, sequentially, or successively. The term “simultaneous” refers to the administration of both compounds/compositions at substantially the same time. The term “concurrent” refers to administration of the active ingredients within the same general time period, for example on the same day(s) but not necessarily at the same time. The term “sequential” administration includes administration of one active ingredient during a first time period, for example over the course of a few hours, days or a week, using one or more doses, followed by administration of the other active ingredient during a second time period, for example over the course of a few hours, days or a week, using one or more doses. An overlapping schedule may also be employed, which includes administration of the active ingredients on different days over the treatment period, not necessarily according to a regular sequence. The term “successive” administration, alternatively, refers to an administration where the second administration step is carried out immediately once the administration of the first compounds has been finished. Variations of these general administration forms may also be employed.

In embodiments, compound (1), the solid dispersion, the pharmaceutical composition or a dosage form thereof is administered in the dose regime as described herein after a medicament that increases gastric pH, preferably a proton-pump inhibitor (PPI), an antacid or an antihistamine.

In another aspect, the present invention relates to the solid dispersion or the pharmaceutical composition as described herein for use in the treatment and/or prevention of an oncological and/or hyperproliferative disease as defined herein, wherein the solid dispersion or the pharmaceutical composition is administered in the dose regime as described herein in combination with a cytostatic and/or cytotoxic active substance and/or in combination with radiotherapy and/or immunotherapy.

In another aspect, the present invention relates to a combination of the solid dispersion or the pharmaceutical composition as described herein in the dose regime as described herein with a cytostatic and/or cytotoxic active substance and/or in combination with radiotherapy and/or immunotherapy for use in the treatment and/or prevention of cancer.

The solid dispersion or the pharmaceutical composition as described herein may be used on their own or in combination with one or more other pharmacologically active substances such as state-of-the-art or standard-of-care compounds, such as e.g. cell proliferation inhibitors, anti-angiogenic substances, steroids or immune modulators/checkpoint inhibitors, and the like. Pharmacologically active substances which may be administered in combination with the solid dispersion or the pharmaceutical composition as described herein, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors), inhibitors are for example (anti-)growth factor antibodies, (anti-)growth factor receptor antibodies and tyrosine kinase inhibitors, such as for example cetuximab, gefitinib, afatinib, nintedanib, imatinib, lapatinib, bosutinib, bevacizumab, pertuzumab and trastuzumab); antimetabolites (e.g. antifolates such as methotrexate, raltitrexed, pyrimidine analogues such as 5fluorouracil (5fluorineU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumor antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors, ERK inhibitors, KRAS inhibitors (e.g. KRAS G12C inhibitors), signalling pathway inhibitors (e.g. SOS1 inhibitors), FLT3 inhibitors, BRD4 inhibitors, IGF-1R inhibitors, TRATLR2 agonists, Bcl-xL inhibitors, Bcl-2 inhibitors, Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors, immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), T-cell engagers (e.g. bi-specific T-cell engagers (BiTEs®) like e.g. CD3 x BCMA, CD3 x CD33, CD3 x CD19), PSMA x CD3), tumor vaccines and various chemotherapeutic agents such as amifostin, anagrelid, clodronat, filgrastin, interferon, interferon alpha, leucovorin, procarbazine, levamisole, mesna, mitotane, pamidronate and porfimer.

Doses and dose regimes of compound (1) It is a purpose of the present invention to provide a safe and efficacious dose regime for the administration of compound (1) or a pharmaceutical composition comprising compound (1) in the described dose regime. This dose regime is especially helpful for use in the treatment of cancer. In addition, this dose regime of compound (1) or a pharmaceutical composition comprising compound (1) is especially helpful in a method of treating a patient suffering from cancer. In addition, the dose regime seems also to be suitable as a second or further line treatment, wherein the patient already received in the past one or more kinds of cancer treatments.

As used herein “daily dose” or “total daily dose” refers to the amount of active substance, i.e. compound (1), which is administered to the patient within a 24 h timeframe. The 24 h timeframe does not necessarily start at noon or midnight.

It was surprisingly discovered that the use of compound (1) in the treatment of cancer in a daily dose of at least 30 mg is safe and efficacious as described above and in the example below.

In preferred embodiments, compound (1) is administered in a daily dose of at least 60 mg. Alternatively, the method of treating a patient suffering from cancer described above comprises administering compound (1) in a daily dose of at least 60 mg.

In preferred embodiments, compound (1) is administered in a daily dose of at least 80 mg. Alternatively, a method of treating a patient suffering from cancer comprising administering compound (1) in a daily dose of at least 80 mg.

In preferred embodiments, compound (1) is administered in a daily dose of at least 120 mg. Alternatively, the method of treating a patient suffering from cancer comprises administering compound (1) in a daily dose of at least 120 mg.

In preferred embodiments, compound (1) is administered in a daily dose of at least 180 mg. Alternatively, the method of treating a patient suffering from cancer comprises administering compound (1) in a daily dose of at least 180 mg.

In preferred embodiments, compound (1) is administered in a daily dose of at least 200 mg. Alternatively, the method of treating a patient suffering from cancer comprising administering compound (1) in a daily dose of at least 200 mg.

In preferred embodiments, compound (1) is administered in a daily dose of at least 240 mg. Alternatively, the method of treating a patient suffering from cancer comprises administering compound (1) in a daily dose of at least 240 mg.

In preferred embodiments, compound (1) is administered in a daily dose of at least 300 mg. Alternatively, the method of treating a patient suffering from cancer comprises administering compound (1) in a daily dose of at least 300 mg.

In preferred embodiments, compound (1) is administered in a daily dose of 30 mg to 600 mg. Alternatively, the method of treating a patient suffering from cancer comprises administering compound (1) in a daily dose of 30 mg to 600 mg.

In preferred embodiments, compound (1) is administered in a daily dose of 60 mg to 600 mg. Alternatively, the method of treating a patient suffering from cancer comprises administering compound (1) in a daily dose of 60 mg to 600 mg.

In preferred embodiments, compound (1) is administered in a daily dose of 80 mg to 600 mg. Alternatively, the method of treating a patient suffering from cancer comprises administering compound (1) in a daily dose of 80 mg to 600 mg.

In preferred embodiments, compound (1) is administered in a daily dose of 120 mg to 600 mg. Alternatively, the method of treating a patient suffering from cancer comprises administering compound (1) in a daily dose of 120 mg to 600 mg.

In a further preferred embodiment, compound (1) is administered in a daily dose of 30 mg to 300 mg. Alternatively, the method of treating a patient suffering from cancer comprises administering compound (1) in a daily dose of 30 mg to 300 mg.

In a further preferred embodiment, compound (1) is administered in a daily dose of 60 mg to 300 mg. Alternatively, the method of treating a patient suffering from cancer comprises administering compound (1) in a daily dose of 60 mg to 300 mg.

In a further preferred embodiment, compound (1) is administered in a daily dose of 80 mg to 300 mg. Alternatively, the method of treating a patient suffering from cancer comprises administering compound (1) in a daily dose of 80 mg to 300 mg.

In a further preferred embodiment, compound (1) is administered in a daily dose of 120 mg to 300 mg. Alternatively, the method of treating a patient suffering from cancer comprises administering compound (1) in a daily dose of 120 mg to 300 mg.

In preferred embodiments, compound (1) is administered in a daily dose of 30 mg, 60 mg, 120 mg, 180 mg, 200 mg, 240 mg, 300 mg, 360 mg, 400 mg, 420 mg, 480 mg, 500 mg, 540 mg or 600 mg. Alternatively, the method of treating a patient suffering from cancer comprises administering compound (1) in a daily dose of 30 mg, 60 mg, 120 mg, 180 mg, 200 mg, 240 mg, 300 mg, 360 mg, 400 mg, 420 mg, 480 mg, 500 mg, 540 mg or 600 mg.

In preferred embodiments, compound (1) is administered in a daily dose of 30 mg, 60 mg, 120 mg, 180 mg, 200 mg, 240 mg, 300 mg, 360 mg, 420 mg, 480 mg, 540 mg or 600 mg. Alternatively, the method of treating a patient suffering from cancer comprises administering compound (1) in a daily dose of 30 mg, 60 mg, 120 mg, 180 mg, 200 mg, 240 mg, 300 mg, 360 mg, 420 mg, 480 mg, 540 mg or 600 mg.

In preferred embodiments, compound (1) is administered in a daily dose of 30 mg, 60 mg, 120 mg, 180 mg, 200 mg, 240 mg or 300 mg. Alternatively, the method of treating a patient suffering from cancer comprises administering compound (1) in a daily dose of 30 mg, 60 mg, 120 mg, 180 mg, 200 mg, 240 mg or 300 mg.

In preferred embodiments, compound (1) is administered in a daily dose of 30 mg. Alternatively, the method of treating a patient suffering from cancer comprises administering compound (1) in a daily dose of 30 mg.

In preferred embodiments, compound (1) is administered in a daily dose of 60 mg. Alternatively, the method of treating a patient suffering from cancer comprises administering compound (1) in a daily dose of 60 mg.

In preferred embodiments, compound (1) is administered in a daily dose of 120 mg. Alternatively, the method of treating a patient suffering from cancer comprises administering compound (1) in a daily dose of 120 mg.

In preferred embodiments, compound (1) is administered in a daily dose of 180 mg. Alternatively, the method of treating a patient suffering from cancer comprises administering compound (1) in a daily dose of 180 mg.

In preferred embodiments, compound (1) is administered in a daily dose of 200 mg. Alternatively, the method of treating a patient suffering from cancer comprises administering compound (1) in a daily dose of 200 mg.

In preferred embodiments, compound (1) is administered in a daily dose of 240 mg. Alternatively, the method of treating a patient suffering from cancer comprises administering compound (1) in a daily dose of 240 mg.

In preferred embodiments, compound (1) is administered in a daily dose of 300 mg. Alternatively, the method of treating a patient suffering from cancer comprises administering compound (1) in a daily dose of 300 mg.

In preferred embodiments, compound (1) is administered in a daily dose of 360 mg. Alternatively, the method of treating a patient suffering from cancer comprises administering compound (1) in a daily dose of 360 mg.

In preferred embodiments, compound (1) is administered in a daily dose of 400 mg. Alternatively, the method of treating a patient suffering from cancer comprises administering compound (1) in a daily dose of 400 mg.

In preferred embodiments, compound (1) is administered in a daily dose of 420 mg. Alternatively, the method of treating a patient suffering from cancer comprises administering compound (1) in a daily dose of 420 mg.

In preferred embodiments, compound (1) is administered in a daily dose of 480 mg. Alternatively, the method of treating a patient suffering from cancer comprises administering compound (1) in a daily dose of 480 mg.

In preferred embodiments, compound (1) is administered in a daily dose of 500 mg. Alternatively, the method of treating a patient suffering from cancer comprises administering compound (1) in a daily dose of 500 mg.

In preferred embodiments, compound (1) is administered in a daily dose of 540 mg. Alternatively, the method of treating a patient suffering from cancer comprises administering compound (1) in a daily dose of 540 mg.

In preferred embodiments, compound (1) is administered in a daily dose of 600 mg. Alternatively, the method of treating a patient suffering from cancer comprises administering compound (1) in a daily dose of 600 mg.

Within a preferred embodiment, compound (1) is administered once or twice daily. This means, that the daily dose is either administered as a single dose or the daily dose is divided in two separate administrations, each administered at a different time point at that day, i.e. within 24 h.

In a preferred embodiment, compound (1) is administered once daily. In a preferred embodiment, compound (1) is administered as a single dose within 24 h.

In a preferred embodiment, compound (1) is administered twice daily. In a preferred embodiment, compound (1) is administered twice within 24 h.

In preferred embodiments, each of the two daily administrations of compound (1) corresponds to half the daily dose. An easy and error-proof application scheme can be provided by administering the required daily dose of compound (1) in two doses comprising the same amount.

In a further preferred embodiment, compound (1) is administered at least for 21 consecutive days. In further preferred embodiments, compound (1) is administered for 21 days multiplied by X, wherein X is a natural number equal or larger than 1. It is also possible, that in the overall cancer treatment between the treatment times including administering compound (1) a dose-free time-interval is included.

In some embodiments, compound (1) is administered once daily in a daily dose of 60 mg, 120 mg, 180 mg, 240 mg, 300 mg, 360 mg, 400 mg, 420 mg, 480 mg, 500 mg, 540 mg or 600 mg or compound (1) is administered twice daily in a daily dose of 30 mg, 60 mg, 120 mg, 200 mg, 300 mg, 360 mg, 400 mg, 420 mg, 480 mg, 500 mg, 540 mg or 600 mg. Alternatively, the method of treating a patient suffering from cancer comprises administering compound (1) once daily in a daily dose of 60 mg, 120 mg, 180 mg, 240 mg, 300 mg, 360 mg, 400 mg, 420 mg, 480 mg, 500 mg, 540 mg or 600 mg or compound (1) is administered twice daily in a daily dose of 30 mg, 60 mg, 120 mg, 200 mg, 300 mg, 360 mg, 400 mg, 420 mg, 480 mg, 500 mg, 540 mg or 600 mg.

In some embodiments, compound (1) is administered once daily in a daily dose of 60 mg, 120 mg, 180 mg, 240 mg, 300 mg, 360 mg, 420 mg, 480 mg, 540 mg or 600 mg or compound (1) is administered twice daily in a daily dose of 30 mg, 60 mg, 120 mg, 200 mg, 300 mg, 360 mg, 420 mg, 480 mg, 540 mg or 600 mg. Alternatively, the method of treating a patient suffering from cancer comprises administering compound (1) once daily in a daily dose of 60 mg, 120 mg, 180 mg, 240 mg, 300 mg, 360 mg, 420 mg, 480 mg, 540 mg or 600 mg or compound (1) is administered twice daily in a daily dose of 30 mg, 60 mg, 120 mg, 200 mg, 300 mg, 360 mg, 420 mg, 480 mg, 540 mg or 600 mg.

In some embodiments, compound (1) is administered once daily in a daily dose of 60 mg, 120 mg, 180 mg, 240 mg or 300 mg or compound (1) is administered twice daily in a daily dose of 30 mg, 60 mg, 120 mg, 200 mg or 300 mg. Alternatively, the method of treating a patient suffering from cancer comprises administering compound (1) once daily in a daily dose of 60 mg, 120 mg, 180 mg, 240 mg or 300 mg or compound (1) twice daily in a daily dose of 30 mg, 60 mg, 120 mg, 200 mg or 300 mg.

In some embodiments, compound (1) is administered once daily in a daily dose of 60 mg, 120 mg, 180 mg, 240 mg, 300 mg, 360 mg, 400 mg, 420 mg, 480 mg, 500 mg, 540 mg or 600 mg. Alternatively, the method of treating a patient suffering from cancer comprises administering compound (1) once daily in a daily dose of 60 mg, 120 mg, 180 mg, 240 mg, 300 mg, 360 mg, 400 mg, 420 mg, 480 mg, 500 mg, 540 mg or 600 mg.

In some embodiments, compound (1) is administered once daily in a daily dose of 60 mg, 120 mg, 180 mg, 240 mg, 300 mg, 360 mg, 420 mg, 480 mg, 540 mg or 600 mg. Alternatively, the method of treating a patient suffering from cancer comprises administering compound (1) once daily in a daily dose of 60 mg, 120 mg, 180 mg, 240 mg, 300 mg, 360 mg, 420 mg, 480 mg, 540 mg or 600 mg.

In some embodiments, compound (1) is administered once daily in a daily dose of 60 mg, 120 mg, 180 mg, 240 mg or 300 mg. Alternatively, the method of treating a patient suffering from cancer comprises administering compound (1) once daily in a daily dose of 60 mg, 120 mg, 180 mg, 240 mg or 300 mg.

In preferred embodiments, compound (1) is administered once daily in a daily dose of 60 mg. Alternatively, the method of treating a patient suffering from cancer comprises administering compound (1) once daily in a daily dose of 60 mg.

In preferred embodiments, compound (1) is administered once daily in a daily dose of 120 mg. Alternatively, the method of treating a patient suffering from cancer comprises administering compound (1) once daily in a daily dose of 120 mg.

In preferred embodiments, compound (1) is administered once daily in a daily dose of 180 mg. Alternatively, the method of treating a patient suffering from cancer comprises administering compound (1) once daily in a daily dose of 180 mg.

In preferred embodiments, compound (1) is administered once daily in a daily dose of 240 mg. Alternatively, the method of treating a patient suffering from cancer comprises administering compound (1) once daily in a daily dose of 240 mg.

In preferred embodiments, compound (1) is administered once daily in a daily dose of 300 mg. Alternatively, the method of treating a patient suffering from cancer comprises administering compound (1) once daily in a daily dose of 300 mg.

In preferred embodiments, compound (1) is administered once daily in a daily dose of 360 mg. Alternatively, the method of treating a patient suffering from cancer comprises administering compound (1) once daily in a daily dose of 360 mg.

In preferred embodiments, compound (1) is administered once daily in a daily dose of 400 mg. Alternatively, the method of treating a patient suffering from cancer comprises administering compound (1) once daily in a daily dose of 400 mg.

In preferred embodiments, compound (1) is administered once daily in a daily dose of 420 mg. Alternatively, the method of treating a patient suffering from cancer comprises administering compound (1) once daily in a daily dose of 420 mg.

In preferred embodiments, compound (1) is administered once daily in a daily dose of 480 mg. Alternatively, the method of treating a patient suffering from cancer comprises administering compound (1) once daily in a daily dose of 480 mg.

In preferred embodiments, compound (1) is administered once daily in a daily dose of 500 mg. Alternatively, the method of treating a patient suffering from cancer comprises administering compound (1) once daily in a daily dose of 500 mg.

In preferred embodiments, compound (1) is administered once daily in a daily dose of 540 mg. Alternatively, the method of treating a patient suffering from cancer comprises administering compound (1) once daily in a daily dose of 540 mg.

In preferred embodiments, compound (1) is administered once daily in a daily dose of 600 mg. Alternatively, the method of treating a patient suffering from cancer comprises administering compound (1) once daily in a daily dose of 600 mg.

In some embodiments, compound (1) is administered twice daily in a daily dose of 30 mg, 60 mg, 120 mg, 200 mg, 300 mg, 360 mg, 400 mg, 420 mg, 480 mg, 500 mg, 540 mg or 600 mg. Alternatively, the method of treating a patient suffering from cancer comprises administering compound (1) twice daily in a daily dose of 30 mg, 60 mg, 120 mg, 200 mg, 300 mg, 360 mg, 400 mg, 420 mg, 480 mg, 500 mg, 540 mg or 600 mg.

In some embodiments, compound (1) is administered twice daily in a daily dose of 30 mg, 60 mg, 120 mg, 200 mg, 300 mg, 360 mg, 420 mg, 480 mg, 540 mg or 600 mg. Alternatively, the method of treating a patient suffering from cancer comprises administering compound (1) twice daily in a daily dose of 30 mg, 60 mg, 120 mg, 200 mg, 300 mg, 360 mg, 420 mg, 480 mg, 540 mg or 600 mg.

In some embodiments, compound (1) is administered twice daily in a daily dose of 30 mg, 60 mg, 120 mg, 200 mg or 300 mg. Alternatively, the method of treating a patient suffering from cancer comprises administering compound (1) twice daily in a daily dose of 30 mg, 60 mg, 120 mg, 200 mg or 300 mg.

In preferred embodiments, compound (1) is administered twice daily in a daily dose of 30 mg. Alternatively, the method of treating a patient suffering from cancer comprises administering compound (1) twice daily in a daily dose of 30 mg. Preferably, in these embodiments, each of the two daily administrations is of 15 mg.

In preferred embodiments, compound (1) is administered twice daily in a daily dose of 60 mg. Alternatively, the method of treating a patient suffering from cancer comprises administering compound (1) twice daily in a daily dose of 60 mg. Preferably, in these embodiments, each of the two daily administrations is of 30 mg.

In preferred embodiments, compound (1) is administered twice daily in a daily dose of 120 mg. Alternatively, the method of treating a patient suffering from cancer comprises administering compound (1) twice daily in a daily dose of 120 mg. Preferably, in these embodiments, each of the two daily administrations is of 60 mg.

In preferred embodiments, compound (1) is administered twice daily in a daily dose of 200 mg. Alternatively, the method of treating a patient suffering from cancer comprises administering compound (1) twice daily in a daily dose of 200 mg. Preferably, in these embodiments, each of the two daily administrations is of 100 mg.

In preferred embodiments, compound (1) is administered twice daily in a daily dose of 300 mg. Alternatively, the method of treating a patient suffering from cancer comprises administering compound (1) twice daily in a daily dose of 300 mg. Preferably, in these embodiments, each of the two daily administrations is of 150 mg.

In preferred embodiments, compound (1) is administered twice daily in a daily dose of 360 mg. Alternatively, the method of treating a patient suffering from cancer comprises administering compound (1) twice daily in a daily dose of 360 mg. Preferably, in these embodiments, each of the two daily administrations is of 180 mg.

In preferred embodiments, compound (1) is administered twice daily in a daily dose of 400 mg. Alternatively, the method of treating a patient suffering from cancer comprises administering compound (1) twice daily in a daily dose of 400 mg. Preferably, in these embodiments, each of the two daily administrations is of 200 mg.

In preferred embodiments, compound (1) is administered twice daily in a daily dose of 420 mg. Alternatively, the method of treating a patient suffering from cancer comprises administering compound (1) twice daily in a daily dose of 420 mg. Preferably, in these embodiments, each of the two daily administrations is of 210 mg.

In preferred embodiments, compound (1) is administered twice daily in a daily dose of 480 mg. Alternatively, the method of treating a patient suffering from cancer comprises administering compound (1) twice daily in a daily dose of 480 mg. Preferably, in these embodiments, each of the two daily administrations is of 240 mg.

In preferred embodiments, compound (1) is administered twice daily in a daily dose of 500 mg. Alternatively, the method of treating a patient suffering from cancer comprises administering compound (1) twice daily in a daily dose of 500 mg. Preferably, in these embodiments, each of the two daily administrations is of 250 mg.

In preferred embodiments, compound (1) is administered twice daily in a daily dose of 540 mg. Alternatively, the method of treating a patient suffering from cancer comprises administering compound (1) twice daily in a daily dose of 540 mg. Preferably, in these embodiments, each of the two daily administrations is of 270 mg.

In preferred embodiments, compound (1) is administered twice daily in a daily dose of 600 mg. Alternatively, the method of treating a patient suffering from cancer comprises administering compound (1) twice daily in a daily dose of 600 mg. Preferably, in these embodiments, each of the two daily administrations is of 300 mg.

In some embodiments, compound (1) is administered orally, preferably as a tablet, once daily in a daily dose of 60 mg, 120 mg, 180 mg, 240 mg, 300 mg, 360 mg, 400 mg, 420 mg, 480 mg, 500 mg, 540 mg or 600 mg. Alternatively, the method of treating a patient suffering from cancer comprises administering compound (1) orally, preferably as a tablet, once daily in a daily dose of 60 mg, 120 mg, 180 mg, 240 mg, 300 mg, 360 mg, 400 mg, 420 mg, 480 mg, 500 mg, 540 mg or 600 mg.

In some embodiments, compound (1) is administered orally, preferably as a tablet, once daily in a daily dose of 60 mg, 120 mg, 180 mg, 240 mg, 300 mg, 360 mg, 420 mg, 480 mg, 540 mg or 600 mg. Alternatively, the method of treating a patient suffering from cancer comprises administering compound (1) orally, preferably as a tablet, once daily in a daily dose of 60 mg, 120 mg, 180 mg, 240 mg, 300 mg, 360 mg, 420 mg, 480 mg, 540 mg or 600 mg.

In some embodiments, compound (1) is administered orally, preferably as a tablet, once daily in a daily dose of 60 mg, 120 mg, 180 mg, 240 mg or 300 mg. Alternatively, the method of treating a patient suffering from cancer comprises administering compound (1) orally, preferably as a tablet, once daily in a daily dose of 60 mg, 120 mg, 180 mg, 240 mg or 300 mg.

In some embodiments, compound (1) is administered orally, preferably as a tablet, once daily in a daily dose of 60 mg. Alternatively, the method of treating a patient suffering from cancer comprises administering compound (1) orally, preferably as a tablet, once daily in a daily dose of 60 mg.

In some embodiments, compound (1) is administered orally, preferably as a tablet, once daily in a daily dose of 120 mg. Alternatively, the method of treating a patient suffering from cancer comprises administering compound (1) orally, preferably as a tablet, once daily in a daily dose of 120 mg.

In some embodiments, compound (1) is administered orally, preferably as a tablet, once daily in a daily dose of 180 mg. Alternatively, the method of treating a patient suffering from cancer comprises administering compound (1) orally, preferably as a tablet, once daily in a daily dose of 180 mg.

In some embodiments, compound (1) is administered orally, preferably as a tablet, once daily in a daily dose of 240 mg. Alternatively, the method of treating a patient suffering from cancer comprises administering compound (1) orally, preferably as a tablet, once daily in a daily dose of 240 mg.

In some embodiments, compound (1) is administered orally, preferably as a tablet, once daily in a daily dose of 300 mg. Alternatively, the method of treating a patient suffering from cancer comprises administering compound (1) orally, preferably as a tablet, once daily in a daily dose of 300 mg.

In some embodiments, compound (1) is administered orally, preferably as a tablet, once daily in a daily dose of 360 mg. Alternatively, the method of treating a patient suffering from cancer comprises administering compound (1) orally, preferably as a tablet, once daily in a daily dose of 360 mg.

In some embodiments, compound (1) is administered orally, preferably as a tablet, once daily in a daily dose of 400 mg. Alternatively, the method of treating a patient suffering from cancer comprises administering compound (1) orally, preferably as a tablet, once daily in a daily dose of 400 mg.

In some embodiments, compound (1) is administered orally, preferably as a tablet, once daily in a daily dose of 420 mg. Alternatively, the method of treating a patient suffering from cancer comprises administering compound (1) orally, preferably as a tablet, once daily in a daily dose of 420 mg.

In some embodiments, compound (1) is administered orally, preferably as a tablet, once daily in a daily dose of 480 mg. Alternatively, the method of treating a patient suffering from cancer comprises administering compound (1) orally, preferably as a tablet, once daily in a daily dose of 480 mg.

In some embodiments, compound (1) is administered orally, preferably as a tablet, once daily in a daily dose of 500 mg. Alternatively, the method of treating a patient suffering from cancer comprises administering compound (1) orally, preferably as a tablet, once daily in a daily dose of 500 mg.

In some embodiments, compound (1) is administered orally, preferably as a tablet, once daily in a daily dose of 540 mg. Alternatively, the method of treating a patient suffering from cancer comprises administering compound (1) orally, preferably as a tablet, once daily in a daily dose of 540 mg.

In some embodiments, compound (1) is administered orally, preferably as a tablet, once daily in a daily dose of 600 mg. Alternatively, the method of treating a patient suffering from cancer comprises administering compound (1) orally, preferably as a tablet, once daily in a daily dose of 600 mg.

In some embodiments, compound (1) is administered orally, preferably as a tablet, twice daily in a daily dose of 30 mg, 60 mg, 120 mg, 200 mg, 300 mg, 360 mg, 400 mg, 420 mg, 480 mg, 500 mg, 540 mg or 600 mg. Alternatively, the method of treating a patient suffering from cancer comprises administering compound (1) orally, preferably as a tablet, twice daily in a daily dose of 30 mg, 60 mg, 120 mg, 200 mg, 300 mg, 360 mg, 400 mg, 420 mg, 480 mg, 500 mg, 540 mg or 600 mg.

In some embodiments, compound (1) is administered orally, preferably as a tablet, twice daily in a daily dose of 30 mg, 60 mg, 120 mg, 200 mg, 300 mg, 360 mg, 420 mg, 480 mg, 540 mg or 600 mg. Alternatively, the method of treating a patient suffering from cancer comprises administering compound (1) orally, preferably as a tablet, twice daily in a daily dose of 30 mg, 60 mg, 120 mg, 200 mg, 300 mg, 360 mg, 420 mg, 480 mg, 540 mg or 600 mg.

In some embodiments, compound (1) is administered orally, preferably as a tablet, twice daily in a daily dose of 30 mg, 60 mg, 120 mg, 200 mg or 300 mg. Alternatively, the method of treating a patient suffering from cancer comprises administering compound (1) orally, preferably as a tablet, twice daily in a daily dose of 30 mg, 60 mg, 120 mg, 200 mg or 300 mg. In some embodiments, compound (1) is administered orally, preferably as a tablet, twice daily in a daily dose of 30 mg. Alternatively, the method of treating a patient suffering from cancer comprises administering compound (1) orally, preferably as a tablet, twice daily in a daily dose of 30 mg. Preferably, in these embodiments, each of the two daily administrations is of 15 mg. In some embodiments, compound (1) is administered orally, preferably as a tablet, twice daily in a daily dose of 60 mg. Alternatively, the method of treating a patient suffering from cancer comprises administering compound (1) orally, preferably as a tablet, twice daily in a daily dose of 60 mg. Preferably, in these embodiments, each of the two daily administrations is of 30 mg. In some embodiments, compound (1) is administered orally, preferably as a tablet, twice daily in a daily dose of 120 mg. Alternatively, the method of treating a patient suffering from cancer comprises administering compound (1) orally, preferably as a tablet, twice daily in a daily dose of 120 mg. Preferably, in these embodiments, each of the two daily administrations is of 60 mg. In some embodiments, compound (1) is administered orally, preferably as a tablet, twice daily in a daily dose of 200 mg. Alternatively, the method of treating a patient suffering from cancer comprises administering compound (1) orally, preferably as a tablet, twice daily in a daily dose of 200 mg. Preferably, in these embodiments, each of the two daily administrations is of 100 mg.

In some embodiments, compound (1) is administered orally, preferably as a tablet, twice daily in a daily dose of 300 mg. Alternatively, the method of treating a patient suffering from cancer comprises administering compound (1) orally, preferably as a tablet, twice daily in a daily dose of 300 mg. Preferably, in these embodiments, each of the two daily administrations is of 150 mg.

In some embodiments, compound (1) is administered orally, preferably as a tablet, twice daily in a daily dose of 360 mg. Alternatively, the method of treating a patient suffering from cancer comprises administering compound (1) orally, preferably as a tablet, twice daily in a daily dose of 360 mg. Preferably, in these embodiments, each of the two daily administrations is of 180 mg.

In some embodiments, compound (1) is administered orally, preferably as a tablet, twice daily in a daily dose of 400 mg. Alternatively, the method of treating a patient suffering from cancer comprises administering compound (1) orally, preferably as a tablet, twice daily in a daily dose of 400 mg. Preferably, in these embodiments, each of the two daily administrations is of 200 mg.

In some embodiments, compound (1) is administered orally, preferably as a tablet, twice daily in a daily dose of 420 mg. Alternatively, the method of treating a patient suffering from cancer comprises administering compound (1) orally, preferably as a tablet, twice daily in a daily dose of 420 mg. Preferably, in these embodiments, each of the two daily administrations is of 210 mg.

In some embodiments, compound (1) is administered orally, preferably as a tablet, twice daily in a daily dose of 480 mg. Alternatively, the method of treating a patient suffering from cancer comprises administering compound (1) orally, preferably as a tablet, twice daily in a daily dose of 480 mg. Preferably, in these embodiments, each of the two daily administrations is of 240 mg.

In some embodiments, compound (1) is administered orally, preferably as a tablet, twice daily in a daily dose of 500 mg. Alternatively, the method of treating a patient suffering from cancer comprises administering compound (1) orally, preferably as a tablet, twice daily in a daily dose of 500 mg. Preferably, in these embodiments, each of the two daily administrations is of 250 mg.

In some embodiments, compound (1) is administered orally, preferably as a tablet, twice daily in a daily dose of 540 mg. Alternatively, the method of treating a patient suffering from cancer comprises administering compound (1) orally, preferably as a tablet, twice daily in a daily dose of 540 mg. Preferably, in these embodiments, each of the two daily administrations is of 270 mg.

In some embodiments, compound (1) is administered orally, preferably as a tablet, twice daily in a daily dose of 600 mg. Alternatively, the method of treating a patient suffering from cancer comprises administering compound (1) orally, preferably as a tablet, twice daily in a daily dose of 600 mg. Preferably, in these embodiments, each of the two daily administrations is of 300 mg.

In a preferred embodiment, it is also possible to combine above defined dose regimes for compound (1) and to alter the daily doses in the course of the treatment. Therefore, it is for instance possible to start the treatment by administering a daily dose of 30 mg (once or twice daily) and to switch the dose to higher or lower daily doses (applied once or twice daily).

The dose regimes described in this paragraph are also applicable in cases, wherein the patient already received one or more systemic anti-cancer treatments or therapies.

### Doses and Dose Regimes of the Solid Dispersion and Pharmaceutical Composition

It is a purpose of the present invention to provide a safe and efficacious dose regime for the administration of the solid dispersion comprising compound (1) in the described dose regime. This dose regime is especially helpful for use in the treatment of cancer. In addition, this dose regime of the solid dispersion comprising compound (1) is especially helpful in a method of treating a patient suffering from cancer. In addition, the dose regime seems also to be suitable as a second or further line treatment, wherein the patient already received in the past one or more kinds of cancer treatments.

It was surprisingly discovered that the use of the solid dispersion comprising compound (1) as described herein in the treatment of cancer in a daily dose of compound (1) of at least 30 mg is safe and efficacious as described above and in the example below.

In preferred embodiments, the solid dispersion is administered in a daily dose of compound (1) of at least 60 mg. Alternatively, the method of treating a patient suffering from cancer described above comprises administering the solid dispersion, wherein compound (1) is administered in a daily dose of at least 60 mg.

In preferred embodiments, the solid dispersion is administered in a daily dose of compound (1) of at least 80 mg. Alternatively, a method of treating a patient suffering from cancer comprises administering the solid dispersion, wherein compound (1) is administered in a daily dose of at least 80 mg.

In preferred embodiments, the solid dispersion is administered in a daily dose of compound (1) of at least 120 mg. Alternatively, the method of treating a patient suffering from cancer comprises administering the solid dispersion, wherein compound (1) is administered in a daily dose of at least 120 mg.

In preferred embodiments, the solid dispersion is administered in a daily dose of compound (1) of at least 180 mg. Alternatively, the method of treating a patient suffering from cancer comprises administering the solid dispersion, wherein compound (1) is administered in a daily dose of at least 180 mg.

In preferred embodiments, the solid dispersion is administered in a daily dose of compound (1) of at least 200 mg. Alternatively, the method of treating a patient suffering from cancer comprises administering the solid dispersion, wherein compound (1) is administered in a daily dose of at least 200 mg.

In preferred embodiments, the solid dispersion is administered in a daily dose of compound (1) of at least 240 mg. Alternatively, the method of treating a patient suffering from cancer comprises administering the solid dispersion, wherein compound (1) is administered in a daily dose of at least 240 mg.

In preferred embodiments, the solid dispersion is administered in a daily dose of compound (1) of at least 300 mg. Alternatively, the method of treating a patient suffering from cancer comprises administering the solid dispersion, wherein compound (1) is administered in a daily dose of at least 300 mg.

In preferred embodiments, the solid dispersion is administered in a daily dose of compound (1) of 30 mg to 600 mg. Alternatively, the method of treating a patient suffering from cancer comprises administering the solid dispersion, wherein compound (1) is administered in a daily dose of 30 mg to 600 mg.

In preferred embodiments, the solid dispersion is administered in a daily dose of compound (1) of 60 mg to 600 mg. Alternatively, the method of treating a patient suffering from cancer comprises administering the solid dispersion, wherein compound (1) is administered in a daily dose of 60 mg to 600 mg.

In preferred embodiments, the solid dispersion is administered in a daily dose of compound (1) of 80 mg to 600 mg. Alternatively, the method of treating a patient suffering from cancer comprises administering the solid dispersion, wherein compound (1) is administered in a daily dose of 80 mg to 600 mg.

In preferred embodiments, the solid dispersion is administered in a daily dose of compound (1) of 120 mg to 600 mg. Alternatively, the method of treating a patient suffering from cancer comprises administering the solid dispersion, wherein compound (1) is administered in a daily dose of 120 mg to 600 mg.

In a further preferred embodiment, the solid dispersion is administered in a daily dose of compound (1) of 30 mg to 300 mg. Alternatively, the method of treating a patient suffering from cancer comprises administering the solid dispersion, wherein compound (1) is administered in a daily dose of 30 mg to 300 mg.

In a further preferred embodiment, the solid dispersion is administered in a daily dose of compound (1) of 60 mg to 300 mg. Alternatively, the method of treating a patient suffering from cancer comprises administering the solid dispersion, wherein compound (1) is administered in a daily dose of 60 mg to 300 mg.

In a further preferred embodiment, the solid dispersion is administered in a daily dose of compound (1) of 80 mg to 300 mg. Alternatively, the method of treating a patient suffering from cancer comprises administering the solid dispersion, wherein compound (1) is administered in a daily dose of 80 mg to 300 mg.

In a further preferred embodiment, the solid dispersion is administered in a daily dose of compound (1) of 120 mg to 300 mg. Alternatively, the method of treating a patient suffering from cancer comprises administering the solid dispersion, wherein compound (1) is administered in a daily dose of 120 mg to 300 mg.

In preferred embodiments, the solid dispersion is administered in a daily dose of compound (1) of 30 mg, 60 mg, 120 mg, 180 mg, 200 mg, 240 mg, 300 mg, 360 mg, 400 mg, 420 mg, 480 mg, 500 mg, 540 mg or 600 mg. Alternatively, the method of treating a patient suffering from cancer comprises administering the solid dispersion, wherein compound (1) is administered in a daily dose of 30 mg, 60 mg, 120 mg, 180 mg, 200 mg, 240 mg, 300 mg, 360 mg, 400 mg, 420 mg, 480 mg, 500 mg, 540 mg or 600 mg.

In preferred embodiments, the solid dispersion is administered in a daily dose of compound (1) of 30 mg, 60 mg, 120 mg, 180 mg, 200 mg, 240 mg, 300 mg, 360 mg, 420 mg, 480 mg, 540 mg or 600 mg. Alternatively, the method of treating a patient suffering from cancer comprises administering the solid dispersion, wherein compound (1) is administered in a daily dose of 30 mg, 60 mg, 120 mg, 180 mg, 200 mg, 240 mg, 300 mg, 360 mg, 420 mg, 480 mg, 540 mg or 600 mg.

In preferred embodiments, the solid dispersion is administered in a daily dose of compound (1) of 30 mg, 60 mg, 120 mg, 180 mg, 200 mg, 240 mg or 300 mg. Alternatively, the method of treating a patient suffering from cancer comprises administering the solid dispersion, wherein compound (1) is administered in a daily dose of 30 mg, 60 mg, 120 mg, 180 mg, 200 mg, 240 mg or 300 mg.

In preferred embodiments, the solid dispersion is administered in a daily dose of compound (1) of 30 mg. Alternatively, the method of treating a patient suffering from cancer comprises administering the solid dispersion, wherein compound (1) is administered in a daily dose of 30 mg.

In preferred embodiments, the solid dispersion is administered in a daily dose of compound (1) of 60 mg. Alternatively, the method of treating a patient suffering from cancer comprises administering the solid dispersion, wherein compound (1) is administered in a daily dose of 60 mg.

In preferred embodiments, the solid dispersion is administered in a daily dose of compound (1) of 120 mg. Alternatively, the method of treating a patient suffering from cancer comprises administering the solid dispersion, wherein compound (1) is administered in a daily dose of 120 mg.

In preferred embodiments, the solid dispersion is administered in a daily dose of compound (1) of 180 mg. Alternatively, the method of treating a patient suffering from cancer comprises administering the solid dispersion, wherein compound (1) is administered in a daily dose of 180 mg.

In preferred embodiments, the solid dispersion is administered in a daily dose of compound (1) of 200 mg. Alternatively, the method of treating a patient suffering from cancer comprises administering the solid dispersion, wherein compound (1) is administered in a daily dose of 200 mg.

In preferred embodiments, the solid dispersion is administered in a daily dose of compound (1) of 240 mg. Alternatively, the method of treating a patient suffering from cancer comprises administering the solid dispersion, wherein compound (1) is administered in a daily dose of 240 mg.

In preferred embodiments, the solid dispersion is administered in a daily dose of compound (1) of 300 mg. Alternatively, the method of treating a patient suffering from cancer comprises administering the solid dispersion, wherein compound (1) is administered in a daily dose of 300 mg.

In preferred embodiments, the solid dispersion is administered in a daily dose of compound (1) of 360 mg. Alternatively, the method of treating a patient suffering from cancer comprises administering the solid dispersion, wherein compound (1) is administered in a daily dose of 360 mg.

In preferred embodiments, the solid dispersion is administered in a daily dose of compound (1) of 400 mg. Alternatively, the method of treating a patient suffering from cancer comprises administering the solid dispersion, wherein compound (1) is administered in a daily dose of 400 mg.

In preferred embodiments, the solid dispersion is administered in a daily dose of compound (1) of 420 mg. Alternatively, the method of treating a patient suffering from cancer comprises administering the solid dispersion, wherein compound (1) is administered in a daily dose of 420 mg.

In preferred embodiments, the solid dispersion is administered in a daily dose of compound (1) of 480 mg. Alternatively, the method of treating a patient suffering from cancer comprises administering the solid dispersion, wherein compound (1) is administered in a daily dose of 480 mg.

In preferred embodiments, the solid dispersion is administered in a daily dose of compound (1) of 500 mg. Alternatively, the method of treating a patient suffering from cancer comprises administering the solid dispersion, wherein compound (1) is administered in a daily dose of 500 mg.

In preferred embodiments, the solid dispersion is administered in a daily dose of compound (1) of 540 mg. Alternatively, the method of treating a patient suffering from cancer comprises administering the solid dispersion, wherein compound (1) is administered in a daily dose of 540 mg.

In preferred embodiments, the solid dispersion is administered in a daily dose of compound (1) of 600 mg. Alternatively, the method of treating a patient suffering from cancer comprises administering the solid dispersion, wherein compound (1) is administered in a daily dose of 600 mg.

Within a preferred embodiment, the solid dispersion is administered once or twice daily.

In a preferred embodiment, the solid dispersion is administered once daily. In a preferred embodiment, the solid dispersion is administered as a single dose within 24 h.

In a preferred embodiment, the solid dispersion is administered twice daily. In a preferred embodiment, the solid dispersion is administered twice within 24 h.

In preferred embodiments, each of the two daily administrations of the solid dispersion corresponds to half the daily dose of compound (1). An easy and error-proof application scheme can be provided by administering the required daily dose of compound (1) in two doses comprising the same amount.

In a further preferred embodiment, the solid dispersion is administered at least for 21 consecutive days. In further preferred embodiments, the solid dispersion is administered for 21 days multiplied by X, wherein X is a natural number equal or larger than 1. It is also possible, that in the overall cancer treatment between the treatment times including administering the solid dispersion a dose-free time-interval is included.

In some embodiments, the solid dispersion is administered once daily in a daily dose of compound (1) of 60 mg, 120 mg, 180 mg, 240 mg, 300 mg, 360 mg, 400 mg, 420 mg, 480 mg, 500 mg, 540 mg or 600 mg or is administered twice daily in a daily dose of compound (1) of 30 mg, 60 mg, 120 mg, 200 mg, 300 mg, 360 mg, 400 mg, 420 mg, 480 mg, 500 mg, 540 mg or 600 mg. Alternatively, the method of treating a patient suffering from cancer comprises administering the solid dispersion once daily, wherein compound (1) is administered in a daily dose of 60 mg, 120 mg, 180 mg, 240 mg, 300 mg, 360 mg, 400 mg, 420 mg, 480 mg, 500 mg, 540 mg or 600 mg or compound (1) is administered twice daily in a daily dose of 30 mg, 60 mg, 120 mg, 200 mg, 300 mg, 360 mg, 400 mg, 420 mg, 480 mg, 500 mg, 540 mg or 600 mg. In some embodiments, the solid dispersion is administered once daily in a daily dose of compound (1) of 60 mg, 120 mg, 180 mg, 240 mg, 300 mg, 360 mg, 420 mg, 480 mg, 540 mg or 600 mg or is administered twice daily in a daily dose of compound (1) of 30 mg, 60 mg, 120 mg, 200 mg, 300 mg, 360 mg, 420 mg, 480 mg, 540 mg or 600 mg. Alternatively, the method of treating a patient suffering from cancer comprises administering the solid dispersion once daily, wherein compound (1) is administered in a daily dose of 60 mg, 120 mg, 180 mg, 240 mg, 300 mg, 360 mg, 420 mg, 480 mg, 540 mg or 600 mg or compound (1) is administered twice daily in a daily dose of 30 mg, 60 mg, 120 mg, 200 mg, 300 mg, 360 mg, 420 mg, 480 mg, 540 mg or 600 mg.

In some embodiments, the solid dispersion is administered once daily in a daily dose of compound (1) of 60 mg, 120 mg, 180 mg, 240 mg or 300 mg or is administered twice daily in a daily dose of compound (1) of 30 mg, 60 mg, 120 mg, 200 mg or 300 mg. Alternatively, the method of treating a patient suffering from cancer comprises administering the solid dispersion once daily, wherein compound (1) is administered in a daily dose of 60 mg, 120 mg, 180 mg, 240 mg or 300 mg or compound (1) twice daily in a daily dose of 30 mg, 60 mg, 120 mg, 200 mg or 300 mg.

In some embodiments, the solid dispersion is administered once daily in a daily dose of compound (1) of 60 mg, 120 mg, 180 mg, 240 mg, 300 mg, 360 mg, 400 mg, 420 mg, 480 mg, 500 mg, 540 mg or 600 mg. Alternatively, the method of treating a patient suffering from cancer comprises administering the solid dispersion once daily, wherein compound (1) is administered in a daily dose of 60 mg, 120 mg, 180 mg, 240 mg, 300 mg, 360 mg, 400 mg, 420 mg, 480 mg, 500 mg, 540 mg or 600 mg.

In some embodiments, the solid dispersion is administered once daily in a daily dose of compound (1) of 60 mg, 120 mg, 180 mg, 240 mg, 300 mg, 360 mg, 420 mg, 480 mg, 540 mg or 600 mg. Alternatively, the method of treating a patient suffering from cancer comprises administering the solid dispersion once daily, wherein compound (1) is administered in a daily dose of 60 mg, 120 mg, 180 mg, 240 mg, 300 mg, 360 mg, 420 mg, 480 mg, 540 mg or 600 mg.

In some embodiments, the solid dispersion is administered once daily in a daily dose of compound (1) of 60 mg, 120 mg, 180 mg, 240 mg or 300 mg. Alternatively, the method of treating a patient suffering from cancer comprises administering the solid dispersion once daily, wherein compound (1) is administered in a daily dose of 60 mg, 120 mg, 180 mg, 240 mg or 300 mg.

In preferred embodiments, the solid dispersion is administered once daily in a daily dose of compound (1) of 60 mg. Alternatively, the method of treating a patient suffering from cancer comprises administering the solid dispersion once daily, wherein compound (1) is administered in a daily dose of 60 mg.

In preferred embodiments, the solid dispersion is administered once daily in a daily dose of compound (1) of 120 mg. Alternatively, the method of treating a patient suffering from cancer comprises administering the solid dispersion once daily, wherein compound (1) is administered in a daily dose of 120 mg.

In preferred embodiments, the solid dispersion is administered once daily in a daily dose of compound (1) of 180 mg. Alternatively, the method of treating a patient suffering from cancer comprises administering the solid dispersion once daily, wherein compound (1) is administered in a daily dose of 180 mg.

In preferred embodiments, the solid dispersion is administered once daily in a daily dose of compound (1) of 240 mg. Alternatively, the method of treating a patient suffering from cancer comprises administering the solid dispersion once daily, wherein compound (1) is administered in a daily dose of 240 mg.

In preferred embodiments, the solid dispersion is administered once daily in a daily dose of compound (1) of 300 mg. Alternatively, the method of treating a patient suffering from cancer comprises administering the solid dispersion once daily, wherein compound (1) is administered in a daily dose of 300 mg.

In preferred embodiments, the solid dispersion is administered once daily in a daily dose of compound (1) of 360 mg. Alternatively, the method of treating a patient suffering from cancer comprises administering the solid dispersion once daily, wherein compound (1) is administered in a daily dose of 360 mg.

In preferred embodiments, the solid dispersion is administered once daily in a daily dose of compound (1) of 400 mg. Alternatively, the method of treating a patient suffering from cancer comprises administering the solid dispersion once daily, wherein compound (1) is administered in a daily dose of 400 mg.

In preferred embodiments, the solid dispersion is administered once daily in a daily dose of compound (1) of 420 mg. Alternatively, the method of treating a patient suffering from cancer comprises administering the solid dispersion once daily, wherein compound (1) is administered in a daily dose of 420 mg.

In preferred embodiments, the solid dispersion is administered once daily in a daily dose of compound (1) of 480 mg. Alternatively, the method of treating a patient suffering from cancer comprises administering the solid dispersion once daily, wherein compound (1) is administered in a daily dose of 480 mg.

In preferred embodiments, the solid dispersion is administered once daily in a daily dose of compound (1) of 500 mg. Alternatively, the method of treating a patient suffering from cancer comprises administering the solid dispersion once daily, wherein compound (1) is administered in a daily dose of 500 mg.

In preferred embodiments, the solid dispersion is administered once daily in a daily dose of compound (1) of 540 mg. Alternatively, the method of treating a patient suffering from cancer comprises administering the solid dispersion once daily, wherein compound (1) is administered in a daily dose of 540 mg.

In preferred embodiments, the solid dispersion is administered once daily in a daily dose of compound (1) of 600 mg. Alternatively, the method of treating a patient suffering from cancer comprises administering the solid dispersion once daily, wherein compound (1) is administered in a daily dose of 600 mg.

In some embodiments, the solid dispersion is administered twice daily in a daily dose of compound (1) of 30 mg, 60 mg, 120 mg, 200 mg, 300 mg, 360 mg, 400 mg, 420 mg, 480 mg, 500 mg, 540 mg or 600 mg. Alternatively, the method of treating a patient suffering from cancer comprises administering the solid dispersion twice daily, wherein compound (1) is administered in a daily dose of 30 mg, 60 mg, 120 mg, 200 mg, 300 mg, 360 mg, 400 mg, 420 mg, 480 mg, 500 mg, 540 mg or 600 mg.

In some embodiments, the solid dispersion is administered twice daily in a daily dose of compound (1) of 30 mg, 60 mg, 120 mg, 200 mg, 300 mg, 360 mg, 420 mg, 480 mg, 540 mg or 600 mg. Alternatively, the method of treating a patient suffering from cancer comprises administering the solid dispersion twice daily, wherein compound (1) is administered in a daily dose of 30 mg, 60 mg, 120 mg, 200 mg, 300 mg, 360 mg, 420 mg, 480 mg, 540 mg or 600 mg. In some embodiments, the solid dispersion is administered twice daily in a daily dose of compound (1) of 30 mg, 60 mg, 120 mg, 200 mg or 300 mg. Alternatively, the method of treating a patient suffering from cancer comprises administering the solid dispersion twice daily, wherein compound (1) is administered in a daily dose of 30 mg, 60 mg, 120 mg, 200 mg or 300 mg.

In preferred embodiments, the solid dispersion is administered twice daily in a daily dose of compound (1) of 30 mg. Alternatively, the method of treating a patient suffering from cancer comprises administering the solid dispersion twice daily, wherein compound (1) is administered in a daily dose of 30 mg. Preferably, in these embodiments, each of the two daily administrations is of 15 mg.

In preferred embodiments, the solid dispersion is administered twice daily in a daily dose of compound (1) of 60 mg. Alternatively, the method of treating a patient suffering from cancer comprises administering the solid dispersion twice daily, wherein compound (1) is administered in a daily dose of 60 mg. Preferably, in these embodiments, each of the two daily administrations is of 30 mg.

In preferred embodiments, the solid dispersion is administered twice daily in a daily dose of compound (1) of 120 mg. Alternatively, the method of treating a patient suffering from cancer comprises administering the solid dispersion twice daily, wherein compound (1) is administered in a daily dose of 120 mg. Preferably, in these embodiments, each of the two daily administrations is of 60 mg.

In preferred embodiments, the solid dispersion is administered twice daily in a daily dose of compound (1) of 200 mg. Alternatively, the method of treating a patient suffering from cancer comprises administering the solid dispersion twice daily, wherein compound (1) is administered in a daily dose of 200 mg. Preferably, in these embodiments, each of the two daily administrations is of 100 mg.

In preferred embodiments, the solid dispersion is administered twice daily in a daily dose of compound (1) of 300 mg. Alternatively, the method of treating a patient suffering from cancer comprises administering the solid dispersion twice daily, wherein compound (1) is administered in a daily dose of 300 mg. Preferably, in these embodiments, each of the two daily administrations is of 150 mg.

In preferred embodiments, the solid dispersion is administered twice daily in a daily dose of compound (1) of 360 mg. Alternatively, the method of treating a patient suffering from cancer comprises administering the solid dispersion twice daily, wherein compound (1) is administered in a daily dose of 360 mg. Preferably, in these embodiments, each of the two daily administrations is of 180 mg.

In preferred embodiments, the solid dispersion is administered twice daily in a daily dose of compound (1) of 400 mg. Alternatively, the method of treating a patient suffering from cancer comprises administering the solid dispersion twice daily, wherein compound (1) is administered in a daily dose of 400 mg. Preferably, in these embodiments, each of the two daily administrations is of 200 mg.

In preferred embodiments, the solid dispersion is administered twice daily in a daily dose of compound (1) of 420 mg. Alternatively, the method of treating a patient suffering from cancer comprises administering the solid dispersion twice daily, wherein compound (1) is administered in a daily dose of 420 mg. Preferably, in these embodiments, each of the two daily administrations is of 210 mg.

In preferred embodiments, the solid dispersion is administered twice daily in a daily dose of compound (1) of 480 mg. Alternatively, the method of treating a patient suffering from cancer comprises administering the solid dispersion twice daily, wherein compound (1) is administered in a daily dose of 480 mg. Preferably, in these embodiments, each of the two daily administrations is of 240 mg.

In preferred embodiments, the solid dispersion is administered twice daily in a daily dose of compound (1) of 500 mg. Alternatively, the method of treating a patient suffering from cancer comprises administering the solid dispersion twice daily, wherein compound (1) is administered in a daily dose of 500 mg. Preferably, in these embodiments, each of the two daily administrations is of 250 mg.

In preferred embodiments, the solid dispersion is administered twice daily in a daily dose of compound (1) of 540 mg. Alternatively, the method of treating a patient suffering from cancer comprises administering the solid dispersion twice daily, wherein compound (1) is administered in a daily dose of 540 mg. Preferably, in these embodiments, each of the two daily administrations is of 270 mg.

In preferred embodiments, the solid dispersion is administered twice daily in a daily dose of compound (1) of 600 mg. Alternatively, the method of treating a patient suffering from cancer comprises administering the solid dispersion twice daily, wherein compound (1) is administered in a daily dose of 600 mg. Preferably, in these embodiments, each of the two daily administrations is of 300 mg.

In some embodiments, the solid dispersion is administered orally, preferably as a tablet, once daily in a daily dose of compound (1) of 60 mg, 120 mg, 180 mg, 240 mg, 300 mg, 360 mg, 400 mg, 420 mg, 480 mg, 500 mg, 540 mg or 600 mg. Alternatively, the method of treating a patient suffering from cancer comprises administering the solid dispersion orally, preferably as a tablet, once daily, wherein compound (1) is administered in a daily dose of 60 mg, 120 mg, 180 mg, 240 mg, 300 mg, 360 mg, 400 mg, 420 mg, 480 mg, 500 mg, 540 mg or 600 mg.

In some embodiments, the solid dispersion is administered orally, preferably as a tablet, once daily in a daily dose of compound (1) of 60 mg, 120 mg, 180 mg, 240 mg, 300 mg, 360 mg, 420 mg, 480 mg, 540 mg or 600 mg. Alternatively, the method of treating a patient suffering from cancer comprises administering the solid dispersion orally, preferably as a tablet, once daily, wherein compound (1) is administered in a daily dose of 60 mg, 120 mg, 180 mg, 240 mg, 300 mg, 360 mg, 420 mg, 480 mg, 540 mg or 600 mg.

In some embodiments, the solid dispersion is administered orally, preferably as a tablet, once daily in a daily dose of compound (1) of 60 mg, 120 mg, 180 mg, 240 mg or 300 mg. Alternatively, the method of treating a patient suffering from cancer comprises administering the solid dispersion orally, preferably as a tablet, once daily, wherein compound (1) is administered in a daily dose of 60 mg, 120 mg, 180 mg, 240 mg or 300 mg.

In some embodiments, the solid dispersion is administered orally, preferably as a tablet, once daily in a daily dose of compound (1) of 60 mg. Alternatively, the method of treating a patient suffering from cancer comprises administering the solid dispersion orally, preferably as a tablet, once daily, wherein compound (1) is administered in a daily dose of 60 mg.

In some embodiments, the solid dispersion is administered orally, preferably as a tablet, once daily in a daily dose of compound (1) of 120 mg. Alternatively, the method of treating a patient suffering from cancer comprises administering the solid dispersion orally, preferably as a tablet, once daily, wherein compound (1) is administered in a daily dose of 120 mg.

In some embodiments, the solid dispersion is administered orally, preferably as a tablet, once daily in a daily dose of compound (1) of 180 mg. Alternatively, the method of treating a patient suffering from cancer comprises administering the solid dispersion orally, preferably as a tablet, once daily, wherein compound (1) is administered in a daily dose of 180 mg.

In some embodiments, the solid dispersion is administered orally, preferably as a tablet, once daily in a daily dose of compound (1) of 240 mg. Alternatively, the method of treating a patient suffering from cancer comprises administering the solid dispersion orally, preferably as a tablet, once daily, wherein compound (1) is administered in a daily dose of 240 mg.

In some embodiments, the solid dispersion is administered orally, preferably as a tablet, once daily in a daily dose of compound (1) of 300 mg. Alternatively, the method of treating a patient suffering from cancer comprises administering the solid dispersion orally, preferably as a tablet, once daily, wherein compound (1) is administered in a daily dose of 300 mg.

In some embodiments, the solid dispersion is administered orally, preferably as a tablet, once daily in a daily dose of compound (1) of 360 mg. Alternatively, the method of treating a patient suffering from cancer comprises administering the solid dispersion orally, preferably as a tablet, once daily, wherein compound (1) is administered in a daily dose of 360 mg.

In some embodiments, the solid dispersion is administered orally, preferably as a tablet, once daily in a daily dose of compound (1) of 400 mg. Alternatively, the method of treating a patient suffering from cancer comprises administering the solid dispersion orally, preferably as a tablet, once daily, wherein compound (1) is administered in a daily dose of 400 mg.

In some embodiments, the solid dispersion is administered orally, preferably as a tablet, once daily in a daily dose of compound (1) of 420 mg. Alternatively, the method of treating a patient suffering from cancer comprises administering the solid dispersion orally, preferably as a tablet, once daily, wherein compound (1) is administered in a daily dose of 420 mg.

In some embodiments, the solid dispersion is administered orally, preferably as a tablet, once daily in a daily dose of compound (1) of 480 mg. Alternatively, the method of treating a patient suffering from cancer comprises administering the solid dispersion orally, preferably as a tablet, once daily, wherein compound (1) is administered in a daily dose of 480 mg.

In some embodiments, the solid dispersion is administered orally, preferably as a tablet, once daily in a daily dose of compound (1) of 500 mg. Alternatively, the method of treating a patient suffering from cancer comprises administering the solid dispersion orally, preferably as a tablet, once daily, wherein compound (1) is administered in a daily dose of 500 mg.

In some embodiments, the solid dispersion is administered orally, preferably as a tablet, once daily in a daily dose of compound (1) of 540 mg. Alternatively, the method of treating a patient suffering from cancer comprises administering the solid dispersion orally, preferably as a tablet, once daily, wherein compound (1) is administered in a daily dose of 540 mg.

In some embodiments, the solid dispersion is administered orally, preferably as a tablet, once daily in a daily dose of compound (1) of 600 mg. Alternatively, the method of treating a patient suffering from cancer comprises administering the solid dispersion orally, preferably as a tablet, once daily, wherein compound (1) is administered in a daily dose of 600 mg.

In some embodiments, the solid dispersion is administered orally, preferably as a tablet, twice daily in a daily dose of compound (1) of 30 mg, 60 mg, 120 mg, 200 mg, 300 mg, 360 mg, 400 mg, 420 mg, 480 mg, 500 mg, 540 mg or 600 mg. Alternatively, the method comprises administering the solid dispersion orally, preferably as a tablet, twice daily, wherein compound (1) is administered in a daily dose of 30 mg, 60 mg, 120 mg, 200 mg, 300 mg, 360 mg, 400 mg, 420 mg, 480 mg, 500 mg, 540 mg or 600 mg.

In some embodiments, the solid dispersion is administered orally, preferably as a tablet, twice daily in a daily dose of compound (1) of 30 mg, 60 mg, 120 mg, 200 mg, 300 mg, 360 mg, 420 mg, 480 mg, 540 mg or 600 mg. Alternatively, the method comprises administering the solid dispersion orally, preferably as a tablet, twice daily, wherein compound (1) is administered in a daily dose of 30 mg, 60 mg, 120 mg, 200 mg, 300 mg, 360 mg, 420 mg, 480 mg, 540 mg or 600 mg.

In some embodiments, the solid dispersion is administered orally, preferably as a tablet, twice daily in a daily dose of 30 mg, 60 mg, 120 mg, 200 mg or 300 mg. Alternatively, the method of treating a patient suffering from cancer comprises administering the solid dispersion orally, preferably as a tablet, twice daily, wherein compound (1) is administered in a daily dose of 30 mg, 60 mg, 120 mg, 200 mg or 300 mg.

In some embodiments, the solid dispersion is administered orally, preferably as a tablet, twice daily in a daily dose of compound (1) of 30 mg. Alternatively, the method of treating a patient suffering from cancer comprises administering the solid dispersion orally, preferably as a tablet, twice daily, wherein compound (1) is administered in a daily dose of 30 mg. Preferably, in these embodiments, each of the two daily administrations is of 15 mg.

In some embodiments, the solid dispersion is administered orally, preferably as a tablet, twice daily in a daily dose of compound (1) of 60 mg. Alternatively, the method of treating a patient suffering from cancer comprises administering the solid dispersion orally, preferably as a tablet, twice daily, wherein compound (1) is administered in a daily dose of 60 mg. Preferably, in these embodiments, each of the two daily administrations is of 30 mg.

In some embodiments, the solid dispersion is administered orally, preferably as a tablet, twice daily in a daily dose of compound (1) of 120 mg. Alternatively, the method of treating a patient suffering from cancer comprises administering the solid dispersion orally, preferably as a tablet, twice daily, wherein compound (1) is administered in a daily dose of 120 mg. Preferably, in these embodiments, each of the two daily administrations is of 60 mg.

In some embodiments, the solid dispersion is administered orally, preferably as a tablet, twice daily in a daily dose of compound (1) of 200 mg. Alternatively, the method of treating a patient suffering from cancer comprises administering the solid dispersion orally, preferably as a tablet, twice daily, wherein compound (1) is administered in a daily dose of 200 mg. Preferably, in these embodiments, each of the two daily administrations is of 100 mg.

In some embodiments, the solid dispersion is administered orally, preferably as a tablet, twice daily in a daily dose of compound (1) of 300 mg. Alternatively, the method of treating a patient suffering from cancer comprises administering the solid dispersion orally, preferably as a tablet, twice daily, wherein compound (1) is administered in a daily dose of 300 mg. Preferably, in these embodiments, each of the two daily administrations is of 150 mg.

In some embodiments, the solid dispersion is administered orally, preferably as a tablet, twice daily in a daily dose of compound (1) of 360 mg. Alternatively, the method of treating a patient suffering from cancer comprises administering the solid dispersion orally, preferably as a tablet, twice daily, wherein compound (1) is administered in a daily dose of 360 mg. Preferably, in these embodiments, each of the two daily administrations is of 180 mg.

In some embodiments, the solid dispersion is administered orally, preferably as a tablet, twice daily in a daily dose of compound (1) of 400 mg. Alternatively, the method of treating a patient suffering from cancer comprises administering the solid dispersion orally, preferably as a tablet, twice daily, wherein compound (1) is administered in a daily dose of 400 mg. Preferably, in these embodiments, each of the two daily administrations is of 200 mg.

In some embodiments, the solid dispersion is administered orally, preferably as a tablet, twice daily in a daily dose of compound (1) of 420 mg. Alternatively, the method of treating a patient suffering from cancer comprises administering the solid dispersion orally, preferably as a tablet, twice daily, wherein compound (1) is administered in a daily dose of 420 mg. Preferably, in these embodiments, each of the two daily administrations is of 210 mg.

In some embodiments, the solid dispersion is administered orally, preferably as a tablet, twice daily in a daily dose of compound (1) of 480 mg. Alternatively, the method of treating a patient suffering from cancer comprises administering the solid dispersion orally, preferably as a tablet, twice daily, wherein compound (1) is administered in a daily dose of 480 mg. Preferably, in these embodiments, each of the two daily administrations is of 240 mg.

In some embodiments, the solid dispersion is administered orally, preferably as a tablet, twice daily in a daily dose of compound (1) of 500 mg. Alternatively, the method of treating a patient suffering from cancer comprises administering the solid dispersion orally, preferably as a tablet, twice daily, wherein compound (1) is administered in a daily dose of 500 mg. Preferably, in these embodiments, each of the two daily administrations is of 250 mg.

In some embodiments, the solid dispersion is administered orally, preferably as a tablet, twice daily in a daily dose of compound (1) of 540 mg. Alternatively, the method of treating a patient suffering from cancer comprises administering the solid dispersion orally, preferably as a tablet, twice daily, wherein compound (1) is administered in a daily dose of 540 mg. Preferably, in these embodiments, each of the two daily administrations is of 270 mg.

In some embodiments, the solid dispersion is administered orally, preferably as a tablet, twice daily in a daily dose of compound (1) of 600 mg. Alternatively, the method of treating a patient suffering from cancer comprises administering the solid dispersion orally, preferably as a tablet, twice daily, wherein compound (1) is administered in a daily dose of 600 mg. Preferably, in these embodiments, each of the two daily administrations is of 300 mg.

In a preferred embodiment, it is also possible to combine above defined dose regimes for the solid dispersion and to alter the daily doses of compound (1) in the course of the treatment. Therefore, it is for instance possible to start the treatment by administering a daily dose of compound (1) 30 mg (once or twice daily) and to switch the dose to higher or lower daily doses (applied once or twice daily).

The doses and dose regimes described in this paragraph are also applicable in cases, wherein the patient already received one or more systemic anti-cancer treatments or therapies.

The doses and dose regimens described in this paragraph are also applicable to the pharmaceutical composition as described herein. Therefore, provided herein are embodiments corresponding to those recited in this section wherein the expression “solid dispersion” is replaced by the expression “pharmaceutical composition”.

### Second or Further Line Administration

It was surprisingly discovered that the use of compound (1), the solid dispersion as described herein or the pharmaceutical composition as described herein following a cancer therapy with at least one systemic anti-cancer therapy agent has the potential to improve or stabilize the clinical outcome.

In a preferred embodiment, at least one additional therapeutic agent is administered in a line of treatment prior to or before administration of compound (1) in the doses and dose regimes described herein. Alternatively, the method of treating a patient suffering from cancer as described above comprises administering compound (1) in the doses and dose regimes described herein in a line of treatment after or following the administration of at least one additional therapeutic agent.

In a preferred embodiment, at least one additional therapeutic agent is administered in a line of treatment prior to or before administration of the solid dispersion as described herein or the pharmaceutical composition as described herein in the doses and dose regimes described herein.

Alternatively, the method of treating a patient suffering from cancer as described above comprises administering the solid dispersion as described herein or the pharmaceutical composition as described herein in the doses and dose regimes described herein in a line of treatment after or following the administration of at least one additional therapeutic agent.

In a preferred embodiment, the additional therapeutic agent administered in a line of treatment prior to the administration of compound (1), the solid dispersion as described herein or the pharmaceutical composition as described herein is selected from the group consisting of chemotherapy and systemic anti-cancer therapy agents. The additional therapeutic agent can be used in the treatment of cancer besides the administration of compound (1).

In a preferred embodiment, a systemic anti-cancer therapy agent is administered in a line of treatment prior to or before compound (1). In particular, one or more systemic anti-cancer therapy agent(s) can be administered in a line of treatment prior to compound (1). In other words, it is preferred that compound (1) is used to treat cancer in a line of therapy following or after administration of a line of therapy comprising one or more systemic anti-cancer therapy agent(s). These systemic anti-cancer therapy agents can be administered as separate lines of treatment or in combination with each other in the same line. Additionally or alternatively, the systemic anti-cancer agent(s) administered in a line of treatment prior to compound (1) can be used in combination with any other anti-cancer therapy different from compound (1), whether systemic or not. This means that compound (1) can be administered as second (in case that only one line of treatment comprising at least one systemic anti-cancer therapy is administered prior to compound (1)) or further (in case that more than one line of treatment comprising at least one systemic anti-cancer therapy is administered prior to compound (1)) line treatment. In this embodiment, compound (1) is preferably administered in the solid dispersion, the doses and dosing regimes described herein.

In a preferred embodiment, a systemic anti-cancer therapy agent is administered in a line of treatment prior to or before the solid dispersion as described herein or the pharmaceutical composition as described herein. In particular, one or more systemic anti-cancer therapy agent(s) can be administered in a line of treatment prior to the solid dispersion as described herein or the pharmaceutical composition as described herein. In other words, it is preferred that the solid dispersion as described herein or the pharmaceutical composition as described herein is used to treat cancer in a line of therapy following or after administration of a line of therapy comprising administration of one or more systemic anti-cancer therapy agent(s). These systemic anti-cancer therapy agents can be administered as separate lines of treatment or in combination with each other in the same line. Additionally or alternatively, the systemic anti-cancer agent(s) administered in a line of treatment prior to the solid dispersion as described herein or the pharmaceutical composition as described herein can be used in combination with any other anti-cancer therapy different from compound (1), whether systemic or not. This means that the solid dispersion as described herein or the pharmaceutical composition as described herein can be administered as second (in case that only one line of treatment comprising at least one systemic anti-cancer therapy is administered prior to the solid dispersion as described herein or the pharmaceutical composition as described herein) or further (in case that more than one line of treatment comprising at least one systemic anti-cancer therapy is administered prior to the solid dispersion as described herein or the pharmaceutical composition as described herein) line treatment. In this embodiment, the solid dispersion can be as defined herein in any aspect or embodiment. In this embodiment, compound (1) is preferably administered in the doses and dosing regimes described herein.

In this context, the terms “after” and “following” mean that the additional therapeutic agent, especially the chemotherapy agent or the systemic anti-cancer therapy agent, is administered in a first or prior line of therapy during a first time period, for example over the course of a few hours, days or one or more weeks, using one or more doses, and is followed by administration of compound (1), optionally as the solid dispersion or pharmaceutical composition as described herein, in a second or further line of therapy during a second time period, for example over the course of a few hours, days or one or more weeks, using one or more doses, provided there is no overlap between the first and second time periods.

The systemic anti-cancer therapy agent or chemotherapy agent and compound (1) are not administered on the same day. In particular, administration of a single systemic anti-cancer therapy agent or chemotherapy agent is not re-started with the same dosing (of said single systemic anti-cancer therapy agent or chemotherapy agent) once compound (1) is administered.

The systemic anti-cancer therapy agent or chemotherapy agent and the solid dispersion as described herein or the pharmaceutical composition as described herein are not administered on the same day. In particular, administration of a single systemic anti-cancer therapy agent or chemotherapy agent is not re-started with the same dosing (of said single systemic anti-cancer therapy agent or chemotherapy agent) once the solid dispersion as described herein or the pharmaceutical composition as described herein is administered.

The terms “after” and “following” do not require that compound (1), the solid dispersion as described herein or the pharmaceutical composition as described herein is administered in a line of therapy directly after or directly following the line of therapy with the systemic anti-cancer therapy agent(s). Therefore, it may be that another line of therapy is administered between the systemic anti-cancer therapy agent or chemotherapy and compound (1), optionally formulated in the solid dispersion or pharmaceutical composition as described herein, so long as the systemic anti-cancer therapy or chemotherapy is administered in a line of treatment before compound (1), optionally formulated in the solid dispersion or pharmaceutical composition as described herein. Preferably, the systemically detectable doses of the anti-cancer therapy agent in the previously performed anti-cancer therapy are below an established therapeutically effective amount before compound (1) is administered. In a further preferred embodiment, the selected daily dose and the daily application schedule of compound (1), optionally formulated in the solid dispersion or pharmaceutical composition as described herein, can be chosen also as a function of one or all pre-treatments with a systemic anti-cancer therapy agent.

The skilled person will recognize that the terms “before”, “prior”, “after” and “following” are used herein to refer to different and/or separate lines of therapy. In other words, any reference to the administration of compound (1), the solid dispersion or the pharmaceutical composition after or following systemic anti-cancer therapy or chemotherapy corresponds to a reference to the administration of compound (1), the solid dispersion or the pharmaceutical composition as second or further line after administration of systemic anti-cancer therapy or chemotherapy, even when this is not explicitly stated.

As used herein, “second or further line” and grammatical variants thereof have the meaning known in the art. In particular, “second or further line” and grammatical variants thereof can refer to the administration of compound (1), optionally formulated in the solid dispersion or pharmaceutical composition as described herein, after or following a first or prior line of therapy that has failed, stopped working, decreased efficacy, intolerable side effects or been only partially successful, according to the judgment of the attending physician, especially wherein the first or prior line of therapy is never again administered to the patient. The expression “second or further line administration” can be read as “second line administration or further line administration”.

As used herein, the “systemic anti-cancer therapy agent” can be present and can be administered in the form of only a single drug compound or single active ingredient. The agent may also be present and administered in the form of a combination of two or more drug compounds or active ingredients. Drug compounds may include small or large molecules, chemical elements, like Pt, biologics and combinations thereof.

As used herein, the term “systemic anti-cancer therapy” includes the administration of at least one systemic anti-cancer therapy agent, alone or in combination with another drug compound or active ingredient.

As used herein, the “chemotherapy agent” or “chemotherapeutic agent” can be present and can be administered in the form of only a single drug compound or single active ingredient. The agent may also be present and administered in the form of a combination of two or more drug compounds or active ingredients.

As used herein, the term “chemotherapy” includes the administration of at least one chemotherapeutic agent, alone or in combination with another drug compound or active ingredient.

Preferably, chemotherapy is administered systemically, i.e. it is systemic chemotherapy.

In a preferred embodiment, the chemotherapy or systemic anti-cancer therapy agent is selected from the group consisting of platinum-based chemotherapy, anti-HER2 antibody-drug conjugates, taxanes, anti-metabolites, immunotherapeutic agents and combinations thereof. Preferably, the term “combinations thereof” might include the separate administration of two or more members of that list in different time intervals or at different points in time, as different lines of therapy or the administration of two or more members of that list in combination in the same line of therapy. The combination might for instance include a fist line treatment including a platinum-based chemotherapy agent and, later on, a second line treatment based on an ADC, preferably an anti-HER2-ADC, or vice versa, prior to a treatment based on compound (1). The combination can be a planned sequence or can be performed as a function of the treatment outcome.

Preferred platinum-based chemotherapy includes Carboplatin and/or Cisplatin. Preferred anti-HER2 antibody-drug conjugates include: trastuzumab deruxtecan and/or trastuzumab emtansine. Preferred taxanes include Docetaxel and/or Paclitaxel. Preferred anti-metabolites include: Gemcitabine, Pemetrexed and/or Tegafur. Preferred immunotherapeutic agents include: Pembrolizumab, Durvalumab, Atezolizumab, Nivolumab, Ipilimumab, Tremelimumab, and/or Ramucirumab.

The term “antibody drug conjugate” (also abbreviated herein as “ADC”), as used herein, is well known in the art and describes a group of therapeutics that combine the specificity of tumor-targeting binders, such as e.g. antibodies, with the potency of highly cytotoxic agents. ADCs are well known in the art and have been reviewed, for example, in Dumontet et al. 2023 (Dumontet, C., Reichert, J. M., Senter, P. D. et al. Antibody-drug conjugates come of age in oncology. Nat Rev Drug Discov 22, 641-661 (2023)).

In particular, “anti-HER2 antibody-drug conjugate”, as used herein, refers to an ADC whose tumor-targeting binder is an antibody directed to, targeting and/or binding HER2, such as trastuzumab.

In a preferred embodiment, the chemotherapy or systemic anti-cancer therapy agent is selected from the group consisting of: Carboplatin, Cisplatin, trastuzumab deruxtecan, trastuzumab emtansine, Pemetrexed, Docetaxel, Paclitaxel, Gemcitabine, Pembrolizumab, Durvalumab, Tremelimumab, Ramucirumab, Atezolizumab, Tegafur, Nivolumab and Ipilimumab.

In a further preferred embodiment, compound (1) as defined below

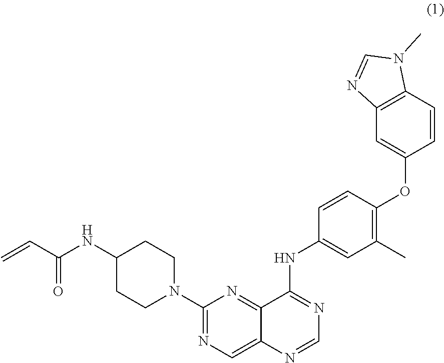 [SMILES: C=CC(=O)NC1CCN(c2ncc3ncnc(Nc4ccc(Oc5ccc6c(c5)ncn6C)c(C)c4)c3n2)CC1]

is for use in the treatment of cancer, wherein compound (1) is administered in a line of treatment following administration of chemotherapy or a systemic anti-cancer therapy agent. Alternative, in a preferred embodiment a method of treating a patient suffering from cancer comprises administering compound (1), wherein compound (1) is administered in a line of treatment following administration of chemotherapy or a systemic anti-cancer therapy agent. Preferably, in these embodiments, compound (1) is administered according to the doses and dose regimes described hereinabove. In addition, or in alternative, in these embodiments, the cancer and/or the systemic anti-cancer therapy agent may be as herein defined. Preferably, the systemic anti-cancer therapy agent is a specific anti HER2 systemic anti-cancer therapy agent, e.g. an anti-HER2 antibody-drug conjugate.

In a preferred embodiment, compound (1) can be used in the treatment of cancer at least 21 days after the last day of administering the chemotherapy or systemic anti-cancer therapy agent. Alternatively, in a preferred embodiment, the method of treating a patient suffering from cancer comprises administering compound (1), wherein compound (1) is administered at least 21 days after the last day of administering the chemotherapy or systemic anti-cancer therapy agent.

In a further preferred embodiment, the solid dispersion comprising compound (1) as defined herein or a pharmaceutically acceptable salt thereof and a pharmaceutically acceptable dispersion carrier is for use in the treatment of cancer, wherein the solid dispersion is administered in a line of treatment following administration of chemotherapy or a systemic anti-cancer therapy agent. Alternative, in a preferred embodiment a method of treating a patient suffering from cancer comprises administering the solid dispersion comprising compound (1) as defined herein or a pharmaceutically acceptable salt thereof and a pharmaceutically acceptable dispersion carrier, wherein the solid dispersion is administered in a line of treatment following administration of chemotherapy or a systemic anti-cancer therapy agent. Preferably, in these embodiments, the solid dispersion is administered according to the doses and dose regimes described hereinabove. In addition, or in alternative, in these embodiments, the cancer and/or the systemic anti-cancer therapy agent may be as herein defined. Preferably, the systemic anti-cancer therapy agent is a specific anti HER2 systemic anti-cancer therapy agent, e.g. an anti-HER2 antibody-drug conjugate.

In a preferred embodiment, the solid dispersion comprising compound (1) as defined herein or a pharmaceutically acceptable salt thereof and a pharmaceutically acceptable dispersion carrier can be used in the treatment of cancer at least 21 days after the last day of administering the chemotherapy or systemic anti-cancer therapy agent. Alternatively, in a preferred embodiment, the method of treating a patient suffering from cancer comprises administering the solid dispersion comprising compound (1) as defined herein or a pharmaceutically acceptable salt thereof and a pharmaceutically acceptable dispersion carrier, wherein compound (1) is administered at least 21 days after the last day of administering the chemotherapy or systemic anti-cancer therapy agent.

### Process of Preparing a Solid Dispersion

Solid dispersions of the invention can be prepared from any process known in the art for this purpose, for example as disclosed in S. V. Bhujbal et al., Acta Pharmaceutica Sinica B 2021; 11(8):2505e2536, which is herein incorporated by reference. According to the invention, solid dispersions are generally prepared by dissolving an active substance and a pharmaceutically acceptable dispersion carrier in a solvent or mixture of solvents to form a feed solution, and then the solvent is removed from the feed solution, such as by spray-drying, to form the solid dispersion.

In an embodiment, a process of preparing the solid dispersion as described herein is provided, the process comprising the steps of:
a) providing a mixture of compound (1) or a pharmaceutically acceptable salt thereof and a pharmaceutically acceptable dispersion carrier and adding a solvent to obtain a solution or a suspension; and
b) removing the solvent from the solution or the suspension to form the solid dispersion as described herein.

This process may further comprise the step of drying the solid dispersion obtained in step b).

In an embodiment, a process of preparing the solid dispersion as described herein is provided, the process comprising the steps of:
a) providing a solution or suspension comprising compound (1) or a pharmaceutically acceptable salt thereof, a pharmaceutically acceptable dispersion carrier and at least one solvent; and
b) removing the solvent from the solution or the suspension to form the solid dispersion as described herein; and
c) optionally, drying the solid dispersion obtained in b).

The solution or suspension of step a) according to any of the above-described processes can be referred to as a feed solution.

In embodiments, the removing of the solvent in step b) of the above defined processes is carried out by spray-drying, freeze drying, rotary evaporation, distillation, drum drying and/or vacuum drying. In a preferred embodiment, the removing of the solvent in step b) is carried out by spray-drying.

The term “spray drying” as used herein is used conventionally and broadly and generally refers to any process that involves the atomization of a solution, suspension, slurry, or emulsion containing one or more components of the desired product into droplets by spraying followed by the rapid evaporation of the sprayed droplets into solid powder by hot air at a certain temperature and pressure. Spray drying is a process known to a person skilled in the art.

Spray drying is generally performed by dissolving compound (1) and the pharmaceutically acceptable dispersion carrier in a solvent to prepare a feed solution. The feed solution may be pumped through an atomizer into a drying chamber. The feed solution can be atomized by conventional means known in the art, such as a two-fluid sonicating nozzle, a pressure nozzle, a rotating nozzle and a two-fluid non-sonicating nozzle. Then, the solvent is removed in the drying chamber to form the solid dispersion. A typical drying chamber uses hot gases, such as forced air, nitrogen, nitrogen-enriched air, or argon to dry particles. The size of the drying chamber may be adjusted to achieve particle properties or throughput.

Although the solid dispersion is preferably prepared by conventional spray drying techniques, other techniques known in the art may be used, such as melt extrusion, freeze drying, rotary evaporation, co-precipitation, KinetiSol® Dispersing Technology (KSD), fluidized bed technology, drum drying, vacuum drying or other solvent removal processes.

The processes described above of preparing the solid dispersion as described herein may comprise an additional step between steps a) and b) of spraying the solution or suspension obtained in step a) onto inert excipient cores. This process belongs to fluid bed technology, in particular fluid bed granulation technology.

In an embodiment, a process of preparing the solid dispersion as described herein is provided, the process comprising the steps of:
(a) providing a solution or suspension comprising compound (1) or a pharmaceutically acceptable salt thereof, a pharmaceutically acceptable dispersion carrier and at least one solvent; and
(a′) spraying the solution or suspension provided in (a) onto inert excipient cores; and
(b′) removing the solvent from the inert excipient cores; and
(c′) optionally, drying the excipient cores comprising the solid dispersion obtained in (c′).

The spraying in step (a′) may be performed in a fluidized bed coater e.g. as top spray, bottom spray, Wurster, tangential or side rotor spray.

Any solvent or mixture of solvents where compound (1) at least partially dissolves can be used. Examples of suitable solvents that can be used individually or as mixtures include water, alcohols, such as methanol (“MeOH”), ethanol (“EtOH”), n-propanol, isopropanol and butanol such as n-butanol, 2-butanol, isobutanol and tert-butanol; ketones, such as acetone, methyl ethyl ketone and methyl isobutyl ketone; esters, such as methyl acetate, ethyl acetate and propyl acetate, isopropyl acetate, n-butyl acetate and isobutyl acetate; and various other solvents, such as dichloromethane (DCM), chloroform, tetrahydrofuran, acetonitrile, toluene and 1,1,1-trichloroethane. In an embodiment, the solvent referred to in any of the above described processes and embodiments thereof is selected from the group consisting of water, alcohols, ketones, esters, dichloromethane, chloroform, tetrahydrofuran, acetonitrile, toluene, 1,1,1-trichloroethane and mixtures thereof. In an embodiment, the solvent referred to in any of the above described processes and embodiments thereof is selected from the group consisting of alcohols (in particular methanol, ethanol, n-propanol, isopropanol and butanol such as n-butanol, 2-butanol, isobutanol and tert-butanol), ketones (in particular acetone, methyl ethyl ketone and methyl isobutyl ketone), esters (in particular methyl acetate, ethyl acetate and propyl acetate, isopropyl acetate, n-butyl acetate and isobutyl acetate), dichloromethane (DCM), tetrahydrofuran, acetonitrile, toluene and 1,1,1-trichloroethane. Mixtures of solvents with water may also be used.

In embodiments, said solvent is a mixture of dichloromethane (DCM) and methanol (MeOH). The relative amounts of DCM and MeOH in the mixture may vary. Preferably, the mixture comprises at least 25 wt % MeOH based on a total weight of 100 wt % of the mixture. In embodiments, the mixture comprises an excess of DCM. Still preferably, the weight:weight ratio of DCM:MeOH ranges from 25:75 to 95:5 (w/w). Preferably, DCM and MeOH are in a weight:weight ratio of approximately 25:75, 50:50, 70:30, 75:25, 80:20, 85:15 or 90:10. A solvent mixture of DCM:MeOH in a ratio of approximately 90:10 (w/w) was advantageously found to enable higher throughput for spray-drying.

In embodiments, the concentration of solids in the feed solution (in particular the suspension or solution as defined in step a) above) is in the range of from about 1 to 20 wt %, based on a total weight of 100 wt % of the feed solution. Preferably, the concentration of solids in the feed solution is in the range of from about 5 to 15 wt %, more preferably of from about 8 to 12 wt % based on a total weight of 100 wt % of the feed solution. For example, the concentration of solids in the feed solution is about 8 wt % or 10 wt %, based on a total weight of 100 wt % of the feed solution.

After removal of the solvent by spray drying, the obtained solid dispersion is optionally subjected to a drying process in order to reduce residual solvent content. In embodiments, drying is performed at a temperature in the range of from about room temperature to 100° C., preferably of from about 30 to 60° C., more preferably of from about 35 to 45° C. For example, the drying is performed at a temperature of about 40° C. In other embodiments, the drying is performed at ambient pressure and/or under reduced pressure. For example, the drying is performed at ambient pressure or at a pressure of about 900 mbar or less, more preferably of about 100 mbar or less and most preferably of about 50 mbar or less, such as about 20 mbar or less. In still other embodiments, the drying is performed for a period in the range of from about 6 to 72 hours, preferably of from about 12 to 48 hours.

Another aspect relates to a solid dispersion obtainable by a process comprising the steps of:
a) providing a mixture of compound (1) as defined above or a pharmaceutically acceptable salt thereof and a pharmaceutically acceptable dispersion carrier and adding a solvent to obtain a solution or a suspension; and
b) removing the solvent from the solution or the suspension to form the solid dispersion, preferably wherein the removing of the solvent in step b) is carried out by spray-drying.

Another aspect relates to a solid dispersion obtainable by a process comprising the steps of:
a) providing a solution or suspension comprising compound (1) or a pharmaceutically acceptable salt thereof, a pharmaceutically acceptable dispersion carrier and at least one solvent; and
b) removing the solvent from the solution or the suspension to form the solid dispersion as described herein; and
c) optionally, drying the solid dispersion obtained in b).

Another aspect relates to a solid dispersion obtainable by a process comprising the steps of:
(a) providing a solution or suspension comprising compound (1) or a pharmaceutically acceptable salt thereof, a pharmaceutically acceptable dispersion carrier and at least one solvent; and
(a′) spraying the solution or suspension provided in (a) onto inert excipient cores; and
(b′) removing the solvent from the inert excipient cores; and
(c′) optionally, drying the excipient cores comprising the solid dispersion obtained in (c′).

In these aspects relating to a solid dispersion obtainable by a process, the process steps can be performed as described above with reference to the processes of preparing the solid dispersion. Pharmaceutical compositions, such as tablets, preferably film-coated tablets, can be manufactured according to conventional methods known to a skilled person. In embodiments, the manufacturing process can comprise the steps of 1) manufacturing a solid dispersion such as by spray-drying as described herein, 2) dry granulating of the solid dispersion with one or more suitable excipient(s), 3) blending the granules with suitable disintegrant(s) and/or lubricant(s) and/or glidants, 4) compressing the blend into tablet cores, and 5) optionally film-coating the tablet cores.

In the present invention, any aspect or embodiment referring to a feature (e.g. compound (1) being amorphous in the solid dispersion) can be combined to any one or more aspect(s) or embodiment(s) referring to (an)other feature(s) (e.g. the weight ratio of compound (1): the pharmaceutically acceptable dispersion carrier in the solid dispersion being 1:1 and/or the pharmaceutically acceptable dispersion carrier being HPMCAS), to provide further aspects or embodiments of the invention, e.g.
1) the solid dispersion comprising compound (1) or a pharmaceutically acceptable salt thereof and a pharmaceutically acceptable dispersion carrier, wherein compound (1) is amorphous and the weight ratio of compound (1): the pharmaceutically acceptable dispersion carrier in the solid dispersion is 1:1;
2) the solid dispersion comprising compound (1) or a pharmaceutically acceptable salt thereof and a pharmaceutically acceptable dispersion carrier, wherein compound (1) is amorphous and the pharmaceutically acceptable dispersion carrier is HPMCAS;
3) the solid dispersion comprising compound (1) or a pharmaceutically acceptable salt thereof and a pharmaceutically acceptable dispersion carrier, wherein compound (1) is amorphous, the weight ratio of compound (1): the pharmaceutically acceptable dispersion carrier in the solid dispersion is 1:1 and the pharmaceutically acceptable dispersion carrier is HPMCAS.

The expressions “as defined herein”, “as disclosed herein”, “as described herein”, “as used herein” and variants thereof at each instance they occur include all aspects, embodiments, subaspects, subembodiments, etc. of the feature or term they refer to.

In some embodiments, numerical values are cited, sometimes as part of a range. Said numerical values should be taken as approximate values, even when the terms “approximately” or “about” are not explicitly cited.

Unless otherwise defined, the technical and scientific terms used herein have the same meaning as commonly understood by one of ordinary skill in the art to which this invention belongs.

The examples which follow serve to illustrate the invention in more detail but do not constitute a limitation thereof.

### Example 1—Manufacture of Solid Dispersions Comprising Compound (1) and Different Dispersion Polymers

In this Example, solid dispersions were prepared containing 25 wt % or 50 wt % compound (1) and 75 wt % or 50 wt %, respectively, of dispersion carriers HPMCAS-M (Shin-Etsu AQOAT), PVP-VA, Eudragit® L100 or HPMC HME 15LV.

The solid dispersions of this Example can be prepared according to the following protocol: the solid dispersion is spray dried from a spray solution composition comprising compound (1), the dispersion carrier and a DCM:MeOH (1:1 (w/w)) solvent system with a solids content of 8 wt % total solids. Solid dispersions are manufactured using a Procept 4M8TRX spray drier with 2-fluid nozzle type and 1.0 mm/1.0 mm nozzle cap/tip dimension, an inlet temperature of 85-90° C., an outlet temperature of 45-50° C., atomization at 3.0 bar, drying gas air flow rate of 0.50 m³/min, and a solution feed rate of approx. 15 g/min. Secondary drying of the dispersion is done in a vacuum dryer of tray dryer type in collection vessels, at 40° C. for 22.5 hours.

The spray drying yield results obtained following the protocol from the previous paragraph are summarized in Table 1, where gA stands for gram API (active pharmaceutical ingredient, i.e. compound (1)).

TABLE 1
Batch sizes and yield for solid dispersions of compound (1) with dispersion carriers
HPMCAS-M, PVP-VA, Eudragit ® L100 or HPMC HME 15LV

| | Batch size | |
|---|---|---|
| Formulation | [gA] | Yield [%]/gA |
| 25 wt %:75 wt % compound (1):HPMCAS-M | 12 gA | 85% (10.2 g) |
| 50 wt %:50 wt % compound (1):HPMCAS-M | 0.25 gA | 64% (9.5 gA) |
| 25 wt %:75 wt % compound (1):PVP-VA | 12 gA | 79% (9.5 gA) |
| 50 wt %:50 wt % compound (1):PVP-VA | 0.25 gA | 72% (0.18 gA) |
| 25 wt %:75 wt % compound (1):Eudragit ® L100 | 0.25 gA | 76% (0.19 gA) |
| 50 wt %:50 wt % compound (1):Eudragit ® L100 | 0.25 gA | 60% (0.15 gA) |
| 25 wt %:75 wt % compound (1):HPMC HME 15LV | 0.25 gA | 52% (0.13 g) |
| 50 wt %:50 wt % compound (1):HPMC HME 15LV | 0.25 gA | 32% (0.08 gA) |

### Example 2—Characterization of Solid Dispersions by X-Ray Powder Diffraction (XRPD) and Modulated Differential Scanning Calorimetry (mDSC)

### 2.1 X-Ray Powder Diffraction (XRPD)

XRPDs can be obtained according to the following protocol: XRPD analysis is done with a Rigaku Miniflexx 600 diffractometer. An amount of approx. 10 mg of samples of the solid dispersions of compound (1) with dispersion carriers, e.g. with HPMCAS-M, PVP-VA, Eudragit® L100 or HPMC HME 15LV as in Example 1, is placed onto a zero-background sample disk and placed into the auto sampler of the Rigaku Miniflex 600. Samples are analyzed using the instrument parameters described in Table 2 below.

TABLE 2
Summary of XRPD collection parameters

| Parameter | Value |
|---|---|
| ⊖/2-⊖ | 3-60° |
| Radiation source | Cu-Kα1,2 (1.54184 Å) |
| Spin | On |
| Step size | 0.02° |
| Step rate | 2.0 deg/min |
| Voltage | 40 kV |
| Current | 15 mA |
| Divergent slit | 1.0 mm |
| Holder | Zero-Background |

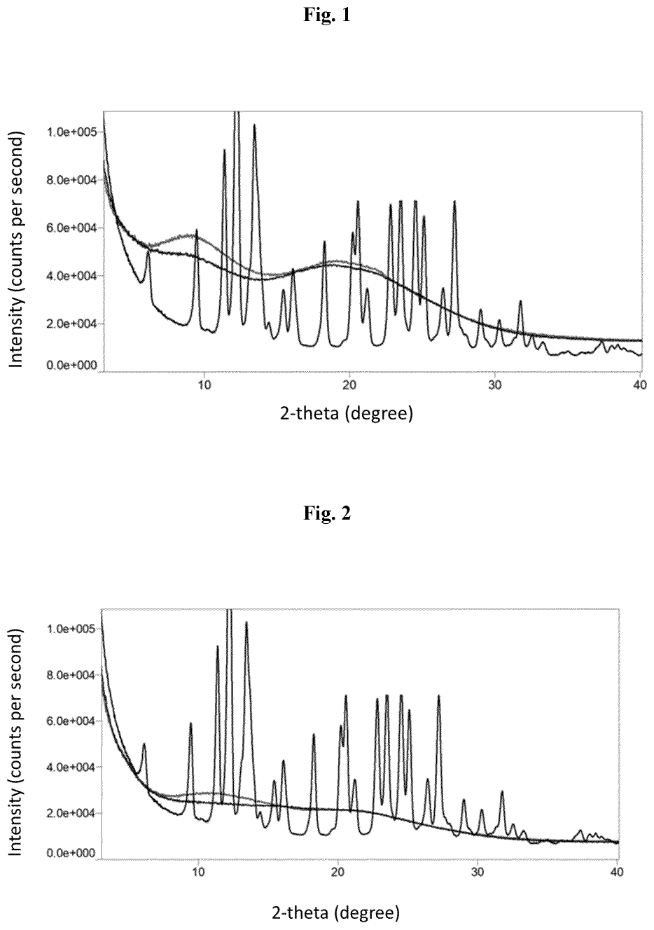

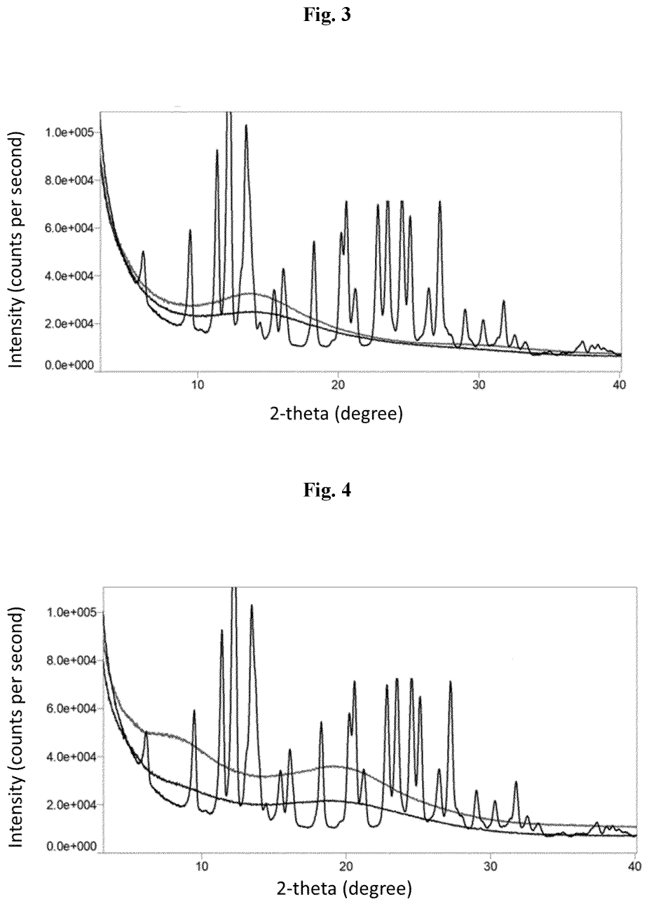

The XRPDs obtained following the protocol from the previous paragraph with the various solid dispersions prepared under Example 1 are displayed in FIGS. 1 to 4. Comparing them against the XRPD of crystalline compound (1) in the same Figures indicates the absence of crystalline material in the samples. Specifically, the XRPDs of the solid dispersion of 25 wt % or 50 wt % compound (1) and dispersion carriers HPMCAS-M, PVP-VA, Eudragit® L100 or HPMC HME 15LV exhibited a lack of sharp peaks and the presence of amorphous halos. The lack of sharp diffraction peaks is indicative that the solid dispersions are consistent with an amorphous form of compound (1).

2.2 Modulated Differential Scanning Calorimetry (mDSC)

The solid dispersions of compound (1) with dispersion carriers HPMCAS-M, PVP-VA, Eudragit® L100 or HPMC HME 15LV of Example 1 were characterized by mDSC to determine the glass transition temperature (Tg).

mDSC can be performed according to the following protocol: mDSC analysis is done on a Thermal Analysis DSC 2500 with a Thermal Analysis Refrigerated Cooling System 90. An amount of 2-5 mg of samples is placed into a non-hermetic pan. A Tzero non-hermetic lid is affixed onto the pan and samples are analyzed in modulated mode in a scan range of 20 to 250° C. (unless otherwise specified) with a modulation amplitude of 1° C./min and a ramp rate (heating rate) of 3.0° C./min.

A summary of the glass transition temperatures (Tg) obtained following the protocol from the previous paragraph is presented in Table 3.

TABLE 3
Summary of glass transition temperatures (Tg) measured for the
solid dispersions of compound (1) with HPMCAS-M, PVP-VA,
Eudragit ® L100 and HPMC HME 15LV of Example 1

| Formulation | Tg |
|---|---|
| amorphous compound (1) | 156° C. |
| 25 wt %:75 wt % compound (1):HPMCAS-M | 115° C. |
| 50 wt %:50 wt % compound (1):HPMCAS-M | 116° C. |
| 25 wt %:75 wt % compound (1):PVP-VA | 113° C |
| 50 wt %:50 wt % compound (1):PVP-VA | 117° C. |
| 25 wt %:75 wt % compound (1):Eudragit ® L100 | 188° C. |
| 50 wt %:50 wt % compound (1):Eudragit ® L100 | 175° C. |
| 25 wt %:75 wt % compound (1):HPMC HME 15LV | 92° C. |
| 50 wt %:50 wt % compound (1):HPMC HME 15LV | 99° C. |

As can be taken from Table 3, all solid dispersions exhibited a single glass transition temperature, indicating a homogenous dispersion without evidence of phase separation. Glass transition temperature values trended as expected with respect to polymer and loading with compound (1). For Eudragit® L100 with glass transition temperatures greater than amorphous compound (1), the glass transition temperature decreased with increasing drug loading. For polymers HPMCAS-M, PVP-VA, and HPMC HME 15LV with glass transition temperatures below that of compound (1), glass transition temperature increased with increasing drug loading. All solid dispersions provided a glass transition temperature that is sufficiently high.

### Example 3—Manufacture of Solid Dispersions Using Different Process Parameters

Solid dispersions of compound (1) were tested for different spray-drying conditions, such as solvents, inlet temperature, and spray drying parameters.

### 3.1 Solvent Selection

The solubility of compound (1) was measured in solvent blends of dichloromethane (DCM):methanol.

To do so, the following protocol can be followed: a solution of compound (1) is prepared at a high concentration, then diluted with solvent until compound (1) dissolves or until the concentration dropped below 1 wt %. Solubility is determined by visual observation.

The solubility data of compound (1) obtained following this protocol are shown in Table 4 below.

TABLE 4
Organic solubility screening results for compound (1)

| | |
|---|---|
| | Solubility |
| Solvent | [wt %] |

DCM:methanol ratios

| | |
|---|---|
| 90:10 | >17.1 |
| 80:20 | >18.7 |
| 70:30 | >16.8 |

Therefore, a solvent blend of 90:10 DCM:methanol provides high solubility of compound (1) and has the highest potential throughput due to a lower concentration of methanol in the solvent system compared to DCM:methanol solvent blends with higher methanol ratio.

### 3.2 Inlet Temperature

Solid dispersions comprising 50 wt % compound (1) and 50 wt % HPMCAS-M or 25 wt % compound (1) and 75 wt % IPMC HME 15LV were manufactured at a larger scale using lower inlet temperature than in Example 1.

To do so, the following protocol can be followed: the solid dispersions are spray dried from a spray solution composition comprising compound (1) and HPMCAS-M or IPMC HME 15LV using a DCM:MeOH (1:1 (w/w)) solvent system with a solids content of 8 wt % total solids. Solid dispersions are manufactured using a Procept 4M8TRX spray drier with 2-fluid nozzle type and 1.0 mm/1.0 mm nozzle cap/tip dimension, an inlet temperature of 55-70° C., an outlet temperature of 45-50° C., atomization at 3.0 bar, drying gas air flow rate of 0.50 m³/min, and a solution feed rate of approx. 10-15 g/min. Secondary drying of the dispersion is done in a vacuum dryer of tray dryer type in collection vessels, at 40° C. for approx. 24 hours.

The spray drying yield results obtained following this protocol are summarized in Table 5, where gA stands for gram API (active pharmaceutical ingredient, i.e. compound (1)). mDSC results (non-hermetic pan, heating 3° C./min, modulation 1° C./min) are summarized in Table 6.

TABLE 5
Batch sizes and yield for solid dispersions of larger batch size and lower inlet temperature

| | Batch size | |
|---|---|---|
| Formulation | [gA] | Yield [%]/gA |
| 50 wt %:50 wt % compound (1):HPMCAS-M | 12 gA | 67% (8.0 gA) |
| 25 wt %:75 wt % compound (1):HPMC HME 15LV | 15 gA | 73 (10.9 gA) |

TABLE 6
mDSC results

| Formulation | Tg |
|---|---|
| amorphous compound (1) | 156° C. |
| 50 wt %:50 wt % compound (1):HPMCAS-M | 115° C. |
| 25 wt %:75 wt % compound (1):HPMC HME 15LV | 92° C. |

### 3.3 Spray Drying Parameters

The effects of flow rate (=liquid feed rate), dryer outlet temperature, and drying environment on the spray dried solid dispersions were tested. For the tests a composition containing 50 wt % compound (1) and 50 wt % of dispersion carrier HPMCAS-M was used. The composition was spray dried from a spray solution composition comprising compound (1) and HPMCAS-M using a solvent ratio DCM:MeOH of 90:10 (w/w) with a solids content of 10 wt % total solids. Solid dispersions were manufactured using an open loop custom development spray dryer SD-90, with SK 79-16 spray systems nozzle. Liquid feed rates between 291 and 317 g/min and outlet temperatures between 35 and 45° C. were tested. The process parameters are summarized in Table 7 below.

TABLE 7
Spray drying process parameters
Process parameters

| Sample | 3.3-A | 3.3-B | 3.3-C |
|---|---|---|---|
| Batch size (g solids) | 290 | 317 | 403 |
| Drying gas feed rate (acfm) | 50 | 50 | 50 |
| Liquid feed rate (g/min) | 291 | 317 | 310 |
| Atomization pressure (Psig) | 628 | 748 | 725 |
| Drying gas inlet temperature (° C.) | 109 | 114 | 142 |
| Spray dryer outlet temperature (° C.) | 36 | 35 | 45 |
| Secondary drying conditions | no secondary drying | 40° C. for 48 h | 40° C. for 48 h |
| Wet yield (g) | 211.1 | 253.3 | 360.8 |
| Wet yield (%) | 72.8 | 79.9 | 89.5 |
| Dry yield (g) | not analyzed | 193.7 | 300.3 |
| Dry yield (%) | not analyzed | 61.1 | 74.5 |

acfm . . . actual cubic feet per minute

Yield increased from the first lot sprayed (3.3-A) to the last lot sprayed (3.3-C). Yield was lower for lot A due to fixed losses, which decreased the percentage yield of smaller batch sizes relative to larger batches and increased as lots were sprayed because of carryover from previous lots and the diminishing effect of fixed losses on the later lots.

Characterization of the solid dispersions of samples 3.3-A, 3.3-B and 3.3-C was performed using X-Ray Powder Diffraction (XRPD), modulated Differential Scanning Calorimetry (mDSC) and particle size distributions (PSD). PSD was determined by laser diffraction.

XRPDs can be obtained according to the following protocol: XRPD analysis is performed using a Rigaku Miniflex 600 diffractometer. An amount of approx. 10 mg of samples 3.3-A, 3.3-B and 3.3-C is placed onto a zero-background sample disk and loaded into the auto sampler of the Rigaku Miniflex 600. Samples are analyzed using the instrument parameters as described in Table 8 below.

TABLE 8
Summary of XRPD collection parameters

| Parameter | Value |
|---|---|
| ⊖/2-⊖ | 3-40° |
| Radiation source | Cu-Kα1,2 (1.54184 Å) |
| Step size | 0.02° |
| Step rate | 2.0°/min |
| Voltage | 40 kV |
| Current | 15 mA |
| Sample disk | 0-BG |

The XRPDs obtained following the protocol from the previous paragraph with the solid dispersions of samples 3.3-A, 3.3-B and 3.3-C are displayed in FIG. 5, exhibiting a lack of sharp peaks and the presence of amorphous halos suggesting that all samples comprised amorphous compound (1).

For mDSC measurements, this protocol can be followed: an amount of 2-5 mg of samples 3.3-A, 3.3-B and 3.3-C is placed into a Tzero pan. A Tzero non-hermetic lid is affixed onto the pan and samples are analyzed in modulated mode in a scan range of 0 to 250° C. with a modulation amplitude of 1° C./min, a modulation period of 60 s and a ramp rate (heating rate) of 3° C./min. A summary of the glass transition temperatures is given in Table 9.

TABLE 9
Summary of glass transition temperatures (Tg)
of samples 3.3-A, 3.3-B and 3.3-C

| | Average |
|---|---|
| | midpoint |
| Formulation | Tg |
| 3.3-A | 116° C. |
| 3.3-B | 117° C. |
| 3.3-C | 116° C. |

The thermograms obtained following the protocol from the previous paragraph of the solid dispersion of samples 3.3-A, 3.3-B and 3.3-C all showed a single glass transition around 116° C. with no obvious melt or recrystallization events. This indicates that the solid dispersions were single-phase, comprising amorphous compound (1).

The particle size distribution (PSD) for the solid dispersions of samples 3.3-A, 3.3-B and 3.3-C can be measured by laser diffraction of dry, dispersed powder using a Sympatec HELOS laser diffraction system and RODOS dry powder feed system. The system can be run at a dispersive pressure of 3 bar with an R4 lens. The results according to this method are summarized in Table 10.

TABLE 10
Particle size distribution (PSD) of samples
3.3-A, 3.3-B and 3.3-C

| | Volume weighted particle size (μm) | | |
|---|---|---|---|
| Sample | D10 | D50 | D90 |
| 3.3-A | 11.9 | 37.0 | 79.1 |
| 3.3-B | 11.3 | 32.2 | 62.5 |
| 3.3-C | 12.7 | 36.1 | 81.1 |

### 3.4 Spray Drying Parameters

Spray drying parameters were further tested for larger batch sizes. For the tests a mixture containing 50 wt % compound (1) and 50 wt % of dispersion carrier HPMCAS-M was used. The mixture was spray dried from a spray solution composition using a solvent ratio DCM:MeOH of 90:10 (w/w) with a solids loading of 10 wt %. Solid dispersions were manufactured using an open loop custom development spray dryer SD-90, with SK 79-16 spray systems nozzle. Feed rates of 300 and 325 g/min and outlet temperatures of 44° C. and 40° C. were tested. The process parameters are summarized in Table 11 below.

TABLE 11
Spray drying process parameters
Process parameters

| Sample | 3.4-A | 3.4-B |
|---|---|---|
| Batch size (g solids) | 586.7 | 2499.3 |
| Drying gas feed rate (acfm) | 50 | 50 |
| Liquid feed rate (g/min) | 300 | 325 |
| Atomization pressure (Psig) | 938 | 693 |
| Drying gas inlet temperature (° C.) | 130 | 121 |
| Spray dryer outlet temperature (° C.) | 44 | 40 |
| Secondary drying conditions | 40° C. for 36 h | 40° C. for 39 h |
| Wet yield (g) | 414.5 | 2548 |
| Wet yield (%) | 70.7 | 102 |
| Dry yield (g) | 374.8 | 2354 |
| Dry yield (%) | 64 | 94 |

acfm . . . actual cubic feet per minute

It is assumed that the yield for sample 3.4-A was low because of the small batch size due to fixed losses. Fixed losses are independent of batch size but represent a higher portion of smaller batches. Therefore, the larger batch size of sample 3.4-B3 led to a higher yield. The characterization of both samples was performed using XRPD, mDSC and PSD. PSD was determined by laser diffraction.

XRPD scans of samples 3.4-A and 3.4-B3 were performed as described in Example 3.3 above. The diffractograms for the solid dispersion of samples 3.4-A and 3.4-B3 are displayed in FIG. 6 and showed amorphous halos with no sharp peaks, suggesting that compound (1) was in the amorphous state in both samples.

For mDSC measurements, an amount of 2-5 mg of samples 3.4-A and 3.4-B was placed into a Tzero pan and measurement was performed as described in Example 3.3 above, except that a scan range of 0 to 200° C. was used for sample 3.4-B. A summary of the glass transition temperatures is given in Table 12 below.

TABLE 12
Summary of glass transition temperatures (Tg)
of samples 3.4-A and 3.4-B

| | Average |
|---|---|
| | midpoint |
| Formulation | Tg |
| 3.4-A | 114° C. |
| 3.4-B | 111° C. |

The thermograms of the solid dispersion of samples 3.4-A and 3.4-B exhibited a single glass transition, indicating single-phase, amorphous material with no obvious peaks in the mDSC thermograms that would indicate crystalline material. The difference in glass transition temperatures compared to samples 3.3 may be attributed to different lots of compound (1) being used for the SDD manufacture and/or noise within the measurement of the instrument.

The mDSC results are consistent with the XRPD results, indicating by two orthogonal methods that the solid dispersions contained compound (1) in amorphous form.

The particle size distribution (PSD) for the solid dispersions of samples 3.4-A and 3.4-B can be measured by laser diffraction of dry, dispersed powder using a Sympatec HELOS laser diffraction system and RODOS dry powder feed system. The system can be run at a dispersive pressure of 3 bar with an R4 lens. The results according to this method are summarized in Table 13.

TABLE 13
Particle size distribution (PSD) of samples 3.4-A and 3.4-B

| | Volume weighted particle size (μm) | | |
|---|---|---|---|
| Sample | D10 | D50 | D90 |
| 3.4-A | 10.4 | 33.5 | 80.6 |
| 3.4-B | 12.6 | 40.2 | 82.3 |

### Example 4—Physical Stability of Amorphous Solid Dispersions

### 4.1 Stress Stability Study at Accelerated Stress Conditions

Physical stability of amorphous solid dispersion formulations of compound (1) equivalent to samples 3.3-A, 3.3-B and 3.3-C obtained in Example 3.3 was evaluated in an accelerated stability study. Each sample was incubated in open vials at: (i) ambient temperature/ambient humidity, (ii) ambient temperature/60% relative humidity, (iii) 40° C./ambient humidity and (iv) 40° C./75% relative humidity. Relative humidity (RH) was achieved through use of saturated salt solutions (sodium bromide for an approximate 60% RH at ambient temperature, and sodium chloride for 75% RH at 40° C.). After two and four weeks, samples were removed for analysis and characterized via XRPD to evaluate potential recrystallization.

No changes in physical properties were observed. Diffractograms of all amorphous solid dispersions remained consistent with an amorphous form of compound (1) after four weeks at all storage conditions.

### 4.2 Stress Stability Study at Harsh Stress Conditions

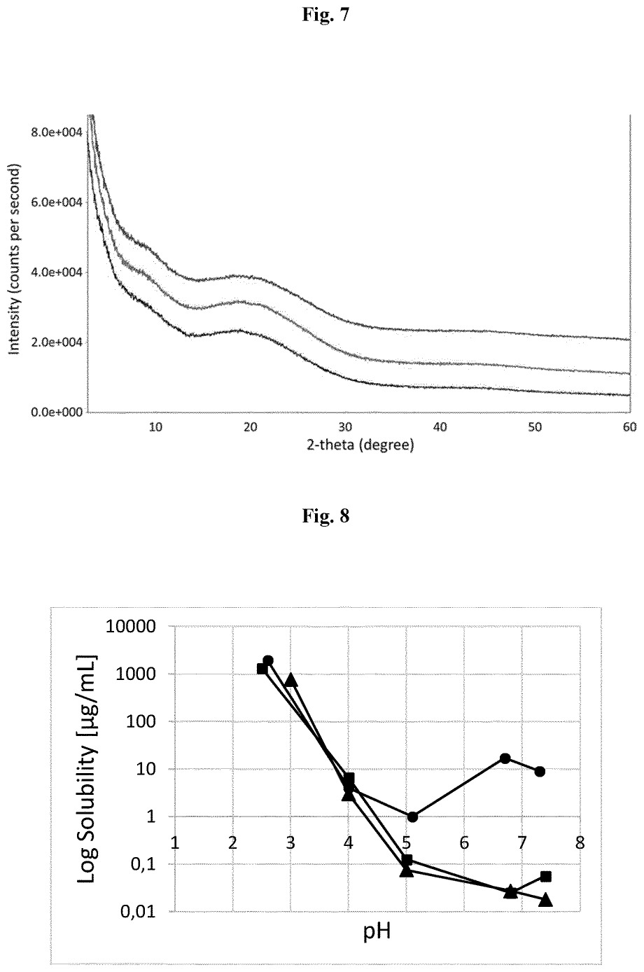

An amorphous solid dispersion (50 wt %:50 wt % compound (1):HPMCAS-M, e.g. prepared according to the procedure of Example 1) was exposed in an open container to 75° C./79% relative humidity and 80° C./76% relative humidity for three weeks. The respective XRPDs are displayed in FIG. 7. No form change was observed after exposure to these extreme stress conditions for three weeks.

### Example 5—pH-Dependent Solubility and In-Vitro Dissolution

### 5.1 Comparative Solubility Studies in Biorelevant Media and Aqueous Media at Different pH

Solubilities of an amorphous solid dispersion of compound (1) with HPMCAS-M (50 wt %:50 wt %) and of crystalline compound (1) (e.g. prepared according to Reference Example 1 and 2 hereinafter) were measured in different aqueous media at room temperature, and in biorelevant media at 37° C. The media used for solubility assays are listed in Table 14 below.

TABLE 14
Media used for solubility assays

| | Media | pH |
|---|---|---|
| Aqueous | Water milliQ ® | |
| Media | HCl 0.01N | 2 |
| | Acetate Buffer 50 mM | 4 |
| | 82 mg of anhydrous sodium acetate + 15 mL of ultrapurified water. | |
| | Adjust to pH = 4.0 with acetic acid 99.8% then make up to 20 ml with | |
| | ultrapurified water. | |
| | Acetate Buffer 50 mM | 5 |
| | 82 mg of anhydrous sodium acetate + 15 mL of ultrapurified water. | |
| | Adjust to pH = 5.0 with acetic acid 99.8% then make up to 20 ml with | |
| | ultrapurified water. | |
| | Phosphate Buffer 50 mM | 6.8 |
| | 10.2 mL of Na₂HPO₄ 0.05M solution + 9.5 mL of NaH₂PO₄ 0.05M | |
| | solution | |
| | Phosphate Buffer 50 mM | 7.4 |
| | 40.5 mL of Na₂HPO₄ 0.05M solution + 9.8 mL of NaH₂PO₄ 0.05M | |
| | solution | |
| Biorelevant | FeSSIF | 5 |
| Media | To make 5 mL of FeSSIF: Weigh 56.0 mg of SIF* Powder. Add 350 μl | |
| | of FeSSIF Buffer Concentrate then make up to 5.0 mL with | |
| | ultrapurified water. | |
| | FeSSIF can include, for example, 15 mM taurocholate, 3.75 mM | |
| | phospholipids, 319 mM sodium, 203 mM chloride and 144 mM | |
| | phosphate. | |
| | FaSSIF | 6.5 |
| | To make 5 mL of FaSSIF: Weigh 11.2 mg of SIF* Powder. Add 175 μl | |
| | of FaSSIF Buffer Concentrate then make up to 5.0 mL with | |
| | ultrapurified water. Let stand for 2 hours. | |
| | FaSSIF can include, for example, 3 mM taurocholate, 0.75 mM | |
| | phospholipids, 148 mM sodium, 106 mM chloride and 29 mM | |
| | phosphate. | |

*SIF . . . simulated intestinal fluid (3.402 g of KH₂PO₄ + 448 mg NaOH + 49 ml of ultrapurified water. Adjust to pH = 6.8 with NaOH 1N then make up to 50 ml with ultrapurified water

The following protocol has been used to prepare samples for solubility measurements:
Weigh appropriate amount of crystalline compound (1) or amorphous solid dispersion of compound (1) to reach the desired target concentration.
Addition at room temperature of selected media to reach the selected target concentration.
24 h orbital stirring at room temperature or vortex stirring at 37° C. protected from light.
Separation of the soluble fraction from the non-soluble fraction by centrifugation (15 min at 18000 rpm) followed by filtration on PTFE 0.45 μm membrane. Discard the first ~3.5 mL then collect 3 aliquots (~0.5 mL) to be analyzed.
Quantification of the 3 aliquots by UPLC-UV-MS with the appropriate calibration curve.

### UPLC-UV-MS Method:

Instrument: Waters Acquity H-Class with PDA and QDa detectors
Column: Waters Acquity BEH C₁₈, 1.7 μm, 2.1×50 mm
Flow rate: 0.65 mL/min
UV detection: 254 nm or 410 nm
Column temperature: 40° C.±2° C.
Sample temperature: 23° C.±2° C.
Injection volumes: 0.4 μL (for concentration between 1-500 μg/mL) and 9 μL (for concentration between 0.050-1 μg/mL)
Mobile phase: gradient with solutions A and B prepared as follows

| Time | A = [% ultrapurified water + | B = [% acetonitrile + |
|---|---|---|
| [min] | 0.1% formic acid] | 0.05% formic acid] |
| 0 | 90 | 10 |
| 1.8 | 5 | 95 |
| 2.6 | 5 | 95 |
| 2.7 | 90 | 10 |
| 3.9 | 90 | 10 |

Ionisation mode: ESI+/ESI−
Source temperature: 600° C.
Capillary voltage: 0.8 kV
Cone voltage: +20V/−20V

Two standard calibration curves (chromatographic UV peak area vs. concentration) are established in DMSO at 254 nm or 410 nm for each solid form and for the solid dispersion, one between 0.025 or 0.050 μg/mL and 1 μg/mL (injection volume=9 μL) and another between 1 μg/mL and 500 μg/mL (injection volume=0.4 μL). The calibration curves are linear in the whole investigated concentration range.

The results of the solubility assays obtained following the protocol from the previous paragraphs are summarized in Table 15 and displayed in FIG. 8.

TABLE 15
Equilibrium solubilities of the amorphous solid dispersion
of compound (1) with HPMCAS-M (50 wt %:50 wt %) and
crystalline forms of compound (1) in various media

| | amorphous | | compound (1) | | compound (1) | |
|---|---|---|---|---|---|---|
| | solid dispersion | | Form III | | Form IV | |
| | Final | Conc. | Final | Conc. | Final | Conc. |
| Media | pH | [μg/mL] | pH | [μg/mL] | pH | [μg/mL) |
| HCl 0.01N pH 2 | 2.6 | 1931 | 2.5 | 1323 | 3.0 | 761 |
| Acetate | 4.0 | 4 | 4.0 | 6.6 | 4.0 | 3 |
| Buffer pH 4 | | | | | | |
| Acetate | 5.1 | 1 | 5.0 | 0.126 | 5.0 | 0.075 |
| Buffer pH 5 | | | | | | |
| Phosphate | 6.7 | 17 | 6.8 | 0.025 | 6.8 | 0.027 |
| Buffer pH 6.8 | | | | | | |
| Phosphate | 7.3 | 9 | 7.4 | 0.056 | 7.4 | 0.018 |
| Buffer pH 7.4 | | | | | | |
| FaSSIF pH 6.5 | 6.3 | 70 | 6.5 | 0.803 | 6.5 | 0.726 |
| FeSSIF pH 5.0 | 4.9 | 126 | 5.0 | 49 | 5.0 | 44 |

Both crystalline forms of compound (1) have been found to be soluble in strong acidic media but solubility drops at pH ≥5. Also, in the biorelevant fasted and fed simulated intestinal fluids (FaSSIF and FeSSIF) the solubility of the crystalline forms of compound (1) is poor. It has been found that the solubility of compound (1) is significantly improved at pH ≥5 and in biorelevant media, when it is formulated as an amorphous solid dispersion with HPMCAS-M.

### 5.2 In-Vitro Dissolution of Amorphous Solid Dispersion of Compound (1) Vs Crystalline Compound (1)

The kinetic solubilities of crystalline compound (1) and of various amorphous solid dispersions formulations prepared according to the procedures disclosed in Example 1 (25 wt %:75 wt % as well as 50 wt %:50 wt % compound (1):polymer) with polymers selected from HPMCAS-M, HPMC HME 15LV, PVP-VA, Eudragit® L100 were measured in biorelevant media in the course of a pH change non-sink dissolution test.

To do so, the following protocol can be used: samples are first delivered into simulated gastric fluid (SGF) and then transferred via a dilution step into simulated intestinal fluid (SIF). The test is conducted at 3 mg/mL in 0.01N HCl as SGF (first stage) followed by 3× dilution to target 1 mg/mL in FaSSIF, pH 6.5 (+33 mM sodium phosphate for additional buffering capacity) after 30 minutes. An evaluation of “total drug” and “dissolved drug” is made. Total drug is determined by sampling the supernatant of a non-sink (saturated) sample after bench-top centrifugation (~19000 rcf, 3-5 min). Total drug includes free drug, bile salt micelles (in SIF), and colloidal species formed by drug-polymer interactions. Dissolved drug is determined by filtering the total drug supernatant through a 0.22 m filter to remove large colloidal species. Dissolved drug includes free drug and bile salt micelles.

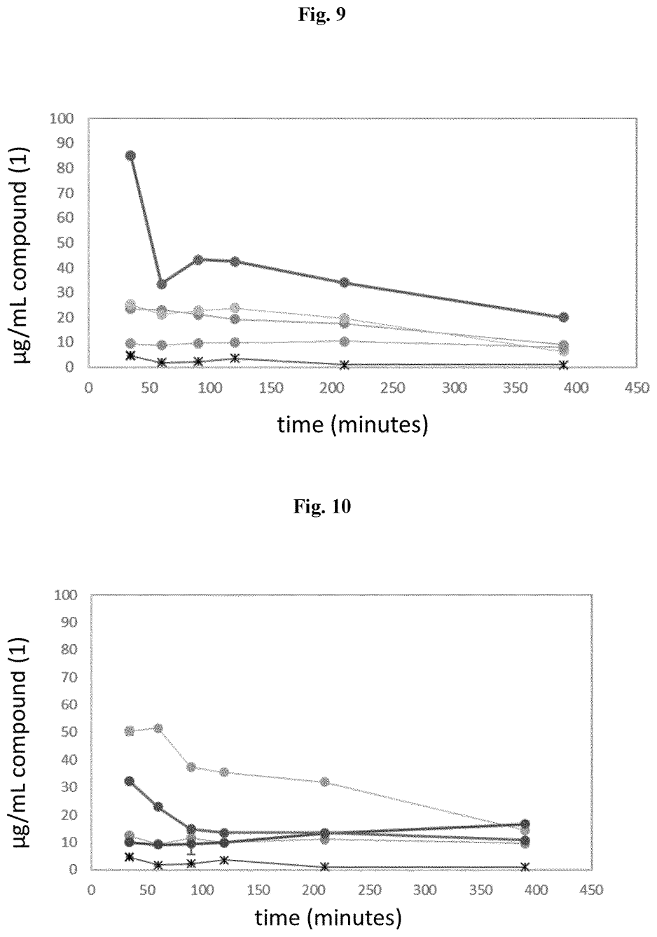

Following this protocol, it was observed that while all formulations were fully dissolved at 3 mg/mL in simulated gastric fluid (data not shown) the amorphous solid dispersion formulations demonstrated significantly more dissolved drug relative to crystalline API in simulated intestinal fluid (see FIGS. 9 and 10). It should be noted that it is typically expected that for a given polymer, as drug loading increases in an amorphous solid dispersion formulation, performance as evaluated by an increase in dissolved drug and/or colloidal species formation will either be unaffected or decrease. However, in the case of the compound (1):HPMCAS-M SDD formulations, an increase in drug loading resulted in a consistent increase in dissolved drug.

### Example 6—Pharmaceutical Compositions

### 6.1 Process to Manufacture a Tablet Comprising a Spray-Dried Solid Dispersion of Compound (1)

Film-coated tablets comprising a solid dispersion of compound (1) generally were prepared according to the following scheme unless otherwise specified.

### Step 1: Manufacture of Solid Dispersion by Spray Drying

Compound (1) and HPMCAS-MG (hydroxypropopyl methylcellulose acetate succinate-MG) are dissolved in a solvent mixture of dichloromethane (DCM) and methanol (MeOH) to produce a spray drying solution. Alternative dispersion carriers could be used instead of or in addition to HPMCAS-MG. The solution is spray dried using a suitable spray drying machine to produce a spray dried solid dispersion. This step of spray drying can be performed as described in detail in Examples 1 and 3. The spray dried solid dispersion is then further dried in a suitable dryer to remove residual solvents as described in detail in Examples 1 and 3.

Step 2: Dry Granulation of Solid Dispersion with Excipients

The dried solid dispersion is mixed with portions of microcrystalline cellulose, mannitol, croscarmellose sodium, and colloidal silicon dioxide, and the mixture of solid dispersion and fillers, disintegrant and glidant is then pre-blended and screened/delumped. Sodium stearyl fumarate as lubricant is added to the pre-blend. The intragranular blend is then granulated using a roller compactor, equipped with a 1.0 mm screen. The screened dry granules are collected for subsequent final blending.

### Step 3: Blending

The granules are blended together with a pre-screened extragranular mixture of croscarmellose sodium and colloidal silicon dioxide in a blender. Sodium stearyl fumarate is added and blended to produce a final blend.

### Step 4: Compression

The final blend is then compressed into tablet cores.

Steps 1 to 4 were followed with the ingredients given in Table 16 below.

TABLE 16
Summary of ingredients of tablet cores

| | Quantity per | Quantity per | Quantity per | |
|---|---|---|---|---|
| | tablet (mg) | tablet (mg) | tablet (mg) | |
| Ingredients | Example 6.1-A | Example 6.1-B | Example 6.1-C | Function |
| compound (1) | 15.00 | 15.00 | 60.00 | active |
| | | | | ingredient |
| hypromellose | 15.00 | 15.00 | 60.00 | dispersion |
| acetate succinate | | | | carrier |
| MG | | | | |
| microcrystalline | 36.00 | 20.00 | 80.00 | filler |
| cellulose | | | | |
| (Avicel PH-112 | | | | |
| (Dupont)) | | | | |
| mannitol | 24.00 | 42.00 | 168.00 | filler |
| (Parteck M100 | | | | |
| (Millipore) | | | | |
| croscarmellose | 7.00 | 5.00 | 20.00 | disintegrant |
| sodium | | | | |
| (Ac-Di-Sol | | | | |
| (Dupont)) | | | | |
| colloidal silicon | 1.50 | 1.50 | 6.00 | glidant |
| dioxide | | | | |
| (Millipore) | | | | |
| sodium stearyl | 1.50 | 1.50 | 6.00 | lubricant |
| fumarate | | | | |
| (JRS Pharma) | | | | |
| Total (tablet) | 100.00 | 100.00 | 400.00 | |

### 6.2 Manufacture of Film-Coated Tablets Comprising 15 mg or 60 mg of Compound (1)

Steps 1-4 can be followed by an optional film-coating step, which can be performed as outlined below.

### Step 5: Film-Coating

The film coating mixture Opadry® AMB II yellow is dispersed in water for injection using a stirrer and vessel. The tablet cores are coated with the film coating suspension in a suitable pan coater to obtain film-coated tablets comprising a solid dispersion of compound (1) and the dispersion carrier. Step 5 is optional. Alternative film coating mixtures could be used instead of Opadry® AMB II yellow.

Film-coated tablets comprising a spray-dried solid dispersion of compound (1) and HPMCAS MG (hypromellose acetate succinate where MG refers to the grade that is soluble at pH ≥6.0 and it is a granular free flowing powder) were prepared as described in Example 6.1, followed by Step 5 described above. A summary of the ingredients is given in Table 17 below.

TABLE 17
Summary of ingredients of film-coated tablets

| | Quantity per | Quantity per | Quantity per | |
|---|---|---|---|---|
| | tablet (mg) | tablet (mg) | tablet (mg) | |
| Ingredients | Example 6.2-A | Example 6.2-B | Example 6.2-C | Function |
| compound (1) | 15.00 | 15.00 | 60.00 | active |
| | | | | ingredient |
| hypromellose | 15.00 | 15.00 | 60.00 | dispersion |
| acetate succinate | | | | carrier |
| MG | | | | |
| microcrystalline | 36.00 | 20.00 | 80.00 | filler |
| cellulose | | | | |
| (Avicel PH-112 | | | | |
| (Dupont)) | | | | |
| mannitol | 24.00 | 42.00 | 168.00 | filler |
| (Parteck M100 | | | | |
| (Millipore)) | | | | |
| croscarmellose | 7.00 | 5.00 | 20.00 | disintegrant |
| sodium | | | | |
| (Ac-Di-Sol | | | | |
| (Dupont)) | | | | |
| colloidal silicon | 1.50 | 1.50 | 6.00 | glidant |
| dioxide | | | | |
| (Millipore) | | | | |
| sodium stearyl | 1.50 | 1.50 | 6.00 | lubricant |
| fumarate | | | | |
| (JRS Pharma) | | | | |
| film coating | 4.50 | 4.50 | 14.00 | coating agent |
| mixture | | | | |
| Opadry ® AMB | | | | |
| II yellow | | | | |
| Total (tablet) | 104.50 | 104.50 | 414.00 | |

The film-coated tablets comprised 15 mg or 60 mg of compound (1). Dichloromethane and methanol were used as solvents and nitrogen was used as drying gas for the solid dispersion but were removed during the process, and thus did not appear in the final product. Further, as solvent for the film coating mixture water for injection was used, but also was removed during the drying and thus not analysed.

The film-coating mixture used was Opadry® AMB II yellow 88A120087. It comprises partially hydrolysed polyvinyl alcohol as film-forming agent, talc as anti-tacking agent, sodium lauryl sulphate as lubricant and titanium dioxide, glyceryl mono and dicaprylocaprate (GMDCC) and iron oxide yellow as pigments.

### 6.3 Manufacture of Tablets Comprising 400 mg or 200 mg of Compound (1)

Non-coated tablet formulations comprising a spray-dried solid dispersion of compound (1) and HPMCAS-M in a ratio of 25:75 wt % or 50:50 wt % were prepared as described in Example 6.1 above. A summary of the ingredients is given in Tables 18 and 19 below.

TABLE 18
Summary of ingredients of tablets comprising a spray-dried solid dispersion
of 25 wt % of compound (1) and 75 wt % HPMCAS-M

| | | | Concentration | Tablet |
|---|---|---|---|---|
| | Component | Function | (wt %) | (mg/unit) |
| intra | solid dispersion of | active | 57.143 | 400.000 |
| granular | compound (1):HPMCAS-M | ingredient | | |
| (IG) | 25 wt %:75 wt % | | | |
| | microcrystalline cellulose | ductile filler | 13.929 | 97.500 |
| | (Avicel PH-101 (Dupont)) | | | |
| | mannitol | brittle filler | 13.929 | 97.500 |
| | (Parteck M100 (Millipore)) | | | |
| | croscarmellose sodium | disintegrant | 4.667 | 32.667 |
| | (Ac-Di-Sol (Dupont)) | | | |
| | colloidal silicon dioxide | glidant | 1.000 | 7.000 |
| | (EMPROVE (Millipore)) | | | |
| | sodium stearyl fumarate | lubricant | 1.000 | 7.000 |
| | (PRUV (JRS Pharma)) | | | |
| | | IG total | 91.667 | 641.667 |
| extra | microcrystalline cellulose | | 5.000 | 35.000 |
| granular | (Avicel PH-101 (Dupont)) | | | |
| | croscarmellose sodium | | 2.333 | 16.333 |
| | (Ac-Di-Sol (Dupont)) | | | |
| | colloidal silicon dioxide | | 0.500 | 3.500 |
| | (EMPROVE (Millipore)) | | | |
| | sodium stearyl fumarate | | 0.500 | 3.500 |
| | (PRUV (JRS Pharma)) | | | |
| | | total | 100.000 | 700.000 |

TABLE 19
Summary of ingredients of tablets comprising a spray-dried solid dispersion
of 50 wt % of compound (1) and 50 wt % HPMCAS-M

| | | | Concentration | Tablet |
|---|---|---|---|---|
| | Component | Function | (wt %) | (mg/unit) |
| intra | solid dispersion of | active | 35.000 | 200.000 |
| granular | compound (1):HPMCAS-M | ingredient | | |
| (IG) | 50 wt %:50 wt % | | | |
| | microcrystalline cellulose | ductile filler | 25.000 | 142.857 |
| | (Avicel PH-101 (Dupont)) | | | |
| | mannitol | brittle filler | 25.000 | 142.857 |
| | (Parteck M100 (Millipore)) | | | |
| | croscarmellose sodium | disintegrant | 4.667 | 26.667 |
| | (Ac-Di-Sol (Dupont)) | | | |
| | colloidal silicon dioxide | glidant | 1.000 | 5.714 |
| | (EMPROVE (Millipore)) | | | |
| | sodium stearyl fumarate | lubricant | 1.000 | 5.714 |
| | (PRUV (JRS Pharma)) | | | |
| | | IG total | 91.667 | 523.810 |
| extra | microcrystalline cellulose | | 5.000 | 28.571 |
| granular | (Avicel PH-101 (Dupont)) | | | |
| | croscarmellose sodium | | 2.333 | 13.333 |
| | (Ac-Di-Sol (Dupont)) | | | |
| | colloidal silicon dioxide | | 0.500 | 2.857 |
| | (EMPROVE (Millipore)) | | | |
| | sodium stearyl fumarate | | 0.500 | 2.857 |
| | (PRUV (JRS Pharma)) | | | |
| | | total | 100.000 | 571.429 |

### 6.4 Manufacture of Tablets Comprising a Solid Dispersion of Compound (1) and HPMC

It is known that solid dispersion formulations with non-enteric polymers such as HPMC are prone to gelation and, as a result, slow disintegration when formulated as tablets. Knowing this potential challenge, an initial feasibility assessment was completed for the solid dispersion comprising a spray-dried solid dispersion of 25 wt % of compound (1) and 75 wt % HPMC HME 15LV. A summary of the starting ingredients is given in Table 20 below.

TABLE 20
Summary of ingredients of tablets comprising a spray-dried solid
dispersion of 25 wt % of compound (1) and 75 wt % HPMC HME 15LV

| | | | Concentration |
|---|---|---|---|
| | Component | Function | (wt %) |
| intra | solid dispersion of | active | 62.338 |
| granular | compound (1): | | |
| (IG) | HPMC HME 15LV | ingredient | |
| | 25 wt %:75 wt % | | |
| | microcrystalline cellulose | ductile filler | 15.195 |
| | (Avicel PH-101 (Dupont)) | | |
| | mannitol | brittle filler | 15.195 |
| | Parteck M100 (Millipore)) | | |
| | croscarmellose sodium | disintegrant | 5.091 |
| | (Ac-Di-Sol (Dupont)) | | |
| | colloidal silicon dioxide | glidant | 1.091 |
| | EMPROVE (Millipore)) | | |
| | sodium stearyl fumarate | lubricant | 1.091 |
| | PRUV (JRS Pharma)) | | |
| | | IG total | 100.00 |

The formulation described in Table 20 did not disintegrate as expected. A formulation approach that improved disintegration was a 50% dilution of the intragranular blend of Table 20 with increased amount of microcrystalline cellulose and mannitol (24 wt % each) and only 2 wt % croscarmellose sodium and a tablet architecture leveraging granulated and extragranular components. The final formulation contained 50 mg compound (1) in a 700 mg tablet.

### 6.5 Characterization of Tablet Cores and Film Coated Tablets by X-Ray Powder Diffraction (XRPD)

Tablet cores (Example 6.1-C) and film-coated tablets (Example 6.2-C) were investigated by XRPD in order to confirm absence of crystalline compound (1) such as Form III and Form IV. To do so, the following protocol can be followed: samples are prepared by slightly grinding the tablet cores or film coated tablets in a mortar with a pestle followed by homogenously mixing the obtained powder with a spatula. The obtained powder is then measured by XRPD using an X'pert PRO diffractometer and applying the following settings and measurement parameters:

TABLE 21
experimental parameters for XRPD measurements
Settings

| | |
|---|---|
| Configuration | Reflection Transmission Spinner |
| Generator | Cu 45 kV/40 mA |
| Focus | Line Focus |
| Geometry | Bragg-Brentano |
| Radiation | Cu Kα (λ = 1.54184 Å) |
| Sample Stage | Spinning |
| Spinner revolution | 1 rps |
| Beam Knife | Beam Knife for linear detectors |
| | at lower position |

Measurement parameters

| | |
|---|---|
| Scan axis | Gonio |
| Scan Mode | Continuous |
| Scan Range | 4.0-40° 2-theta |
| Step size | 0.0167º |
| Time per step | 10 s |

XRPDs of the tablet core of Example 6.1-C and the film-coated tablet of Example 6.2-C obtained following the procedure from the previous paragraph are displayed in FIGS. 15 and 16. Both formulations contain crystalline excipients that are combined with an amorphous solid dispersion containing compound (1). The diffraction peaks present in the XRPDs are attributed to those excipients. The absence of Form IV is indicated by the absence of a peak at e.g. (5.8±0.2)°, and the absence of Form III is indicated by the absence of a peak at e.g. (6.2±0.2)°.

### Example 7—Determination of Properties of Tablets Containing a Spray-Dried Solid Dispersion of Compound (1)

### 7.1 In Vitro Dissolution Profile in Phosphate Buffer pH 2.0

A dissolution test comparison was performed comparing conventional film-coated tablets comprising a total of 15 mg of the crystalline compound (1) and the film-coated tablet comprising 15 mg of compound (1) as a spray-dried solid dispersion with HPMCAS MG of Example 6.2-B.

The comparative film-coated tablets with crystalline compound (1) contained 5 mg of compound (1), 64.5 mg silicified microcrystalline cellulose comprised of colloidal silicon dioxide and microcrystalline cellulose as filler, 21 mg anhydrous lactose as filler, 3 mg type A sodium starch glycolate as disintegrant, 5 mg hydroxypropyl cellulose as binder, 0.5 mg colloidal silicon dioxide as glidant, 1 mg magnesium stearate of vegetable origin as lubricant, 4.5 mg of film-coating mixture (e.g. Opadry® yellow 03B120053). For the testing three 5 mg tablets were used.

To perform a dissolution test comparison, the following protocol can be used: dissolution testing is performed in 20 mM phosphate buffer (NaH₂PO₄) at a pH of 2.0, using an Agilent 708-DS Apparatus with 850-DS sampling station at 37° C. The 15 mg tablet comprising compound (1) as a solid dispersion equivalent to sample 6.2-B and three 5 mg tablets adding up to a total of 15 mg of compound (1) in crystalline form are suspended in the buffer solution. Dissolution profiles are evaluated under the following conditions: shaft rotation 50 rpm, medium volume 900 mL, sample volume 3 mL. The amount of compound (1) in the buffer is measured by HPLC at regular intervals over 60 minutes. The % dissolution is calculated by following equation (A):

$$\% Dissolved = ( ( {A}_{smp} \times {C}_{S 1} \times {DF}_{smp} ) / ( {A}_{S 1} \times LC ) ) \times 100 \qquad ( A )$$

where:
Aₛₘₚ is the sample peak area
C_{S1} is the Standard 1 concentration, 0.017 mg/mL compound (1)
DFₛₘₚ is the sample dilution factor, 900 mL
A_{S1} is the average peak area response from the first five Standard 1 injections
LC is the Tablet label claim; 15 mg.

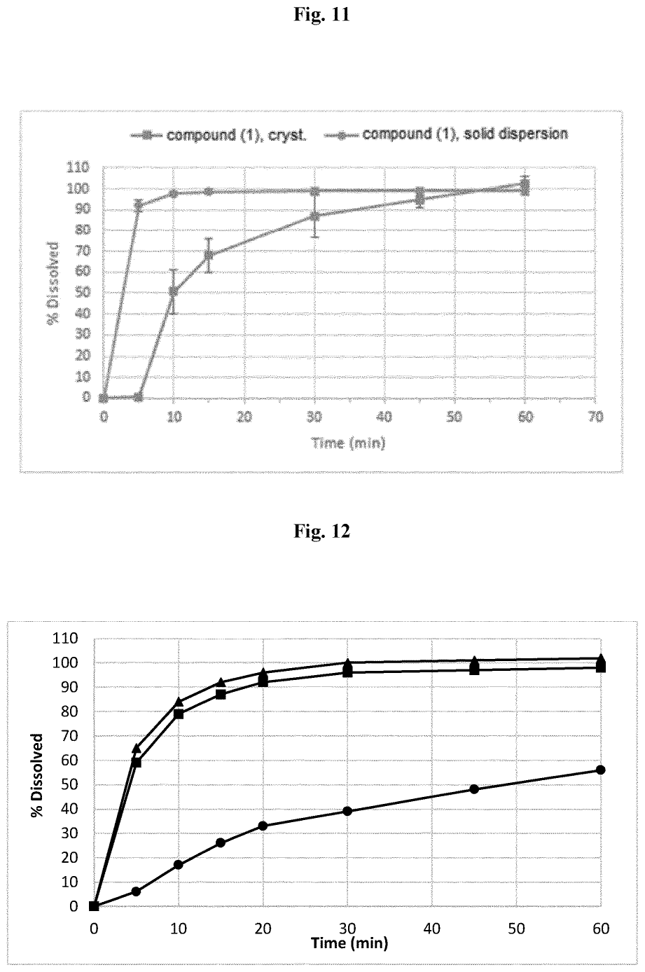

The results of the in vitro dissolution comparison between the conventional tablet and the solid dispersion tablet in pH 2.0 buffer, obtained according to the protocol described in the previous paragraph, are shown in FIG. 11. As can be seen, the tablet comprising the solid dispersion showed faster initial drug release than the tablet comprising crystalline compound (1).

7.2 In Vitro Dissolution Profile in Phosphate Buffer pH 6.8 with 0.1% SDS

A dissolution test comparison was performed comparing conventional film-coated tablets comprising a total of 60 mg (3×20 mg) of the crystalline compound (1), the film-coated tablet comprising a total of 60 mg (4×15 mg) of compound (1) as a spray-dried solid dispersion with HPMCAS MG of Example 6.2-A and the film-coated tablet comprising 60 mg of compound (1) as a spray-dried solid dispersion with HPMCAS MG of Example 6.2-C.

The conventional film-coated tablets with crystalline compound (1) contained 20 mg of compound (1), 49.5 mg silicified microcrystalline cellulose comprised of colloidal silicon dioxide and microcrystalline cellulose as filler, 21 mg anhydrous lactose as filler, 3 mg type A sodium starch glycolate as disintegrant, 5 mg hydroxypropyl cellulose as binder, 0.5 mg colloidal silicon dioxide as glidant, 1 mg magnesium stearate of vegetable origin as lubricant, 4.5 mg of film-coating mixture (e.g. Opadry® yellow 03B120053). For the testing three 20 mg tablets were used.

Dissolution testing was performed under the conditions outlined in Table 22.

TABLE 22
In vitro dissolution conditions

| | |
|---|---|
| Apparatus: | USP II, Paddle |
| Medium: | pH 6.8 buffer with 0.1% SDS |
| Speed of Rotation: | 50 rpm (Start to 45 min), |
| | 250 rpm (45 to 60 min) |
| Volume: | 900 mL |
| Temperature: | 37° C. |
| Number of Replicate Runs | 12 |
| Sampling Time Points | 5, 10, 15, 20, 30, 45, 60 (infinity) |

The % dissolution (same as % dissolved) was calculated as described above in Example 7.1. The results of the in vitro dissolution comparison between the conventional tablet and the solid dispersion tablets in pH 6.8 buffer are shown in FIG. 12 As can be seen, the tablets comprising the solid dispersion have similar dissolution profiles and show faster initial drug release than the tablet comprising crystalline compound (1). In addition, tablets comprising the solid dispersion dissolve completely, in contrast to tablets containing crystalline compound (1).

### 7.3 In Vitro Determination of Bioaccessibility

Bioavailability in humans was evaluated using the dynamic in vitro gastrointestinal model for the simulation of the physiological processes occurring in human stomach and small intestine tiny-TIM.

A conventional tablet of crystalline compound (1) (conventional formulation) and a tablet comprising a solid dispersion of compound (1) (SDD formulation) were tested in the tiny-TIM model.

The conventional formulation included 100 mg of compound (1), 247.5 mg of silicified microcrystalline cellulose and 105 mg of anhydrous lactose as fillers, 25 mg of hydroxypropyl cellulose as a binder, 15 mg of sodium starch glycolate as a disintegrant, 2.5 mg of colloidal silicon dioxide as a glidant, and 5 mg of magnesium stearate as a lubricant.

The SDD formulation tested in the tiny-TIM model corresponds to Example 6.2-A, as shown in Table 17.

### Tiny-TIM Study Protocol:

### Meal Matrices in Tiny-TIM Set Up

For simulation of the fasted state condition, a glass of water (240 mL) is administered to the tiny-TIM system.

### The Tiny-TIM Test System:

The study is performed in the TNO dynamic, multi-compartmental in vitro system of the stomach and small intestine (tiny-TIM).

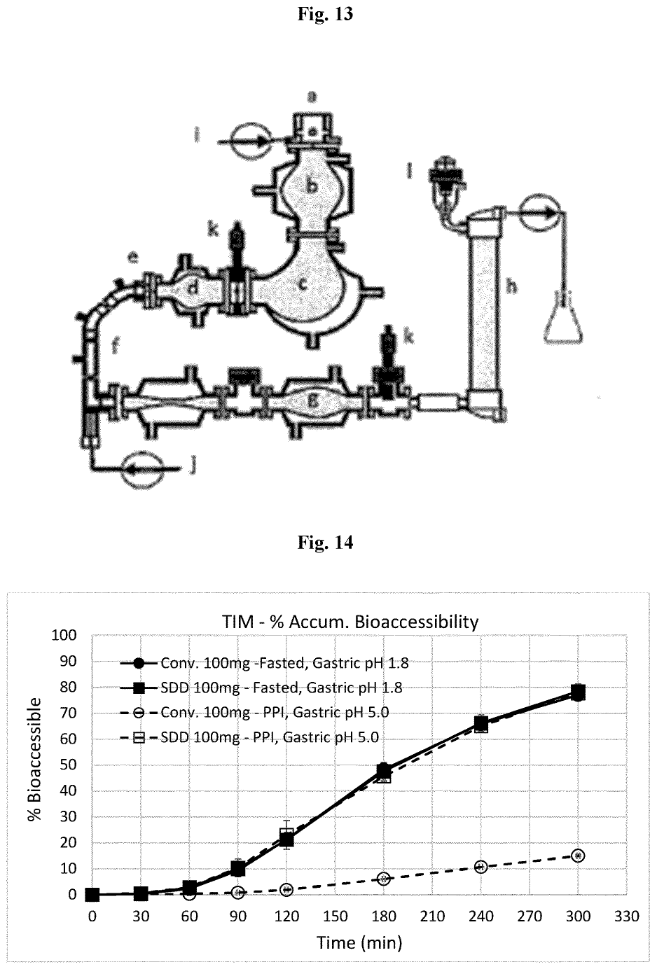

The tiny-TIM system consists of a gastric compartment and one small intestinal compartment (FIG. 13). This compartment is composed of two glass units with a flexible silicone inner wall enclosing the luminal material. The space between the inner and outer walls is filled with water. Peristaltic mixing of the chyme is the result of alternate compression and relaxation of the flexible inner wall. The compartments are connected by peristaltic valve pumps that successively open and close, allowing the chyme to transit over time through the compartments. This way, oral dosage forms/API's are exposed to locally changing and physiological relevant conditions in the stomach and of the small intestine for tiny-TIM.

The tiny-TIM system mimics the intraluminal pH, enzyme activity, bile salt concentrations, peristaltic movements, and gastrointestinal transit of the contents. The set-points for gastrointestinal simulation are controlled and monitored by specific computer programs. Released and dissolved drug molecules are removed from the intestinal lumen by semipermeable membrane units connected to the small intestinal compartment. This allows the assessment of the so-called bioaccessible fraction, i.e. the fraction of the drug which is available for small intestinal absorption.

### Simulated Gastro-Intestinal Conditions:

The experiments in tiny-TIM are performed under simulation of the average physiological conditions in the gastrointestinal tract as described for humans in the fasted state. These conditions include especially the dynamics of gastric emptying and pH decline, intestinal transit times, housekeeper wave, the gastric and the intestinal pH values (Tables 23 and 24), and the composition and activity of the secretion products. The digested and soluble (low-molecular) compounds are removed continuously from the intestinal compartment via a special membrane system.

Prior to the performance of each experiment the secretion fluids (e.g. gastric juice with enzymes, electrolytes, bile, and pancreatic juice) are freshly prepared, the pH electrodes calibrated, and semipermeable membrane (hollow fiber) units installed.

TABLE 23
Parameters simulated in the tiny-TIM, describing the
average gastrointestinal physiological conditions
of healthy young adults for fasted state

| Tiny-TIM | Fasted state |
|---|---|
| Gastric compartment tiny-TIM | |
| Intake (total) | 270 g |
| Water and artificial saliva | 240 g |
| Gastric start fluid | 30 g |
| Gastric emptying T1/2 | 20 min |
| House keeper wave | 60 min |
| Gastric pH | 3.0 to 1.8 in 30 min |
| Small intestinal compartment tiny-TIM | |
| pH intestinal compartment | 6.5 |
| Experimental duration | 5 hours |

TABLE 24
Parameters simulated in the tiny-TIM, describing
the average gastrointestinal physiological conditions
of healthy young adults for fasted state plus PPI

| Tiny-TIM | Fasted state + PPI 1 |
|---|---|
| Gastric compartment tiny-TIM | |
| Intake (total) | 270 g |
| Meal (HFM) | — |
| Water and artificial saliva | 240 g |
| Gastric start fluid | 30 g |
| Gastric emptying T1/2 | 20 min |
| House keeper wave | 60 min |
| Gastric pH | 5.0 continuous |
| Small intestinal compartment tiny-TIM | |
| pH intestinal compartment | 6.5 |
| Experimental duration | 5 hours |

### Housekeeper Wave

The housekeeper wave (HKW) is simulated after 60 minutes by automated transfer of residual material from the gastric compartment to the intestinal compartment.

### Experiments

The experiments are performed as duplicate experiments. All runs are performed under yellow light to prevent degradation of compound (1).

### Sampling

### Filtrate

Filtration of released and dissolved/solubilized drug molecules from the intestinal lumen via a semi-permeable membrane unit (Fresenius plasmaFlux® P1dry) allows the assessment of the so-called bio-accessible fraction, i.e. the fraction of the drug which is available for small intestinal absorption. Filtrate is collected in the following time intervals: 0-30, 30-60, 60-90, 90-120, 120-180, 180-240 and 240-300 minutes from the start of the experiments (FIG. 13, sampling spot H). Analyses of these samples generates data on the bioaccessibility and the availability for absorption of compound (1). The collected volume per time period is measured and sub-samples taken, instantly diluted in organic solvent and stored at 2-10° C., protected from light, until analysis.

### Residues

At the end of each experiment the residues in the gastric compartment and in the small intestinal compartment plus filter unit are collected, measured, and analyzed. These residue samples represent the non-bioaccessible fraction. This rinse is pooled with the residue sample of the same compartment, the volume measured and stored at 2-10° C., protected from light, until analysis.

### Storage of Back-Up Samples

The back-up samples are stored at ≤−18° C., protected from light, for 1 month after finalization of the study report, thereafter the samples are destroyed.

### Sample Analysis

The collected samples are analyzed for the concentration of compound (1).

### Calculation of Results

The absolute amount of the API in a sample is calculated by multiplying the analyzed concentration in the sample with the collected volume (equation 1).

$$A ( mg ) = {C}_{sample} ( μ g / mL ) \cdot {10}^{- 3} \cdot {V}_{sample} [ mL ] \qquad ( 1 )$$

The recovery of the API is determined by the sum of all amounts recovered in the filtrate fractions of the intestinal compartment and in the residue and rinse fractions of the gastric and intestinal compartments and the drug product. The total recovery is expressed as % of amount added (equation 2).

$$Recovery ( \% ) = \frac{\sum {A}_{filtrate} ( mg ) + \sum {A}_{residues} ( mg )}{{A}_{added} ( mg )} \cdot 100 \% \qquad ( 2 )$$

The bioaccessibility (% of intake) is calculated by expressing the amount of API recovered from the filtrate as a percentage of the intake (equation 3).

$$Bioaccessibility ( \% of dose ) = \frac{\sum {A}_{filtrate} ( mg )}{{A}_{added} ( mg )} \cdot 100 \% \qquad ( 3 )$$

The results of the duplicate runs are presented as mean SD. For the SD, in Microsoft® Excel® the STDEVP function is used (equation 4).

$$Stdevp = \sqrt{\frac{\sum {( x - \overline{x} )}^{2}}{n}} \qquad ( 4 )$$

### Statistics

No statistical analysis is performed for this study.

### Results

The bioaccessibility profiles obtained from the tiny-TIM protocol above are presented in FIG. 14 for both conventional (conv.) formulation and the SDD formulation under two conditions, fasted state and under simulated PPI conditions, i.e. higher gastric pH. Under fasted conditions with low gastric pH (3.0 to 1.8 in 30 min), comparable bioaccessibility between conv. and SDD tablets was observed. Under PPI conditions (fasted with gastric pH 5), the conventional tablet showed about 5-fold reduction in bioaccessibility, while the SDD tablet was not affected. Therefore, in contrast to the conventional tablet, the performance of the SDD formulation is pH-independent. The tablets of Example 6.2-B and 6.2-C described in Table 17 have been tested under the same tiny-TIM study protocol and showed comparable results.

### 7.4 In Vivo Relative Bioavailability Clinical Study

A clinical study was performed to assess the relative bioavailability of compound (1) in two different oral formulations, namely as conventional tablet comprising the crystalline form of compound (1) and as a tablet comprising a solid dispersion of compound (1) according to the invention. In addition, the effects of food and multiple-doses of the protein pump inhibitor (PPI) rabeprazole on the pharmacokinetics of single-dose administration of compound (1) were investigated following oral administration of the above-mentioned solid dispersion formulation in healthy male subjects.

### 7.4.1 Protocol

A number of 16 healthy male subjects, aged 18 to 45 years (inclusive) and with a body mass index (BMI) of 18.5 to 29.9 kg/m² (inclusive) is included in the study. The study design is an open-label, randomized, four-way crossover trial. The primary endpoints are the area under the plasma concentration-time curve from time 0 (t0) corresponding to the timepoint of drug administration until time z (tz) corresponding to the last quantifiable timepoint (AUC_{0-tz}) and the maximum concentration in plasma (Cₘₐₓ) of compound (1). The secondary endpoint is the area under the plasma concentration-time curve from t0 extrapolated to infinity (AUC_{0-∞}) of compound (1).

Thus, the objectives of the trial are to investigate
Test 1: the relative bioavailability under fasting conditions of two different tablet formulations of compound (1) in crystalline form and as a solid dispersion,
Test 2: the relative bioavailability of compound (1) formulated as a solid dispersion under fasting and fed conditions, and
Test 3: the relative bioavailability of compound (1) formulated as a solid dispersion given alone and together with rabeprazole under fasting conditions.
Test product 1: The comparative film-coated tablets with crystalline compound (1) contain 5 mg or 20 mg of compound (1) and 64.5 or 49.5 mg, respectively, silicified microcrystalline cellulose comprised of colloidal silicon dioxide and microcrystalline cellulose as filler, 21 mg anhydrous lactose as filler, 3 mg type A sodium starch glycolate as disintegrant, 5 mg hydroxypropyl cellulose as binder, 0.5 mg colloidal silicon dioxide as glidant, 1 mg magnesium stearate of vegetable origin as lubricant, 4.5 mg of film-coating mixture (e.g. Opadry® yellow 03B120053).
Test product 2: Film-coated tablets comprising 15 mg of compound (1) as a spray-dried solid dispersion with HPMCAS MG as defined in Example 6.2-A.
Test product 3: Proton pump inhibitor rabeprazole gastroresistant tablets PARIET® of a strength of 20 mg.

The reference treatment (R or TF1) consists of a total dose of 30 mg crystalline compound (1) in form of test product 1 (1 tablet à 20 mg and 2 tablets à 5 mg) administered orally with 240 Ml of water after an overnight fast of at least 10 h on day 1.
Test treatment 1 (T1 or NF1) consists of a total dose of 30 mg compound (1) in form of a solid dispersion as test product 2 (2 tablets à 15 mg) administered orally with 240 mL of water after an overnight fast of at least 10 h on day 1.
Test treatment 2 (T2) consists of a total dose of 30 mg compound (1) in form of a solid dispersion as test product 2 (2 tablets à 15 mg) on day 1 administered under fed conditions after a high-fat, high-calorie breakfast. The total caloric content of the high fat, high-calorie breakfast is supplied approximately as follows: 150 kcal as protein, 250 kcal as carbohydrate, and 500 to 600 kcal as fat; Ingredients: 2 chicken eggs (whole content) for scrambled eggs 192 kcal; 10 g butter for frying scrambled eggs 75 kcal, 35 g fried bacon 186 kcal, 2 toasted slices of wheat bread 130 kcal, 15 g butter for buttering toast slices 113 kcal, 115 g hash brown potatoes 132 kcal, 240 mL whole milk (3.5% fat) 156 kcal; Sum 984 kcal.
Test treatment 3 (T3) consists of a total dose of 30 mg compound (1) in form of a solid dispersion as test product 2 (2 tablets à 15 mg) administered under fasting conditions. Subjects in T3 further receive test product 3 in a total dose of 200 mg of rabeprazole in daily doses of 40 mg once daily (2 tablets à 20 mg) on four days prior to and on the day of administration of compound (1).

Blood sampling is performed up to 118 h post administration of compound (1) for all treatments to analyze for plasma concentrations of compound (1). Plasma concentration time profiles are evaluated by non-compartmental analysis to calculate respective PK parameters. Relative bioavailability is estimated by the ratios of the geometric means (T1/R, T2/T1 and T3/T1) for the primary and secondary endpoints. Additionally, their two-sided 90% confidence intervals (CIs) are provided. This method corresponds to the two one-sided t-test procedure, each at a 5% significance level. Since the main focus is on estimation and not on testing, a formal hypothesis test and associated acceptance range is not specified. The statistical model was analysis of variance (ANOVA) on the logarithmic scale including effects for sequence, subjects nested within sequences, period and treatment. CIs are calculated based on the residual error from the ANOVA. Descriptive statistics were calculated for all endpoints. Pharmacokinetic analyses are performed on the Pharmacokinetic parameter analysis set (PKS) and safety analyses are performed on the Treated set (TS). No formal interim analysis is planned or performed.

### 7.4.2 Results

From the 16 subjects planned to be included in the trial, 13 subjects completed the study. For the treatment comparisons, 12 subjects were evaluable for the relative bioavailability comparison of T1 to R, 9 subjects for the food effect evaluation (comparison of T2 to T1) and 11 subjects for the evaluation of the drug-drug interaction between compound (1) and rabeprazole (comparison of T3 to T1). The relative bioavailability comparison showed a decreased variability for the tablets comprising a solid dispersion of compound (1) (T1) compared to the tablets comprising crystalline compound (1) (R). The exposure of T1 was in average increased by 3% (Cₘₐₓ) and 35% (AUC0-tz) in comparison to R. The food effect evaluation showed that exposure was decreased under fed conditions (T2) in comparison to fasted (T1), in average by −46% for Cmax and −26% for AUC0-tz. Pretreatment with rabeprazole did not relevantly change the exposure of the tablets comprising a solid dispersion of compound (1) (in average −13% for Cmax and −3% for AUC0-tz) suggesting that there is no relevant DDI between compound (1) and proton pump inhibitors or other pH-increasing comedications.

Results of the trial are discussed in more detail below.

Trial Subjects and Compliance with the Clinical Trial Protocol

A total of 13 subjects received trial medication and completed the planned observation time. No important protocol violations were reported. Of the 13 healthy male subjects treated in the trial, 12 subjects (92.3%) were White, 1 subject (7.7%) was Black or African American. The mean age of the subjects was 34.8 years (standard deviation [SD]=5.8 years); age ranged from 25 to 45 years. The mean BMI was 25.49 kg/m² (SD=3.03 kg/m²); BMI ranged from 20.7 to 29.5 kg/m². The treatment groups were similar with respect to demographic and baseline characteristics.

Twelve subjects received the reference treatment (R), twelve subjects received the test treatment 1 (T1), nine subjects received the test treatment 2 (T2) and eleven subjects received the test treatment 3 (T3) in a randomized way and separated by a washout interval of at least 14 days between the administrations of compound (1) and subsequent treatments.

Relative bioavailability of the formulations NF (T1) and TF1 (R) under fasted conditions is given in Table 25. The adjusted geometric mean ratios for the primary and secondary endpoints in subjects on treatment T1/R ranged between 129.1% and 139.3%, with 90% CIs ranging between 87.7% and 221.3% (Table 25). The variability of the pharmacokinetic (PK) parameters Cₘₐₓ (geometric coefficient of variation [gCV]93.1%), AUC0-tz (gCV 52.7%) and AUC_{0-∞} (gCV 52.7%) was higher for subjects on treatment R compared with subjects on treatment T1 with Cₘₐₓ (gCV 37.3%), AUC_{0-tz}(gCV 18.8%) and AUC_{0-∞} (gCV 19.2%). The trend towards increased bioavailability of the NF was not consistently observed in all subjects, however an overall trend towards increased oral bioavailability has been shown.

TABLE 25
Adjusted geometric means and relative bioavailability of compound (1) NF fasted
(T1) vs TF1 fasted (R) with subjects as random effect - Pharmacokinetic set

| | TF1 fasted (R) | | NF fasted (T1) | | Ratio | | | Intra- |
|---|---|---|---|---|---|---|---|---|
| PK | | adj. | | adj. | T1/R | | 90% CI | ind. gCV |
| parameter | N | Mean | N | gMean | (%) | gSE | (%) | (%) |

Primary endpoints

| | | | | | | | | | |
|---|---|---|---|---|---|---|---|---|---|
| AUC_{0-tz} | 12 | 4146 | 12 | 5608 | 135.2 | 1.179 | 99.7 | 183.6. | 39.7 |
| [nmol · h/L] | | | | | | | | | |
| Cₘₐₓ | 12 | 288 | 12 | 401 | 139.3 | 1.283 | 87.7 | 221.3 | 62.3 |
| [nmol/L] | | | | | | | | | |

Secondary endpoint

| | | | | | | | | | |
|---|---|---|---|---|---|---|---|---|---|
| AUC_{0-∞} | 11 | 4580 | 12 | 5915 | 129.1 | 1.172 | 96.2 | 173.4 | 36.6 |
| [nmol · h/L] | | | | | | | | | |

Abbreviations:
adj = adjusted,
gCV = geometric coefficient of variation,
gMean = geometric mean,
gSE = geometric standard error,
ind = individual

ANOVA results comparing the primary and secondary endpoints of the NF formulation when given fasted (T1) or after intake of a high-fat high-calorie meal (T2) is provided in Table 26. The adjusted gMean ratios for the primary and secondary endpoints in subjects on treatment T2/T1 ranged between 53.5% and 74.9%, with 90% CIs ranging between 40.5% and 81.7% (Table 26). The values for Cₘₐₓ, AUC_{0-tz}, and AUC_{0-∞} were lower in subjects receiving treatment under fed condition, indicating a negative food effect.

TABLE 26
Adjusted geometric means and relative bioavailability of compound (1) NF fed
(T2) vs NF fasted (T1) with subject as random effect - Pharmacokinetic set

| | NF fasted (T1) | | NF fed (T2) | | Ratio | | | Intra- |
|---|---|---|---|---|---|---|---|---|
| PK | | adj. | | adj. | T2/T1 | | 90% CI | ind. gCV |
| parameter | N | gMean | N | gMean | (%) | gSE | (%) | (%) |

Primary endpoints

| | | | | | | | | | |
|---|---|---|---|---|---|---|---|---|---|
| AUC_{0-tz} | 12 | 5628 | 9 | 4178 | 74.2 | 1.050 | 67.6 | 81.6 | 9.8 |
| [nmol · h/L] | | | | | | | | | |
| Cₘₐₓ | 12 | 398 | 9 | 213 | 53.5 | 1.162 | 40.5 | 70.8 | 31.0 |
| [nmol/L] | | | | | | | | | |

Secondary endpoint

| | | | | | | | | | |
|---|---|---|---|---|---|---|---|---|---|
| AUC_{0-∞} | 12 | 5921 | 9 | 4434 | 74.9 | 1.046 | 68.6 | 81.7 | 9.0 |
| [nmol · h/L] | | | | | | | | | |

Abbreviations:
adj = adjusted,
gCV = geometric coefficient of variation,
gMean = geometric mean,
gSE == geometric standard error,
ind = individual

ANOVA results comparing the primary and secondary endpoints of the NF formulation when given fasted in the absence (T1) and presence of coadministration with the proton pump inhibitor (PPI) rabeprazole (T3) is presented in Table 27. The adjusted geometric mean (gMean) ratios of the endpoints in subjects on treatment T3/T1 ranged between 87.0% and 97.1%, with 90% CIs ranging between 66.8% and 113.2% (Table 27). The PK parameters and profiles for T1 and T3 were similar while the time from (last) dosing to the maximum measured concentration of the analyte in plasma (tₘₐₓ) appeared to be delayed in the presence of rabeprazole. Taken together, the results suggest that rabeprazole does not interfere with the PK of compound (1).

TABLE 27
Adjusted geometric means and relative bioavailability of compound (1) NF fasted +
rabeprazole (T3) vs NF fasted (T1) with subject as random effect - Pharmacokinetic set

| | | | NF fasted + | | | | | |
|---|---|---|---|---|---|---|---|---|
| | NF fasted (T1) | | Rabeprazole (T3) | | Ratio | | | Intra- |
| PK | | Adj. | | Adj. | T3/T1 | | 90% CI | ind. gCV |
| parameter | N | gMean | N | gMean | (%) | gSE | (%) | (%) |

Primary endpoints

| | | | | | | | | | |
|---|---|---|---|---|---|---|---|---|---|
| AUC_{0-tz} | 12 | 5591 | 11 | 5432 | 97.1 | 1.069 | 85.8 | 110.0 | 13.8 |
| [nmol · h/L] | | | | | | | | | |
| Cₘₐₓ | 12 | 393 | 11 | 341 | 87.0 | 1.154 | 66.8 | 113.2 | 30.5 |
| [nmol/L] | | | | | | | | | |

Secondary endpoint

| | | | | | | | | | |
|---|---|---|---|---|---|---|---|---|---|
| AUC_{0-∞} | 12 | 5883 | 11 | 5703 | 96.9 | 1.071 | 85.3 | 110.2 | 14.2 |
| [nmol · h/L] | | | | | | | | | |

Abbreviations:
adj = adjusted,
gCV = geometric coefficient of variation,
gMean = geometric mean,
gSE = geometric standard error,
ind = individual

### Example 8—an Open Label, Phase I Dose Escalation Trial, with Dose Confirmation and Expansion, of Compound (1) as Monotherapy in Patients with Advanced or Metastatic Solid Tumors with HER2 Aberrations

### 8.1 Protocol:

This is a First-in-Human dose escalation and expansion trial to determine the Maximum Tolerated Dose (MTD) and explore safety, pharmacokinetics, pharmacodynamics and first signs of efficacy of compound (1) as monotherapy in patients with HER2 aberration-positive advanced and/or metastatic solid tumours.

The dose escalation part of the trial (also referred to as Phase Ia) includes consecutive cohorts of patients treated with escalating doses of compound (1).

### 8.1.1 Objectives:

The objectives of the dose escalation part of the trial are to:
Investigate the safety, tolerability, and pharmacokinetics (PK) of escalating doses of compound (1) as monotherapy administered orally bis in die (BID, twice daily dosing) or quaque die (QD, once a day) in patients with advanced and/or metastatic solid tumours harbouring HER2 aberrations;
Determine the MTD and/or the Recommended Phase II Dose (RP2D) of compound (1) monotherapy administered orally for each studied regimen.

### 8.1.2 Endpoints:

The primary endpoints of the dose escalation part of the trial are:
MTD, defined as the highest dose with less than 25% risk of the true Dose Limiting Toxicity (DLT) rate being equal to or above 33% during the MTD evaluation period in any studied regimen;
Number of patients with DLTs in the MTD evaluation period.

The MTD evaluation period is defined as the first 21 days of treatment (first cycle).

The secondary endpoints of the dose escalation part of the trial are:
Number of patients experiencing DLTs during the entire treatment period;
The following PK parameters of compound (1) after first and multiple dose administration of the compound on Day 1 and 15 (if feasible):
   Cₘₐₓ: maximum measured concentration of compound (1) in plasma;
   AUC₀₋₁₂: area under the concentration-time curve of compound (1) in plasma.

If assessable and applicable, the following further endpoints are evaluated:
Number of patients experiencing adverse events (AEs) during the on-treatment period
Objective response (OR), defined as best overall response of complete response (CR) or partial response (PR), where best overall response is determined according to RECIST version 1.1 as assessed by the investigator, from the first treatment administration until the earliest of disease progression, death or last evaluable tumor assessment before start of subsequent anti-cancer therapy, loss to follow-up or withdrawal of consent.
Disease control (DC), defined as best overall response of complete response (CR) or partial response (PR) or stable disease (SD) where best overall response is defined according to RECIST version 1.1 as assessed by the investigator, from until the earliest of disease progression, death or last evaluable tumor assessment before start of subsequent anti-cancer therapy, loss to follow-up or withdrawal of consent.
Duration of objective response (DoR), defined as the time from first documented complete response (CR) or partial response (PR) until the earliest of disease progression or death among patients with objective response.
Duration of disease control (DoDC), defined as the time from first treatment administration until the earliest of disease progression or death, among patients with disease control.
If data allows, the PK parameters to be calculated for compound (1) as monotherapy include:
   AUC_{0-∞}: area under the concentration-time curve in plasma over the time interval from 0 extrapolated to infinity.
AUC_{0-tz}: area under the plasma concentration-time curve over the time interval from 0 to the last measured time point (tz).
Cₘᵢₙ: minimum measured plasma concentration in plasma.
t_{1/2}: terminal half-life of the analyte in plasma.
tₘₐₓ: time from dosing to Cₘₐₓ in plasma.

### 8.1.3 Dose Escalation

The dose escalation is performed according to an open-label design. The data obtained from the trial determines the MTD estimate based on a Bayesian logistic regression model (BLRM) with overdose control (Neuenschwander B, Branson M, Gsponer T. Critical aspects of the Bayesian approach to phase I cancer trials. Stat Med. 2008; 27:2420-2439). The BLRM estimates the MTD by updating estimates of the probability of observing a DLT in the MTD evaluation period for each dose level in the trial as patient information becomes available, taking into account updated DLT information from both schedules. At any time in the trial, it will not be permitted to escalate to a dose which does not fulfil the escalation with overdose control (EWOC) principle. Dose escalation is restricted to a maximum increment of 100% from the previous dose. Dose escalation and cohort size are based on decisions of the Dose Escalation Committee (DEC), guided by the BLRM.

The dose escalation part of the trial tests two different dosing schedules for compound (1) within one BLRM with a covariate (which will discriminate between BID and QD). In the BID Schedule the cycles are 3 weeks in duration and compound (1) are administered twice daily (BID), while in the QD Schedule the cycles are 3 weeks in duration and compound (1) is administered once daily (QD). The trial starts with the BID Schedule; after one dose level above predicted human therapeutic dose is determined safe by the DEC, the QD Schedule is initiated. When this QD cohort is considered safe, next BID dose level is opened.

After this moment, all dose level cohorts are opened alternating between BID and QD Schedules. BID dose cohort is filled first, and then drop down to the equivalent QD cohort. Successive cohorts of patients receive increasing doses of compound (1) until the MTD is reached. After all patients in a cohort have either experienced a DLT or have been observed for 21 days without having experienced DLTs, the BLRM is updated with the newly accumulated data from both schedules. The overdose risk is then calculated for each dose and dose escalation is permitted to all doses which fulfil the EWOC criterion. For each dosing schedule, based on the model and on additional information (PK, pharmacodynamics, patient profiles, information from the other dosing schedule), the members of the DEC reach a joint decision on the next dose level to be investigated and the size of the next cohort. Pre-specified dose levels are provisional and intermediate levels may be explored as deemed necessary by the DEC.

All cohorts include at least 3 patients. In the case that only two patients in a cohort are evaluable (i.e. one patient is not evaluable) and neither has experienced a DLT within the MTD evaluation period, dose-escalation can occur based on these two patients.

If DLTs are observed in the first two consecutive patients of a previously untested dose level, subsequent enrolment to that cohort is stopped. The BLRM is updated to confirm that the dose level still fulfils the EWOC principle. Based on this information, the DEC evaluates whether the next patients are enrolled at the same dose level, or if they are enrolled at a lower dose level. No further dose escalation takes place after the criterion for MTD is fulfilled. Further patients may be included to confirm this MTD estimate, i.e. to confirm that the EWOC criterion is still fulfilled. The DEC can declare any dose fulfilling the EWOC criterion as the RP2D, independent of the MTD estimate. The RP2D will not exceed the MTD. Any DLTs occurring after the MTD evaluation period are considered for the evaluation of the RP2D for compound (1). If no DLT is observed, the DEC may decide to declare the RP2D based on PK/Pharmacodynamic endpoints and overall safety profile. MTDs and RP2Ds are defined for both schedules separately.

At the end of dose escalation, when selected dose(s) for dose expansion are declared, dose escalation remains open for inclusion of patients not eligible for dose expansion, at selected dose(s), in sites participating in dose escalation, if agreed with DEC.

The RP2D(s) of compound (1) as monotherapy administered BID and QD is/are defined based on DLT/MTD (if reached), all safety data, and, if data allows, PK, PK/PD collected during the study. Should DLT or MTD not be reached, the RP2D(s) is/are determined based on safety data (i.e. overall tolerance and incidence of severe toxicity) as well as if data allows, PK, PK/PD.

The RP2D does not need to be the same in the two schedules (BID, QD). The BLRM is run based on extended data including all DLT-like events during the whole treatment period, as well as per treatment schedule alone, to further guide the selection of the RP2D(s).

The RP2D may be defined and the dose expansion part started before the MTD has been reached/dose escalation has concluded.

Patients may continue to receive treatment with compound (1) until disease progression (PD) according to RECIST or until another reason requiring termination of treatment (see Section 3.3.4).

### 8.1.4 Dose:

The starting doses of the dose escalation part of the trial are:
BID Schedule: 15 mg twice daily
QD Schedule: 60 mg once daily (if not proposed otherwise by the DEC).

Dose-escalation steps are determined by the DEC.

The predicted human dose for compound (1) was derived from a quantitative pharmacokinetic/tumour growth inhibition (PK/TGI) model. This preclinical mathematical model was built with input data from internal in vitro and in vivo data from efficacy experiments in PC-9 YVMA xenograft. Plasma exposure and tumor growth inhibition data from mice were used to train the model. The predicted human PK parameters were used to predict the human plasma profile in the preclinical PK/TGI model. In this setting the PK/TGI model was used to identify the required dose in human necessary to achieve a TGI >100%. This was predicted at 40 mg BID.

### 8.1.5 Patients

In the dose escalation part, patients with advanced, unresectable and/or metastatic solid tumours who are either refractory after standard therapy for the disease or for whom standard therapy is not appropriate are eligible. Patients must also have exhausted treatment options known to prolong survival for their disease. These patients should show a confirmed positive diagnosis as well of a HER2 aberration (described as overexpression according to standard diagnostic criteria OR gene amplification according to standard diagnostic criteria OR non-synonymous somatic mutation OR a gene rearrangement involving HER2 or NRG1).

Based on the provisional dose levels and escalation scheme, it is planned to enrol approximately 66 patients (around 36 patients for BID and around 30 patients for the QD dosing schedule) in the dose escalation part of the trial. The total number of patients depends on the number of dose escalations necessary.

All patients, including those who are eligible for the study based on local testing, must provide a tumor sample for confirmation of their HER2 status.

### Main Inclusion Criteria:

Patients with a confirmed diagnosis of an advanced, unresectable and/or metastatic non-haematologic malignancy with at least one measurable or evaluable lesion. Patient must show presence of at least one measurable lesion according to RECIST 1.1.
Eastern Cooperative Oncology Group score of 0 or 1
Availability and willingness to provide a sample of archival formalin-fixed paraffin embedded (FFPE) tumour tissue material for confirmation of the patient's HER2 status.
Patients must be willing and able to comply with the blood sampling and tumour biopsy requirements for PK, pharmacodynamics (PD) and biomarker analyses.
Adequate organ function as routinely measured in the field.
Recovered from any previous therapy-related toxicity to ≤Common Terminology Criteria for Adverse Events (CTCAE) Grade 1 at start of treatment (except for alopecia, stable sensory neuropathy and hypothyroidism (patients on thyroid replacement therapy) which must be ≤CTCAE Grade 2).
Life expectancy of at least 12 weeks at the start of treatment in the opinion of the investigator.
At least 18 years of age at the time of consent or over the legal age of consent in countries where that is greater than 18 years.
Signed and dated written informed consent in accordance with International Council on Harmonisation-Good Clinical Practice (ICH-GCP) and local legislation prior to admission to the trial.
Male or female patients. Women of childbearing potential (WOCBP) and men who are able to father a child must be ready and able to use highly effective methods of birth control per ICH M3 (R2) that result in a low failure rate of less than 1% per year when used consistently and correctly.
Patients with a documented aberration of the HER2 gene comprising: EITHER overexpression by immunohistochemistry (IHC), gene copy-number increase by in-situ hybridization (ISH), non-synonymous gene mutation OR gene fusion of the HER2 or NRG-1 genes.
Patient who has failed conventional treatment or for whom no therapy of proven efficacy exists or who is not eligible for established treatment options. Patient must have exhausted, or not be a suitable candidate for, available treatment options known to prolong survival for their disease.

Patients may discontinue trial treatment or withdraw consent to trial participation as a whole.

### 8.1.6 Compound:

Compound (1) is administered as film-coated tablets. This formulation was developed in three dosage strengths: 5 mg (about 10 mm round), 20 mg (about 10 mm round) and 100 mg (oval, about 16×7 mm). In addition to the drug substance, the tablets contain standard pharmaceutical excipients in common amounts.

### 8.1.7 Assessment of Efficacy:

Tumour assessments should include computed tomography (CT) scans of the chest, abdomen/pelvis (or PET/CT) and a brain MRI at screening. If clinically indicated, imaging of any other known or suspected sites of disease (e.g. bone) using an appropriate method (CT scan, MRI, PET/CT, or bone scan) should be performed. The same radiographic procedure must be used throughout the trial. Assessments will be performed by the investigator at screening (≤28 days prior to initiation of treatment), every 2 cycles (6 weeks ±5 days), at the end-of-treatment (EOT) visit (if not performed within the previous 3 weeks), and at the discretion of the investigator, and copies may be collected by the sponsor or designee. Wherever possible the assessment schedule should not be changed, but if there is an interruption or delay to treatment, alteration of the tumour assessment schedule to align with clinical assessments is allowed. Additional unscheduled tumour assessments may be performed at the discretion of the investigator. If the patient stops trial medication for a reason other than progression, tumour assessment according to RECIST v1.1 continues until progression (or until one of the following occurs; death, lost to follow-up, end of the trial).

Patient's clinical status is assessed locally by each investigator. The clinical status decline must be attributed to the underlying tumor progression and not due to co-morbidity or concomitant medication. In case of tumor-related clinical deterioration, every effort should be made to confirm the disease progression by imaging tests.

Tumour response is evaluated according to RECIST Version 1.1 (Eisenhauer E A, Therasse P, Bogaerts J, Schwartz L H, Sargent D, Ford R, et al. New response evaluation criteria in solid tumours: revised RECIST guideline (version 1.1). Eur J Cancer. 2009; 45:228-247). RECIST 1.1 is used for a) whole-body assessment (classical RECIST 1.1) and b) the tumour assessment of non-CNS areas. The assessment according to classical RECIST 1.1 by the investigator and/or the local radiologist is the basis for continuation or discontinuation of the trial in an individual patient (in addition to safety). No whole-body RECIST assessment is performed in the dose escalation part.

Baseline imaging should include imaging of all known or suspected sites of disease using an appropriate method. The investigator (or designee) records the target and non-target lesions in the Case Report Form (CRF or eCRF). Lesions in previously irradiated areas may not be considered measurable at baseline unless the lesions occurred after irradiation. The same method of assessment and the same imaging technique must be used at each subsequent timepoint to characterise each reported lesion throughout treatment and during follow-up.

### 8.1.8 Assessment of Safety:

Physical examinations including the measurements of height (screening only) and weight are done at screening, on Day 1 of each treatment cycle, at the EOT visit, and at the 30-day safety follow-up visit. However, patients have an abbreviated physical examination (focused on the specific disease, at the investigator's discretion) on Cycle 1 Day 1 (in case that previous physical examination is done within 72 hours of initiation of treatment), and Cycle 1 Day 15. A full physical examination serves to assess general health status and also as a clinical tumour assessment and may include but not limited to a cardiopulmonary examination, examination of the regional lymph nodes, the abdomen and an assessment of the mental and neurological status. Additional symptoms which have not been reported during a previous examination should be clarified. Wherever possible the same investigator should perform this examination.

A limited physical examination should include a cardiopulmonary examination, a clinical tumour assessment, an examination of the regional lymph nodes and an examination of the abdomen.

### 8.1.9 Assessment of Adverse Events:

An AE is defined as any untoward medical occurrence in a patient or clinical investigation subject administered a medicinal product and which does not necessarily have to have a causal relationship with this treatment.

An AE can therefore be any unfavourable and unintended sign (including an abnormal laboratory finding), symptom, or disease temporally associated with the use of a medicinal product, whether or not considered related to the medicinal product.

A serious adverse event (SAE) is defined as any AE, which fulfills at least one of the following criteria:
results in death,
is life-threatening, which refers to an event in which the patient was at risk of death at the time of the event; it does not refer to an event that hypothetically might have caused death if more severe,
requires inpatient hospitalisation or prolongation of existing hospitalisation
results in persistent or significant disability or incapacity,
is a congenital anomaly/birth defect,
is deemed serious for any other reason if it is an important medical event when based on appropriate medical judgement which may jeopardise the patient and may require medical or surgical intervention to prevent one of the other outcomes listed in the above definitions. Examples of such events are intensive treatment in an emergency room or at home for allergic bronchospasm, blood dyscrasias or convulsions that do not result in hospitalisation or development of dependency or abuse.

Medical judgement should be used to determine whether there is a reasonable possibility of a causal relationship between the adverse event and the BI investigational compound, considering all relevant factors, including pattern of reaction, temporal relationship, de-challenge or re-challenge, confounding factors such as concomitant medication, concomitant diseases and relevant history.

Arguments that may suggest that there is a reasonable possibility of a causal relationship could be:
The event is consistent with the known pharmacology of the drug.
The event is known to be caused by or attributed to the drug class.
A plausible time to onset of the event relative to the time of drug exposure.
Evidence that the event is reproducible when the drug is re-introduced.
No medically sound alternative aetiologies that could explain the event (e.g. pre-existing or concomitant diseases, or co-medications).
The event is typically drug-related and infrequent in the general population not exposed to drugs (e.g. Stevens-Johnson syndrome).
An indication of dose-response (i.e. greater effect size if the dose is increased, smaller effect size if dose is reduced).

Arguments that may suggest that there is no reasonable possibility of a causal relationship could be:
No plausible time to onset of the event relative to the time of drug exposure is evident (e.g. pre-treatment cases, diagnosis of cancer or chronic disease within days/weeks of drug administration; an allergic reaction weeks after discontinuation of the drug concerned).
Continuation of the event despite the withdrawal of the medication, taking into account the pharmacological properties of the compound (e.g. after 5 half-lives).
Of note, this criterion may not be applicable to events whose time course is prolonged despite removing the original trigger.
Additional arguments amongst those stated before, like alternative explanation (e.g. situations where other drugs or underlying diseases appear to provide a more likely explanation for the observed event than the drug concerned).
Disappearance of the event even though the trial drug treatment continues or remains unchanged.

The investigator maintains and keeps detailed records of all AEs in the patient files.

### 8.1.10 Assessment of Pharmacokinetics:

Pharmacokinetic (PK) profiles of compound (1) are investigated after the first and after repeated doses. Standard PK parameters are calculated, if data allows and if scientifically reasonable. Individual concentration data and the PK parameters calculated thereof are tabulated and graphically displayed. Statistical analyses are performed. A patient's PK data are flagged and excluded from the statistical analyses in case of protocol violations relevant to the evaluation of PK (to be decided no later than in the Report Planning Meeting or in case of PK non-evaluability (as revealed during data analysis, based on the criteria specified below). Reasons for exclusion of a patient's data are documented in the Clinical Trial Report (CTR).

If data allows, the pharmacokinetic parameters Cₘₐₓ (_{,ss}), AUC₀₋ₜ2 (_{,ss}), of compound (1) are assessed in terms of dose proportionality, for attainment of steady state. If deemed necessary, further PK parameters might be used for these assessments.

Preliminary PK analyses can be performed as necessary for DEC decisions. The final preliminary analysis are performed at the end of the dose escalation part prior to proceeding to the dose expansion part. In contrast to the final PK analysis, the preliminary analyses are based on planned sampling times rather than on actual times; no supplementary patient information, e.g. on AEs or concomitant medication, is used in these analyses, and the outputs are not validated. Minor discrepancies between preliminary and final results may therefore occur.

### 8.1.11 Compliance:

Patients are requested to bring all remaining trial medication including empty package material with them when attending visits.

Based on tablet counts, treatment compliance will be calculated as shown in the formula below. Doses missed in accordance with the protocol (e.g. dose interruption for AEs) will not be included in the calculation. Compliance will be verified by the Clinical Research Associate (CRA) authorised by the sponsor or delegate.

$$Treatment compliance ( \% ) = \frac{Number of tablets actually taken \times 100}{\begin{matrix}Number of tablets which should have \\ been take as directed by the investigator\end{matrix}}$$

A treatment compliance of 80-120% is considered good.

### 8.1.12 Statistical Methods:

Dose escalation is guided by a BLRM with overdose control (EWOC) that is fitted to binary toxicity outcomes (DLTs). The estimate of parameters is updated as data are accumulated using the BLRM. At the end of the dose escalation phase, the toxicity probability at each dose level are calculated to determine an estimate of the MTD.

### 8.1.13 Investigational Plan

All patients should adhere to the visit schedule. If treatment administration is delayed at any time, the schedule of all subsequent visits/cycles is recalculated based on the actual date of treatment. Visits may take place over more than one day for logistical reasons, as long as all assessments fall within the defined window.

Each visit and assessment are conducted in allowed windows. Additional flexibility (e.g. to allow for public holidays and patient unavailability) may be allowed if agreed between the investigator and the sponsor.

If a patient misses a visit, the visit should be rescheduled as soon as possible, and the delayed visit documented with the actual date and the reason for the delay. The scheduling of subsequent visits must not be altered, so if it is not possible to reschedule prior to the next planned visit, the missed visit should be skipped.

If a patient is hospitalised for administrative reasons to allow treatment and PK sampling this will not be considered an SAE, unless any other criteria for an SAE are fulfilled.

In addition to the scheduled assessments, unscheduled visits and unscheduled assessments for safety reasons may be performed at any time according to clinical need.

In the event of force majeure or other disruptive circumstances (e.g. pandemic, war) sites should adhere to the required protocol procedures as far as possible, however where a patient is unable or unwilling to attend a clinic visit, In situations where an individual patient is unable or unwilling to attend a clinic visit (because of force majeure or other disrupting circumstances such as pandemic, war) the investigator must assess the risk-benefit for the individual patient and may decide to perform a visit remotely if this is in the best interests of the patient and if agreed with the sponsor. All deviations from the original schedule of visits and procedures will be documented and the implications considered for the analysis of the trial data.

Following informed consent, the patient undergoes screening assessments. The assessments must fall within the acceptable Screening visit window but do not need to be performed on the same day. Screening assessments may be repeated as long as they fall within the Screening visit window. If more than one screening assessment is available, the latest assessment prior to the start of treatment must be used to assess eligibility.

If the patient meets the eligibility criteria during screening, the first treatment visit is scheduled. Any baseline conditions which are present at the Screening visit should be reported in the eCRF. Eligible patients are administered compound (1) daily until criteria for treatment discontinuation are met.

Patients may continue on treatment for unlimited cycles, until criteria for stopping treatment are met.

After a decision to permanently discontinue treatment within the trial is taken, no further administration of trial medication should take place and the EOT visit should be performed within 7 days of the decision. If the decision to permanently discontinue trial treatment is taken during a scheduled visit, the EOT visit should be performed instead of the scheduled visit.

In the dose escalation part of the trial, the follow-up visit (FU) is performed no less than 30 days after permanent discontinuation of compound (1) and is primarily to collect follow-up safety information. An individual patient who completes the follow up visit will be considered to have completed the trial.

### 8.2. Results:

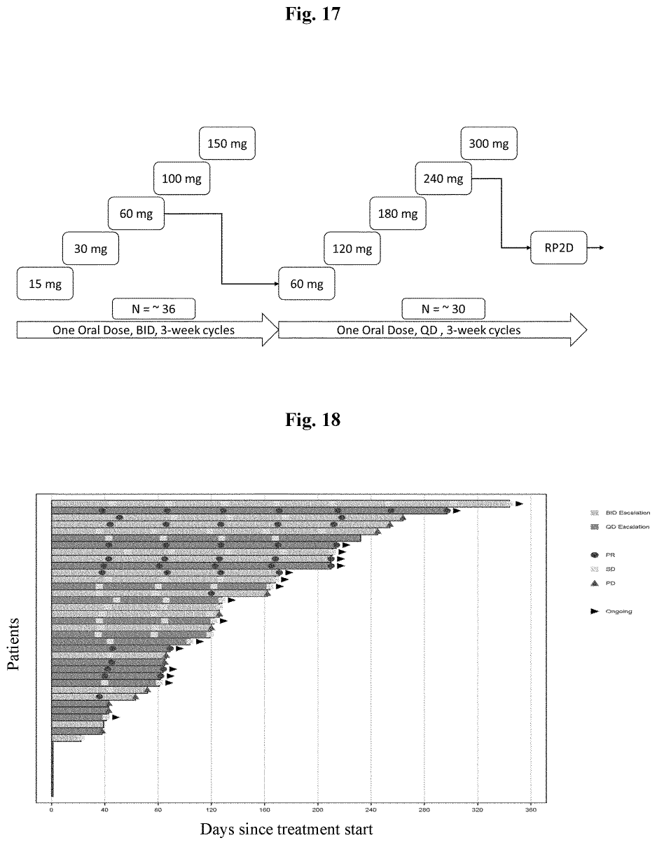

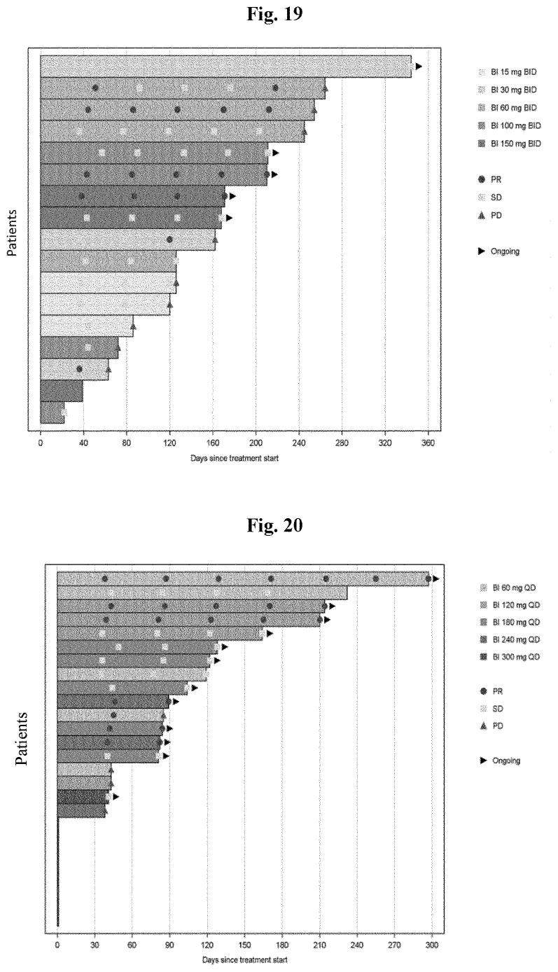

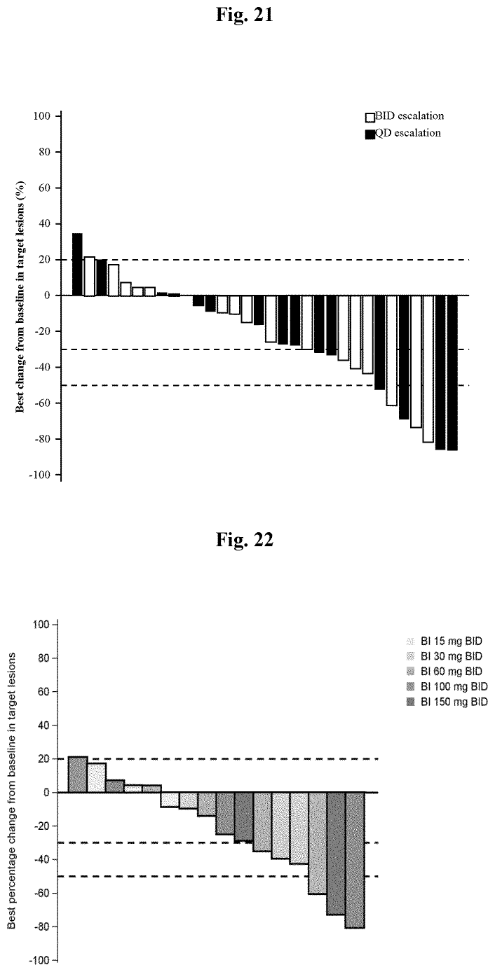

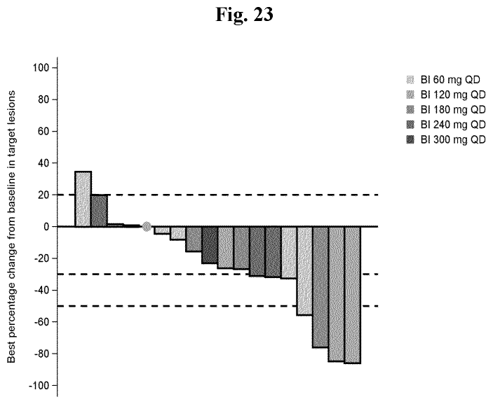

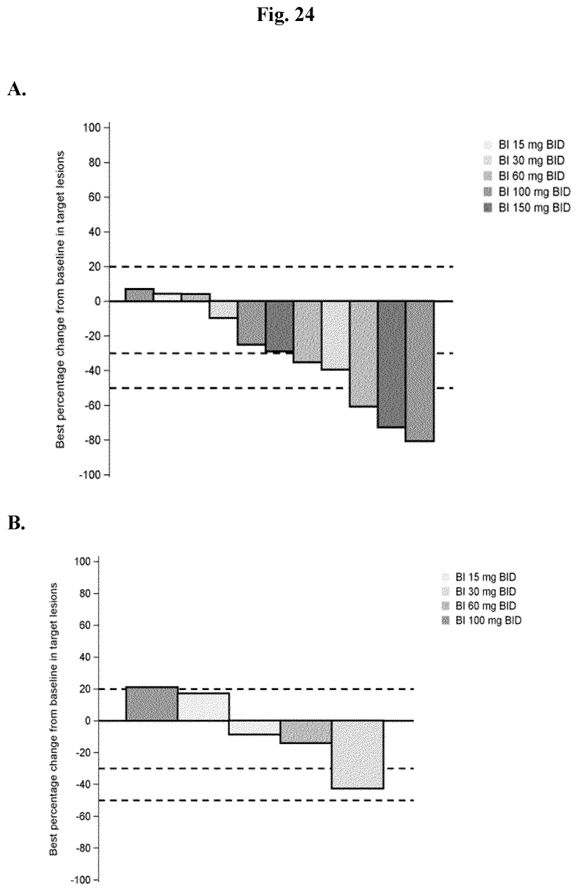

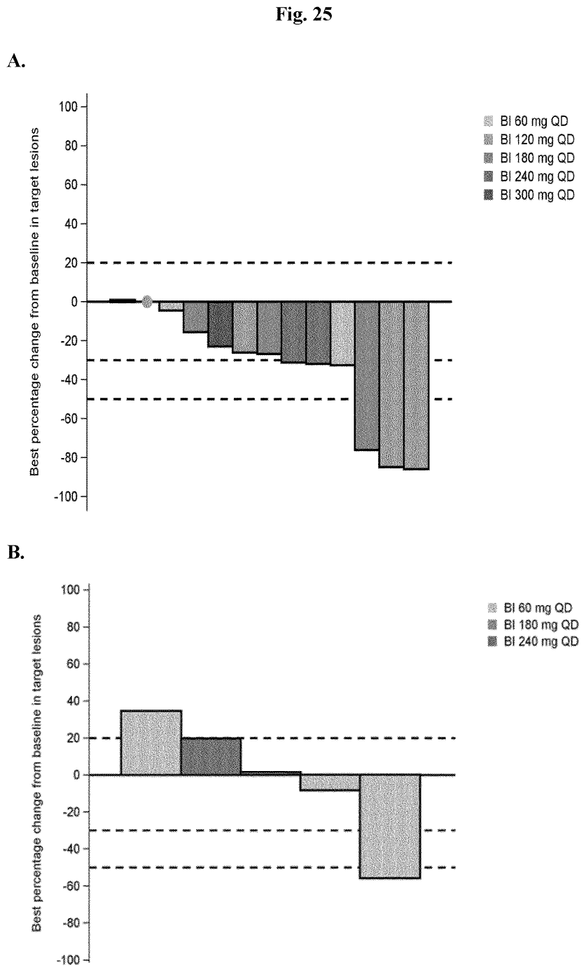

The study design is shown in FIG. 17. Data for the trial are summarised overall and pooled for the QD and BID schedules and for the dose selected for the dose expansion part of the trial.

### 8.2.1 Safety

As of the data lock point (9 Mar. 2023), safety data were available from 43 patients (Table 28). Patients were treated with escalating doses of compound (1) monotherapy administered using either a twice daily (BID, 17 patients) or once daily (QD, 26 patients) schedule. The starting dose was 15 mg twice daily in the BID schedule (N=3), with 30 mg BID (N=3), 60 mg BID (N=4), 100 mg BID (N=4), and 150 mg BID (N=3) also investigated. The starting dose was 60 mg once daily in the QD schedule (N=5), with 120 mg QD (N=4), 180 mg QD (N=6), 240 mg QD (N=6), and 300 mg QD (N=5) also investigated.

At the time of data lock, 17 of the 43 (39.5%) treated patients had discontinued trial treatment, 11 for objective disease progression, 3 for clinical disease progression, and 1 each for patient withdrawal and “other” reasons. Twenty-six patients were ongoing.

### 8.2.2 Exposure and Demographics

In total, 43 patients have been treated with compound (1). Overall, the median treatment duration at the data snapshot date was 138.1 days; exposure ranged from 9 to 416 days. The median number of treatment cycles initiated was 4.0 (range: 1 to 15).

Twenty-six patients (60.5%) were Asian and 17 patients (39.5%) were White. The median age of patients was 58 years (range 32 79); 23 (53.5%) patients were male. The main primary diagnosis was NSCLC (27 patients; 62.8%). The median time since first histological diagnosis was 34.46 months (range 7.2 to 137.7 months). All patients had metastatic disease at screening, with the predominant location of metastasis being lung, lymph node, and bone.

Among patients who were tested for HER2 (or NRG1 mutations), 10 out of 42 (23.3%) patients exhibited HER2 overexpression; 25 out of 42 (58.1%) patients exhibited non-synonymous somatic mutation, and 9 out of 42 (20.9%) patients exhibited HER2 or NRG1 gene rearrangements.

### 8.2.3 Overview of Safety

Across all doses and schedules, as of the data cut-off of Mar. 9, 2023, no serious unexpected safety findings were observed and reported adverse events (AEs) were manageable. The observed AEs and considerations on DLTs are summarised below.

A total of 38 of the 43 patients (88.4%) experienced one or more AEs. For 28 patients (65.1%), at least 1 AE was considered to be treatment-related by the investigator. Two patients experienced AEs leading to dose reduction of compound (1).

The most common AEs were diarrhoea (15 patients, 34.9%), followed by ALT increased (8 patients, 18.6%), anaemia (8 patients, 18.6%), and blood creatinine increased (7 patients, 16.3%).

In total, 13 patients (36.1%) had AEs of Grade 1, 8 patients (22.2%) of Grade 2, 9 patients (25.0%) of Grade 3, 1 patient (2.8%) of Grade 4, and 4 patients (11.1%) of Grade 5. No correlation between occurrence of AEs with ≥Grade 2 and dose could be detected in the data. By the preferred term (PT), Grade 3 events were ALT increased (4 patients, 11.1%), AST increased (2 patients, 5.6%), anaemia (2 patients, 5.6%), pneumonia, GGT increased, hypocalcaemia, pain in extremity, pleural effusion, atrial flutter, basal cell carcinoma, lymphocyte count decreased, pericardial effusion, and sepsis (1 patient each, 2.8%). One patient (2.8%) had a Grade 4 event (COVID-19). All Grade 5 (i.e., fatal) events were events related to the underlying cancer disease (malignant neoplasm progression: 2 patients, 5.6%; disease progression: 1 patient, 2.8%; lung neoplasm malignant: 1 patient, 2.8%)

In total, 16 patients (44.4%) had drug-related AEs of Grade 1, 6 patients (16.7%) of Grade 2, 3 patients (8.3%) of Grade 3, 0 patients of Grade 4, and 0 patients of Grade 5. Patients with drug-related AEs Grade 3 at time of data lock date were: a patient with drug-related AST and ALT increase Grade 3, a patient with drug-related ALT increase Grade 3, and a patient with anaemia Grade 3, hypocalcaemia Grade 3, GGT increased Grade 3, and lymphocyte count decreased Grade 3, all assessed as drug-related.

Overall, 11 patients (30.6%) had a serious adverse event (SAE).

Eight of the 11 patients had SAEs that were considered related to the underlying disease or an extraneous cause (malignant neoplasm progression in 1 patient, malignant neoplasm progression and basal cell carcinoma in 1 patient, disease progression in 1 patient, lung neoplasm malignant and metastases to central nervous system in 1 patient, pericardial effusion, pneumonia, and pleural effusion in 1 patient, sepsis in 1 patient, and ALT increased in 1 patient, and anaemia in 1 patient.

In addition, 3 patients had SAEs, where the investigator did not report a relationship to the underlying disease or an extraneous cause. These SAEs were: Grade 2/3 atrial flutter in 1 patient, Grade 2 pneumonia in 1 patient, and Grade 4 (life-threatening) COVID-19 in 1 patient. All 3 patients recovered from the SAE

At time of data lock, 4 patients had experienced a dose limiting toxicity: one of these patients (QD 60 mg) had an AE that was erroneously reported as DLT by the investigator (not drug-related Grade 3 anaemia on Day 213); the error has not been corrected yet at the time of the data snapshot. One patient (60 mg BID cohort) had Grade 2 oedema on Day 58 (drug-related, not serious). One patient (150 mg BID cohort) had Grade 2 diarrhoea on Day 155 (drug-related, not serious). One patient (180 mg QD cohort) had Grade 3 ALT increased (drug-related, not serious) and Grade 2 ALT increased on Day 84 (drug-related, not serious).

No DLTs were reported in 240 mg QD and 300 mg QD dose cohorts either in Cycle 1 or beyond. None of the patients in the dose escalation part of the trial experienced a DLT during the MTD evaluation period (first cycle; 21 days), with doses administered up to 150 mg BID and 300 mg QD. Thus, the MTD has not been reached in either of the two schedules with a total of 43 patients treated.

TABLE 28
Overall summary of adverse events during on-treatment period, cohort
BID escalation, treated set at the data lock of 17 Mar. 2023.

| | Compound (1) Dose in mg | | | | | | | | | | | |
|---|---|---|---|---|---|---|---|---|---|---|---|---|
| | 15 | | 30 | | 60 | | 100 | | 150 | | | |
| | BID | | | | | | | | | | Total | |
| | N | % | N | % | N | % | N | % | N | % | N | % |
| Number of | 3 | 100.0 | 3 | 100.0 | 4 | 100.0 | 4 | 100.0 | 3 | 100.0 | 17 | 100.0 |
| patients | | | | | | | | | | | | |
| Patients with any | 2 | 66.7 | 3 | 100.0 | 4 | 100.0 | 4 | 100.0 | 3 | 100.0 | 16 | 94.1 |
| AE | | | | | | | | | | | | |
| Patients with | 2 | 66.7 | 2 | 66.7 | 4 | 100.0 | 3 | 75.0 | 2 | 66.7 | 13 | 76.5 |
| investigator | | | | | | | | | | | | |
| defined drug- | | | | | | | | | | | | |
| related AEs | | | | | | | | | | | | |
| Patients with | 0 | 0.0 | 0 | 0.0 | 1 | 25.0 | 0 | 0.0 | 1 | 33.3 | 2 | 11.8 |
| protocol- | | | | | | | | | | | | |
| specified adverse | | | | | | | | | | | | |
| events of special | | | | | | | | | | | | |
| interest (AESI) | | | | | | | | | | | | |
| Patients with | 0 | 0.0 | 0 | 0.0 | 1 | 25.0 | 0 | 0.0 | 0 | 0.0 | 1 | 5.9 |
| AEs leading to | | | | | | | | | | | | |
| dose reduction of | | | | | | | | | | | | |
| Compound (1) | | | | | | | | | | | | |
| Patients with | 0 | 0.0 | 0 | 0.0 | 0 | 0.0 | 0 | 0.0 | 0 | 0.0 | 0 | 0.0 |
| AEs leading to | | | | | | | | | | | | |
| discontinuation | | | | | | | | | | | | |
| of Compound (1) | | | | | | | | | | | | |
| Patients with | 0 | 0.0 | 1 | 33.3 | 2 | 50.0 | 0 | 0.0 | 2 | 66.7 | 5 | 29.4 |
| AEs leading to | | | | | | | | | | | | |
| interruption of | | | | | | | | | | | | |
| Compound (1) | | | | | | | | | | | | |
| Patients with | 0 | 0.0 | 0 | 0.0 | 1 | 25.0 | 0 | 0.0 | 1 | 33.3 | 2 | 11.8 |
| DLTs | | | | | | | | | | | | |
| Patients with | 1 | 33.3 | 2 | 66.7 | 2 | 50.0 | 0 | 0.0 | 2 | 66.7 | 7 | 41.2 |
| serious AEs | | | | | | | | | | | | |
| Death | 1 | 33.3 | 1 | 33.3 | 0 | 0.0 | 0 | 0.0 | 1 | 33.3 | 3 | 17.6 |
| Life threatening | 0 | 0.0 | 0 | 0.0 | 1 | 25.0 | 0 | 0.0 | 0 | 0.0 | 1 | 5.9 |
| Disability or | 0 | 0.0 | 0 | 0.0 | 0 | 0.0 | 0 | 0.0 | 0 | 0.0 | 0 | 0.0 |
| permanent | | | | | | | | | | | | |
| damage | | | | | | | | | | | | |
| Hospitalization | 0 | 0.0 | 2 | 66.7 | 1 | 25.0 | 0 | 0.0 | 1 | 33.3 | 4 | 23.5 |
| (initial or | | | | | | | | | | | | |
| prolonged) | | | | | | | | | | | | |
| Congenital | 0 | 0.0 | 0 | 0.0 | 0 | 0.0 | 0 | 0.0 | 0 | 0.0 | 0 | 0.0 |
| anomaly or birth | | | | | | | | | | | | |
| detect | | | | | | | | | | | | |
| Other serious | 1 | 33.3 | 0 | 0.0 | 0 | 0.0 | 0 | 0.0 | 1 | 33.3 | 2 | 11.8 |
| (important | | | | | | | | | | | | |
| medical events) | | | | | | | | | | | | |
| Worst CTCAE | | | | | | | | | | | | |
| grade | | | | | | | | | | | | |
| Grade 1 | 0 | 0.0 | 0 | 0.0 | 1 | 25.0 | 3 | 75.0 | 0 | 0.0 | 4 | 23.5 |
| Grade 2 | 1 | 33.3 | 1 | 33.3 | 1 | 25.0 | 1 | 25.0 | 1 | 33.3 | 5 | 29.4 |
| Grade 3 | 0 | 0.0 | 1 | 33.3 | 1 | 25.0 | 0 | 0.0 | 1 | 33.3 | 3 | 17.6 |
| Grade 4 | 0 | 0.0 | 0 | 0.0 | 1 | 25.0 | 0 | 0.0 | 0 | 0.0 | 1 | 5.9 |
| Grade 5 | 1 | 33.3 | 1 | 33.3 | 0 | 0.0 | 0 | 0.0 | 1 | 33.3 | 3 | 17.6 |

TABLE 29
Overall summary of adverse events during on-treatment period, cohort
QD escalation, treated set at the data lock of 17 Mar. 2023.

| | Compound (1) Dose in mg | | | | | | | | | | | |
|---|---|---|---|---|---|---|---|---|---|---|---|---|
| | 60 | | 120 | | 180 | | 240 | | 300 | | | |
| | QD | | | | | | | | | | Total | |
| | N | % | N | % | N | % | N | % | N | % | N | % |
| Number of | 5 | 100.0 | 4 | 100.0 | 6 | 100.0 | 6 | 100.0 | 5 | 100.0 | 26 | 100.0 |
| patients | | | | | | | | | | | | |
| Patients with any | 5 | 100.0 | 4 | 100.0 | 5 | 83.3 | 5 | 83.3 | 3 | 60.0 | 22 | 84.6 |
| AE | | | | | | | | | | | | |
| Patients with | 3 | 60.0 | 2 | 50.0 | 4 | 66.7 | 4 | 66.7 | 2 | 40.0 | 15 | 57.7 |
| investigator | | | | | | | | | | | | |
| defined drug- | | | | | | | | | | | | |
| related AEs | | | | | | | | | | | | |
| Patients with | 1 | 20.0 | 0 | 0.0 | 1 | 16.7 | 1 | 16.7 | 0 | 0.0 | 3 | 11.5 |
| protocol- | | | | | | | | | | | | |
| specified adverse | | | | | | | | | | | | |
| events of special | | | | | | | | | | | | |
| interest (AESI) | | | | | | | | | | | | |
| Patients with | 0 | 0.0 | 0 | 0.0 | 0 | 0.0 | 1 | 16.7 | 0 | 0.0 | 1 | 3.8 |
| AEs leading to | | | | | | | | | | | | |
| dose reduction of | | | | | | | | | | | | |
| Compound (1) | | | | | | | | | | | | |
| Patients with | 1 | 20.0 | 0 | 0.0 | 1 | 16.7 | 0 | 0.0 | 0 | 0.0 | 2 | 7.7 |
| AEs leading to | | | | | | | | | | | | |
| discontinuation | | | | | | | | | | | | |
| of Compound (1) | | | | | | | | | | | | |
| Patients with | 0 | 0.0 | 2 | 50.0 | 1 | 16.7 | 0 | 0.0 | 0 | 0.0 | 3 | 11.5 |
| AEs leading to | | | | | | | | | | | | |
| interruption of | | | | | | | | | | | | |
| Compound (1) | | | | | | | | | | | | |
| Patients with | 1 | 20.0 | 0 | 0.0 | 1 | 16.7 | 0 | 0.0 | 0 | 0.0 | 2 | 7.7 |
| DLTs | | | | | | | | | | | | |
| Patients with | 2 | 40.0 | 2 | 50.0 | 0 | 0.0 | 2 | 33.3 | 1 | 20.0 | 7 | 26.9 |
| serious AEs | | | | | | | | | | | | |
| Death | 2 | 40.0 | 0 | 0.0 | 0 | 0.0 | 0 | 0.0 | 0 | 0.0 | 2 | 7.7 |
| Life threatening | 0 | 0.0 | 0 | 0.0 | 0 | 0.0 | 0 | 0.0 | 0 | 0.0 | 0 | 0.0 |
| Disability or | 0 | 0.0 | 0 | 0.0 | 0 | 0.0 | 0 | 0.0 | 0 | 0.0 | 0 | 0.0 |
| permanent | | | | | | | | | | | | |
| damage | | | | | | | | | | | | |
| Hospitalization | 2 | 40.0 | 2 | 50.0 | 0 | 0.0 | 0 | 0.0 | 1 | 20.0 | 5 | 19.2 |
| (initial or | | | | | | | | | | | | |
| prolonged) | | | | | | | | | | | | |
| Congenital | 0 | 0.0 | 0 | 0.0 | 0 | 0.0 | 0 | 0.0 | 0 | 0.0 | 0 | 0.0 |
| anomaly or birth | | | | | | | | | | | | |
| detect | | | | | | | | | | | | |
| Other serious | 0 | 0.0 | 1 | 25.0 | 0 | 0.0 | 2 | 33.3 | 0 | 0.0 | 3 | 11.5 |
| (important | | | | | | | | | | | | |
| medical events) | | | | | | | | | | | | |
| Worst CTCAE | | | | | | | | | | | | |
| grade | | | | | | | | | | | | |
| Grade 1 | 2 | 40.0 | 0 | 0.0 | 0 | 0.0 | 3 | 50.0 | 2 | 40.0 | 7 | 26.9 |
| Grade 2 | 0 | 0.0 | 2 | 50.0 | 3 | 50.0 | 1 | 16.7 | 0 | 0.0 | 6 | 23.1 |
| Grade 3 | 1 | 20.0 | 2 | 50.0 | 2 | 33.3 | 1 | 16.7 | 1 | 20.0 | 7 | 26.9 |
| Grade 4 | 0 | 0.0 | 0 | 0.0 | 0 | 0.0 | 0 | 0.0 | 0 | 0.0 | 0 | 0.0 |
| Grade 5 | 2 | 40.0 | 0 | 0.0 | 0 | 0.0 | 0 | 0.0 | 0 | 0.0 | 2 | 7.7 |

TABLE 30
Overall summary of adverse events during on-treatment period,
escalation, treated set at the data lock of 17 Mar. 2023.

| | BID | | QD | | Total | |
|---|---|---|---|---|---|---|
| | N | % | N | % | N | % |
| Number of patients | 17 | 100.0 | 26 | 100.0 | 43 | 100.0 |
| Patients with any AE | 16 | 94.1 | 22 | 84.6 | 38 | 88.4 |
| Patients with investigator defined drug-related AEs | 13 | 76.5 | 15 | 57.7 | 28 | 65.1 |
| Patients with protocol-specified adverse events of special interest | 2 | 11.8 | 3 | 11.5 | 5 | 11.6 |
| (AESI) | | | | | | |
| Patients with AEs leading to dose reduction of Compound (1) | 1 | 5.9 | 1 | 3.8 | 2 | 4.7 |
| Patients with AEs leading to discontinuation of Compound (1) | 0 | 0.0 | 2 | 7.7 | 2 | 4.7 |
| Patients with AEs leading to interruption of Compound (1) | 5 | 29.4 | 3 | 11.5 | 8 | 18.6 |
| Patients with DLTs | 2 | 11.8 | 2 | 7.7 | 4 | 9.3 |
| Patients with serious AEs | 7 | 41.2 | 7 | 26.9 | 14 | 32.6 |
| Death | 3 | 17.6 | 2 | 7.7 | 5 | 11.6 |
| Life threatening | 1 | 5.9 | 0 | 0.0 | 1 | 2.3 |
| Disability or permanent damage | 0 | 0.0 | 0 | 0.0 | 0 | 0.0 |
| Hospitalization (initial or prolonged) | 4 | 23.5 | 5 | 19.2 | 9 | 20.9 |
| Congenital anomaly or birth detect | 0 | 0.0 | 0 | 0.0 | 0 | 0.0 |
| Other serious (important medical events) | 2 | 11.8 | 3 | 11.5 | 5 | 11.6 |
| Worst CTCAE grade | | | | | | |
| Grade 1 | 4 | 23.5 | 7 | 26.9 | 11 | 25.6 |
| Grade 2 | 5 | 29.4 | 6 | 23.1 | 11 | 25.6 |
| Grade 3 | 3 | 17.6 | 7 | 26.9 | 10 | 23.3 |
| Grade 4 | 1 | 5.9 | 0 | 0.0 | 1 | 2.3 |
| Grade 5 | 3 | 17.6 | 2 | 7.7 | 5 | 11.6 |

### 8.2.4 Overview of Efficacy

At the time when tumour response assessments were available for 32 of 36 patients, 16 treated BID and 16 treated QD. Overall, the best tumor response of partial response (PR), regardless of confirmation, was reported for 12 patients. Stable disease was obtained in 17 patients; 3 patients had PD; for none of the patients a complete response (CR) was reported.

TABLE 31
Best overall response according to RECIST v1.1 (investigator
assessment), regardless of confirmation at the data lock of
17 Mar. 2023 - dose escalation, BID schedule, treated set.

| | Compound (1) in mg | | | | | | | | | | | |
|---|---|---|---|---|---|---|---|---|---|---|---|---|
| | 15 | | 30 | | 60 | | 100 | | 150 | | | |
| | BID | | | | | | | | | | Total | |
| | N | % | N | % | N | % | N | % | N | % | N | % |
| Number of | 3 | 100.0 | 3 | 100.0 | 4 | 100.0 | 4 | 100.0 | 3 | 100.0 | 17 | 100.0 |
| patients | | | | | | | | | | | | |

Best overall response (RECIST 1.1)

| | | | | | | | | | | | | |
|---|---|---|---|---|---|---|---|---|---|---|---|---|
| Complete | 0 | 0.0 | 0 | 0.0 | 0 | 0.0 | 0 | 0.0 | 0 | 0.0 | 0 | 0.0 |
| response | | | | | | | | | | | | |
| Partial | 0 | 0.0 | 2 | 66.7 | 2 | 50.0 | 1 | 25.0 | 1 | 33.3 | 6 | 35.3 |
| response | | | | | | | | | | | | |
| Stable | 3 | 100.0 | 1 | 33.3 | 2 | 50.0 | 3 | 75.0 | 1 | 33.3 | 10 | 58.8 |
| disease | | | | | | | | | | | | |
| Non- | 0 | 0.0 | 0 | 0.0 | 0 | 0.0 | 0 | 0.0 | 0 | 0.0 | 0 | 0.0 |
| CR/Non-PD | | | | | | | | | | | | |
| Progressive | 0 | 0.0 | 0 | 0.0 | 0 | 0.0 | 0 | 0.0 | 0 | 0.0 | 0 | 0.0 |
| disease | | | | | | | | | | | | |
| Not | 0 | 0.0 | 0 | 0.0 | 0 | 0.0 | 0 | 0.0 | 1 | 33.3 | 1 | 5.9 |
| evaluable | | | | | | | | | | | | |

TABLE 32
Best overall response according to RECIST v1.1 (investigator
assessment), regardless of confirmation at the data lock of
17 Mar. 2023 - dose escalation, QD schedule, treated set.

| | Compound (1) in mg | | | | | | | | | | | |
|---|---|---|---|---|---|---|---|---|---|---|---|---|
| | 60 | | 120 | | 180 | | 240 | | 300 | | | |
| | QD | | | | | | | | | | Total | |
| | N | % | N | % | N | % | N | % | N | % | N | % |
| Number of | 5 | 100.0 | 4 | 100.0 | 6 | 100.0 | 6 | 100.0 | 5 | 100.0 | 26 | 100.0 |
| patients | | | | | | | | | | | | |

Best overall response (RECIST 1.1)

| | | | | | | | | | | | | |
|---|---|---|---|---|---|---|---|---|---|---|---|---|
| Complete | 0 | 0.0 | 0 | 0.0 | 0 | 0.0 | 0 | 0.0 | 0 | 0.0 | 0 | 0.0 |
| response | | | | | | | | | | | | |
| Partial | 2 | 40.0 | 2 | 50.0 | 1 | 16.7 | 2 | 33.0 | 0 | 0.0 | 7 | 26.9 |
| response | | | | | | | | | | | | |
| Stable | 2 | 40.0 | 1 | 25.0 | 4 | 66.7 | 0 | 0.0 | 1 | 20.0 | 8 | 30.8 |
| disease | | | | | | | | | | | | |
| Non- | 0 | 0.0 | 0 | 0.0 | 0 | 0.0 | 0 | 0.0 | 0 | 0.0 | 0 | 0.0 |
| CR/Non-PD | | | | | | | | | | | | |
| Progressive | 1 | 20.0 | 1 | 25.0 | 0 | 0.0 | 1 | 16.7 | 0 | 0.0 | 3 | 11.5 |
| disease | | | | | | | | | | | | |
| Not | 0 | 0.0 | 0 | 0.0 | 1 | 16.7 | 3 | 50.0 | 4 | 80.0 | 8 | 30.8 |
| evaluable | | | | | | | | | | | | |

TABLE 33
Best overall response according to RECIST v1.1 (investigator assessment),
regardless of confirmation for NSCLC patients at the data lock of
16 Mar. 2023 - dose escalation, BID schedule, treated set

| | Compound (1) in mg | | | | | | | | | | | |
|---|---|---|---|---|---|---|---|---|---|---|---|---|
| | 15 | | 30 | | 60 | | 100 | | 150 | | | |
| | BID | | | | | | | | | | Total | |
| | N | % | N | % | N | % | N | % | N | % | N | % |
| Number of | 1 | 100.0 | 2 | 100.0 | 3 | 100.0 | 3 | 100.0 | 2 | 100.0 | 11 | 100.0 |
| patients | | | | | | | | | | | | |

Best overall response (RECIST 1.1)

| | | | | | | | | | | | | |
|---|---|---|---|---|---|---|---|---|---|---|---|---|
| Complete | 0 | 0.0 | 0 | 0.0 | 0 | 0.0 | 0 | 0.0 | 0 | 0.0 | 0 | 0.0 |
| response | | | | | | | | | | | | |
| Partial | 0 | 0.0 | 1 | 50.0 | 2 | 66.7 | 1 | 33.0 | 1 | 50.0 | 5 | 45.5 |
| response | | | | | | | | | | | | |
| Stable | 1 | 100.0 | 1 | 50.0 | 1 | 33.3 | 2 | 66.7 | 1 | 50.0 | 6 | 54.5 |
| disease | | | | | | | | | | | | |
| Non- | 0 | 0.0 | 0 | 0.0 | 0 | 0.0 | 0 | 0.0 | 0 | 0.0 | 0 | 0.0 |
| CR/Non-PD | | | | | | | | | | | | |
| Progressive | 0 | 0.0 | 0 | 0.0 | 0 | 0.0 | 0 | 0.0 | 0 | 0.0 | 0 | 0.0 |
| disease | | | | | | | | | | | | |
| Not | 0 | 0.0 | 0 | 0.0 | 0 | 0.0 | 0 | 0.0 | 0 | 0.0 | 0 | 0.0 |
| evaluable | | | | | | | | | | | | |

TABLE 34
Best overall response according to RECIST v1.1 (investigator assessment),
regardless of confirmation for NSCLC patients at the data lock of
16 Mar. 2023 - dose escalation, QD schedule, treated set

| | Compound (1) in mg | | | | | | | | | | | |
|---|---|---|---|---|---|---|---|---|---|---|---|---|
| | 60 | | 120 | | 180 | | 240 | | 300 | | | |
| | QD | | | | | | | | | | Total | |
| | N | % | N | % | N | % | N | % | N | % | N | % |
| Number of | 2 | 100.0 | 4 | 100.0 | 5 | 100.0 | 5 | 100.0 | 5 | 100.0 | 21 | 100.0 |
| patients | | | | | | | | | | | | |

Best overall response (RECIST 1.1)

| | | | | | | | | | | | | |
|---|---|---|---|---|---|---|---|---|---|---|---|---|
| Complete | 0 | 0.0 | 0 | 0.0 | 0 | 0.0 | 0 | 0.0 | 0 | 0.0 | 0 | 0.0 |
| response | | | | | | | | | | | | |
| Partial | 1 | 50.0 | 2 | 50.0 | 1 | 20.0 | 2 | 40.0 | 0 | 0.0 | 6 | 28.6 |
| response | | | | | | | | | | | | |
| Stable | 1 | 50.0 | 1 | 25.0 | 3 | 60.0 | 0 | 0.0 | 1 | 20.0 | 6 | 28.6 |
| disease | | | | | | | | | | | | |
| Non- | 0 | 0.0 | 0 | 0.0 | 0 | 0.0 | 0 | 0.0 | 0 | 0.0 | 0 | 0.0 |
| CR/Non-PD | | | | | | | | | | | | |
| Progressive | 0 | 0.0 | 1 | 25.0 | 0 | 0.0 | 0 | 0.0 | 0 | 0.0 | 1 | 4.8 |
| disease | | | | | | | | | | | | |
| Not | 0 | 0.0 | 0 | 0.0 | 1 | 20.0 | 3 | 60.0 | 4 | 80.0 | 8 | 38.1 |
| evaluable | | | | | | | | | | | | |

Further preliminary evidence of efficacy is provided in FIGS. 2 to 9. According to FIG. 2, 13 patients out of 35 (37.14%) that had been treated for at least one cycle of 21 days on Jan. 23, 2023 show at least one PR assessment. This increases to 7 patients out of 18 (38.89%) when considering the QD schedule, as shown in FIG. 4. In FIGS. 5 to 9, each bar represents a different patient. Bars represent patients whose index is sorted by maximum % decrease. Change from baseline was calculated as the difference between lesion size at screening and at the end of 2 cycles (Day 42). Negative values indicate a reduction in the sum of target lesions diameters and positive values an increase. Data depicted in FIGS. 6 to 9 are from Mar. 16, 2023, while data depicted in FIG. 5 are from Jan. 23, 2023.

### 8.2.5 Exposure

Plasma exposure increased with increasing doses with no apparent deviation from dose proportional pharmacokinetics. However, there was substantial intra and interpatient variability in pharmacokinetics observed with overlapping exposure between dose groups, e.g., at 180 mg QD, 240 mg QD and 300 mg QD. Flat exposure-response relationships were observed for tumor shrinkage and ORR, indicating no trend of substantial improvements of efficacy across the higher dose range (180 mg-300 mg).

Exposure-toxicity analyses were also conducted. In terms of toxicity endpoints, the occurrence of AEs of Grade 2 or higher and of AEs of Grade 3 or higher was considered. For all considered exposure endpoints, the general exposure-toxicity relationship considering all AEs is relatively flat, with no indication of a higher incidence of general AEs of Grade 2/3 (and higher) for patients with higher exposure. However, for specific AEs, e.g., diarrhoea AEs, a trend for a potentially higher incidence for higher exposure was observed.

### 8.2.6 Duration of Treatment

Twenty-four patients have been treated in the higher dose cohorts (100 mg BID and above and 180 mg QD and above) have been on treatment for relatively long durations to-date. For these patients, the median total treatment duration is currently >6 months for the 150 mg BID cohort and almost 5 months for the 100 mg BID and 180 mg QD cohorts, as shown below. Most of these patients are still on treatment as of 9 Mar. 2023.

| | | |
|---|---|---|
| 100 mg BID | 147 days | (2/4 pts still on treatment) |
| 150 mg BID | 185 days | (2/3 pts still on treatment) |
| 180 mg QD | 146 days | (6/6 pts still on treatment) |
| 240 mg QD | 72 days | (5/6 pts still on treatment) |
| 300 mg QD | 57 days | (5/5 pts still on treatment) |

Overall, the median treatment duration at the data snapshot date was 126.0 days. The median number of treatment cycles initiated was 4.0 (range: 1 to 15).

### 8.2.7 Duration of Response

Based on all treated patients, the median duration of response (Kaplan Meier estimates) was 7 months.

Based on all treated patients with NSCLC, the median duration of response (Kaplan Meier estimates) was 6.9 months (95% CI: 1.4 to 7.0).

Results presented herein are only preliminary, because many patients are still ongoing treatment.

8.2.8 Patients Pre-Treated with Trastuzumab Deruxtecan

At the data cut-off of 17 Jul. 2023, 50 patients had been treated. 5 (10.0%) patients had received trastuzumab deruxtecan prior to compound (1). These patients had NSCLC. Up to −53% tumour shrinkage (mean −22.5%, median −16.1%) was observed in these patients.

### Example 9—a Phase Ib Clinical Trial with the Solid Dispersion According to the Invention

This Example focuses on the dose expansion part (Phase Ib) of the Phase I study described in Example 8. Approximately 275 patients diagnosed with advanced or metastatic refractory NSCLC harbouring mutations in the HER2 gene are enrolled. Patients are divided into 5 cohorts according to the main inclusion criteria listed below.

### 9.1 Protocol

The trial objectives of Phase Ib include:
For cohort 1, cohort 2, and cohort 5, assess objective tumour response rate by central independent review;
For cohort 3, assess objective tumour response rate by investigator assessment;
For all cohorts, further investigate the safety, tolerability, and PK of the investigated doses of zongertinib in patients with HER2 mutation positive advanced/metastatic NSCLC;
For all cohorts, continue evaluating safety, and patient-reported outcomes.

The primary endpoints of Phase Ib include:
For cohort 1, 2, and 5: objective response (OR) assessed by independent central review, according to Response Evaluation Criteria In Solid Tumors (RECIST) version 1.1;
For cohort 3: objective response (OR) by investigator.

The secondary endpoints include:

For Cohort 1, 2, and 5:
Duration of objective response (DoR) according to RECIST 1.1 by central independent review;
Disease control (DC) according to RECIST 1.1 by central independent review;
Progression-free survival (PFS) according to RECIST 1.1 by central independent review;

For Cohort 3 only:
Duration of OR according to RECIST 1.1 by investigator assessment;
DC according to RECIST 1.1 as assessed by the investigator;
PFS according to RECIST 1.1 as assessed by the investigator;

For all cohorts:
Number of patients experiencing DLTs during the entire treatment period;
Change from baseline to C5D1 in EORTC QLQ-C30 physical functioning domain score;
Change from baseline to C5D1 in NSCLC-SAQ total score;
Change from baseline to C5D1 in EORTC IL46 item score.

The main inclusion criteria include:
Patients with a confirmed diagnosis of an advanced, unresectable and/or metastatic non-haematologic malignancy with at least one measurable lesion;
Availability and willingness to provide a sample of archival formalin-fixed paraffin embedded (FFPE) tumour tissue material;
Patients must be willing to comply with the blood sampling and tumour biopsy requirements for PK, PD and biomarker analyses;
For cohort 1: Patients with documented HER2 Tyrosine Kinase Domain (TKD) mutation positive non-squamous NSCLC who had received, in the advanced/metastatic setting, at least one line of systemic therapy that includes a platinum-based combination chemotherapy;
For cohort 2: Patients with documented HER2 Tyrosine Kinase Domain (TKD) mutation positive non-squamous NSCLC who have not received any previous lines of therapy (neo or adjuvant chemo, chemoradio or radiotherapy is permitted if at least 6 months have elapsed prior to disease progression);
For cohort 3: Patients with documented HER2 Non-TKD mutation positive NSCLC, and HER2 TKD mutation positive squamous NSCLC, who had received in the advanced/metastatic setting at least one line of systemic therapy that includes a platinum-based combination chemotherapy;
Patients in these cohorts 1 to 4 must have not received prior therapy with HER2 directed antibody-drug conjugates (ADC);
For cohort 5: Patients with documented HER2 TKD mutation positive non-squamous NSCLC who had received prior therapy with HER2 directed ADC in the advanced/metastatic setting and developed disease progression during or after completing this therapy.

Compound (1) is administered as the compositions of Examples 6.2-B or 6.2-C, orally, at a dose of 120 mg or 240 mg QD.

The treatment consists of reiterated cycles of 3 weeks as long as the patient has clinical benefit or until undue drug toxicity or withdrawal of consent, whichever occurs first.

For Cohort 1, the analysis of the primary endpoint OR by central independent review is performed with a one-sided z-test at the one-sided alpha level of 0.0125 (to account for the two doses investigated).

For Cohort 2, the analysis of the primary endpoint OR by central independent review is performed with a one-sided z-test at the one-sided alpha level of 0.025.

For Cohort 3, no confirmatory testing is done. Only descriptive analyses are performed, in terms of the observed ORR and 95% CIs by the Wilson method. In addition, the primary endpoint OR by investigator assessment is analysed using a Bayesian Hierarchical Model (BHM) for the ORR, hereby leveraging available co-data from Cohorts 1. 2, 3, and 4 in a meta-analytic Bayesian approach.

For Cohort 5, the analysis of the primary endpoint OR by central independent review will be performed with a one-sided z-test at the one-sided alpha level of 0.025.

### 9.2 Results

As of Jul. 31, 2023, 42 patients had been treated in Cohort 1 of Phase Ib (randomized to 120/240 mg QD). Treatment related adverse events (TRAEs) (all/grade ≥3) were observed in 67%/10% of patients. The most common TRAEs were diarrhea (29%/0%), rash (21%/0%), increased aspartate aminotransferase (10%/2%), low appetite (10%/0%) and dysgeusia (10%/0%). No AEs led to treatment discontinuation. ORR/DCR in 23 evaluable patients (received 2-5 cycles at cut-off) were 74%/91%. All responding patients remained on treatment at data cut-off.

### Reference Example 1: Manufacture of Crystalline Forms of Compound (1)

Form III and IV of compound (1) referred to in Example 5.1 can be produced according to the procedures given below. It should be noted that the input form of compound (1) is not strictly consequential for the crystallization procedures if full dissolution is achieved prior to crystallization. In case of full dissolution, the compound (1) starting material can for example be produced according to the synthesis described in WO 2021/213800.

### Reference Example 1.1—Manufacture of Form I

First example procedure for the preparation of form I (crystallization): 19 kg of compound (1) (any solid state form) are dissolved in a mixture of ~54 kg THF, ~160 kg DCM and ~48 kg MeOH. Residual inorganic salts are removed by washing with brine (48 kg). Undissolved particulates are removed by polish filtration of the organic layer. The organic layer is then distilled to ~160 L and the mixture is diluted with 78 kg of THF. The distillation, THE dilution, distillation sequence is repeated until levels of water and MeOH ≤1.0% w/w each. Upon completion of distillations, the obtained slurry is held at ambient temperature for not longer than 12 hrs and filtered to yield form I.

Second example procedure for the preparation of form I (crystallization): 6 g of form IV of compound (1) (e.g. prepared according to one of the Examples described herein) are dissolved in 75 g 5% w/w H₂O in IPA solution at 90° C. Solution is slowly cooled to 75° C. and seeded with 60 mg of form I. Mixture is agitated at 75° C. for 2 hours followed by cooling at 0.3° C./min rate to 20° C. Upon completion of cooling, solids are filtered and dried to yield form I.

### Reference Example 1.2—Manufacture of Form III

Example procedure for the preparation of form III (slurry): 17 kg of form I of compound (1) is mixed with 271 kg of IPAc. Slurry is heated to 70° C. To the slurry, 0.2 kg of seeds of form III of compound (1) (e.g. prepared according to one of the Examples described herein) are added, and mixture is agitated for ~16 hrs. Upon completion of hold, mixture is gradually cooled to 53° C. in ~40 mins, then, to 33° C. in ~40 mins, then to 25° C. Obtained slurry is agitated for ~1 hr and filtered. Solids are washed with 27 kg of IPAc and dried to yield form III.

The procedure can also be performed without addition of seeds.

### Reference Example 1.3—Manufacture of Form IV

First example procedure for the preparation of form IV (crystallization): 300 mg form I of compound (1) are dispersed in 3 ml of 1-BuOH. Mixture is heated to 90° C. with over-head agitation. Dissolution is observed. Solution is cooled with the rate of 0.2° C./min to 75° C. followed by quick cooling to 20° C. Obtained slurry is held for ~12 hrs at 20° C. while agitated and filtered to yield form IV.

Second example procedure for the preparation of form IV (crystallization): Form III of compound (1) is dissolved in 10 volumes of 1-BuOH/Anisole (1:1) mixture at 110° C. Solution is subjected to distillation with slight vacuum during which most of the 1-BuOH is removed. The solution is seeded with form III seeds, held at 110° C. and a slurry is obtained. Mixture is cooled to ambient temperature while agitated and filtered to yield isolated compound (1) as form IV. This despite being seeded with form III.

Third example procedure for the preparation of form IV (slurry): A slurry of form I and IV of compound (1) is slurried in IPAc at temperatures ranging from 25-75° C. for 72-168 hrs. Mixture is brought to ambient temperature if applicable and filtered to yield compound (1) as form IV.

### Reference Example 2—XRPD of Solid State Forms of Compound (1)

Crystalline forms of compound (1) were analysed by XRPD. XRPDs were measured at a temperature in the range of from 20 to 30° C. using CuK_{α} radiation having a wavelength of 1.54184 Å.

The methodology for this analysis was as follows: the respective solid compound (approximately 0.2 g) is representatively subsampled into a stainless-steel sample holder fitted with a zero-diffraction plate (ZDP). The sample holder is then levelled off with a glass slide to create a flat sample surface level with the sample holder. The instrument used for the analysis is a Bruker D2 Phaser (system EQ-SSRD-XRD-01). A corundum reference standard is run each day to evaluate the system performance. Two peaks must be within ±0.02° 20 for system suitability to be acceptable. The instrument settings for the measurement of the solid compound samples can be seen in Table 35. Processing (Kα2 contribution stripping, peak labelling) was completed using DIFFRAC.EVA software (version 5.0).

In the following, experimental parameters for XRPD measurements are given:

TABLE 35
experimental parameters for XRPD measurements

| Parameter | Full range scan | Regions of interest scans |
|---|---|---|
| Model | Bruker D2 Phaser | Bruker D2 Phaser |
| X-ray radiation source | Cu Kα (λ = 1.54184 Å) | Cu Kα (λ = 1.54184 Å) |
| Current (mA) | 10 | 10 |
| Voltage (kV) | 30 | 30 |
| Scan type | Coupled Two Theta/Theta | Coupled Two Theta/Theta |
| Scan mode | Continuous PSD fast | Continuous PSD fast |
| Application type | Powder Diffraction | Powder Diffraction |
| Goniometer radius (mm) | 141.0 | 141.0 |
| Range (°2θ) | 5 to 30 | 6.75 to 8.75, 11.5 to 13.0, |
| | | 13.75 to 15.25, 18.0 to 19.75 |
| PSD opening (°) | 2.5 | 3.0 |
| Step size (°) | 0.01 | 0.014 |
| Rate (s/step) | 1.0 | 10.0 |
| Divergence slit width (mm) | 0.6 | 0.6 |
| Detector slit width (mm) | 3 | 3 |
| Air scatter screen (mm) | 1 | 1 |
| Rotation (rotations/minute) | 15 | 15 |

Table 36 lists the peaks for each form (that are above 5% relative intensity). Table 37 lists the best characteristic peaks to use when trying to identify a given polymorphic form where another form is present. The diagnostic peaks are indicative of peak positions where the impurity has a relatively high intensity peak, and the sample dominant form has a flat baseline.

TABLE 36
XRPD peak comparison

| Form I | Form III | Form IV |
|---|---|---|
| (°2θ) | (°2θ) | (°2θ) |
| 6.063 | — | 5.872** |
| — | 6.224* | — |
| 7.853** | — | — |
| — | 9.535* | — |
| 11.095* | — | — |
| — | 11.448* | 11.538** |
| — | — | 11.690** |
| 12.048** | 12.352** | — |
| 12.923* | — | — |
| — | 13.138 | — |
| — | 13.504* | — |
| 13.875 | 13.776* | — |
| — | 14.011* | — |
| — | — | 14.670* |
| 15.602 | 15.449 | — |
| — | 16.151* | — |
| — | 16.344 | — |
| — | — | 16.706 |
| 17.219* | — | — |
| 17.943** | — | — |
| — | 18.273* | — |
| 18.849* | — | 18.778* |
| — | — | 19.202 |
| — | 20.190* | |
| — | 20.621* | 20.463 |
| — | 21.079 | — |
| — | 21.267* | 21.349 |
| 21.521* | — | — |
| 22.213* | — | — |
| — | 22.852* | — |
| 23.090 | — | — |
| — | 23.492* | — |
| — | — | 24.300 |
| — | 24.540* | — |
| — | 25.126* | — |
| 25.891* | — | 25.796* |
| 26.491 | 26.417* | — |
| — | 27.210* | — |
| — | 29.068 | — |

With regard to Table 36, bolded peaks are determined to be characteristic peaks, peaks marked “*” have a greater than 10% relative intensity, peaks marked “**” have a greater than 50% relative intensity. Further, the peaks are listed in order of peak position (° 20), with similar peak positions on the same row.

TABLE 37
XRPD characteristic peaks (°2θ) for polymorphic impurity identification

| Form to be detected | Sample matrix | | |
|---|---|---|---|
| as impurity | In Form I | In Form III | In Form IV |
| Form I | — | 7.85, 17.22, 25.89 | 7.85, 11.10, 12.05 |
| Form III | 27.21, 16.15, 11.45 | — | 18.27, 6.22 |
| Form IV | 14.67, 11.69, 16.71 | 5.87, 18.78, 19.20, 25.80 | — |

1. A method for the treatment of cancer, comprising administering to a patient in need thereof a therapeutically effective amount of a solid dispersion comprising compound (1) as defined below or a pharmaceutically acceptable salt thereof 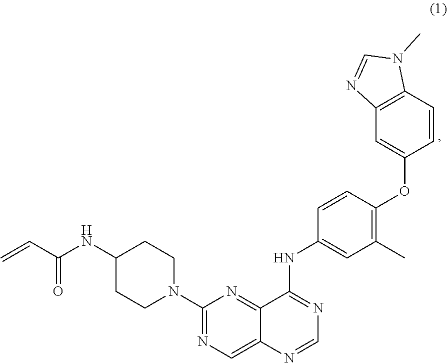 [SMILES: C=CC(=O)NC1CCN(c2ncc3ncnc(Nc4ccc(Oc5ccc6c(c5)ncn6C)c(C)c4)c3n2)CC1]
and a pharmaceutically acceptable dispersion carrier, wherein compound (1) or the pharmaceutically acceptable salt thereof is administered in a daily dose of at least 30 mg.

2. A method for the treatment of cancer, comprising administering to a patient in need thereof a therapeutically effective amount of a solid dispersion comprising compound (1) as defined below or a pharmaceutically acceptable salt thereof 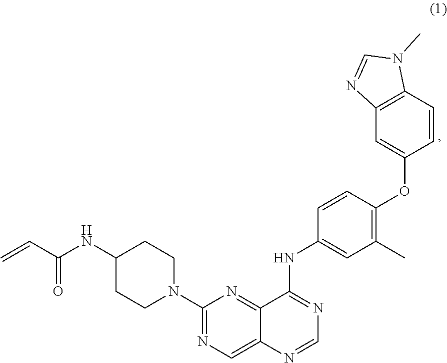 [SMILES: C=CC(=O)NC1CCN(c2ncc3ncnc(Nc4ccc(Oc5ccc6c(c5)ncn6C)c(C)c4)c3n2)CC1]
and a pharmaceutically acceptable dispersion carrier, wherein compound (1) or the pharmaceutically acceptable salt thereof is administered following administration of a systemic anti-cancer therapy agent.

3. The method according to claim 1, wherein the pharmaceutically acceptable dispersion carrier is a polymer.

4. The method according to claim 3, wherein the polymer is enteric or non-enteric.

5. The method according to claim 1, wherein the pharmaceutically acceptable dispersion carrier is a polymer selected from the group consisting of a hydroxypropyl methylcelluloses (or an ester thereof), a polyvinylpyrrolidone (or a copolymer thereof), a polymethacrylate (or a copolymer thereof), and a combination thereof.

6. The method according to claim 5, wherein the pharmaceutically acceptable dispersion carrier is the hydroxypropyl methylcelluloses (or ester thereof), wherein the hydroxypropyl methylcellulose (or ester thereof) is selected from the group consisting of hydroxypropyl methylcellulose acetate succinate and hot melt extrusion-grade hydroxypropyl methylcellulose.

7. The method according to claim 5, wherein the pharmaceutically acceptable dispersion carrier is the polyvinylpyrrolidone (or copolymer thereof) wherein the polyvinylpyrrolidone (or copolymer thereof) comprises a polyvinylpyrrolidone vinyl acetate copolymer.

8. The method according to claim 5, wherein the pharmaceutically acceptable dispersion carrier is the polymethacrylate (or copolymer thereof), wherein the polymethacrylate (or copolymer thereof) a comprises a methylacrylic acid methyl methacrylate copolymer.

9. (canceled)

10. The method according to claim 1, wherein compound (1) is amorphous.

11. The method according to claim 1, wherein compound (1) is present in an amount in a range of from 25 wt % to 75 wt %, based on a total weight of 100 wt % of the solid dispersion and the pharmaceutically acceptable dispersion carrier is present in an amount in a range of from 25 wt % to 75 wt %, based on a total weight % of the solid dispersion.

12. (canceled)

13. The method according to claim 1, wherein the weight ratio of compound (1) to the pharmaceutically acceptable dispersion carrier in the solid dispersion is of 1:1 to 1:3.

14. The method according to claim 1, wherein the solid dispersion is characterized by having an x-ray powder diffractogram comprising no diffraction peak at 2-theta angles equal or below 40.0°, when measured at a temperature in the range of from 20 to 30° C. and with Cu-Kα radiation having a wavelength of 1.54056 Å or 1.54184 Å.

15. A method for the treatment of cancer, comprising administering to a patient in need thereof a therapeutically effective amount of a pharmaceutical composition comprising a solid dispersion and one or more pharmaceutically acceptable excipients, wherein the solid dispersion comprises compound (1) as defined below or a pharmaceutically acceptable salt thereof 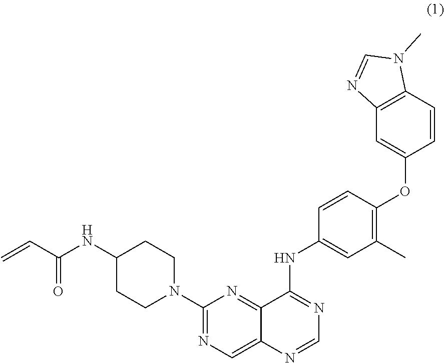 [SMILES: C=CC(=O)NC1CCN(c2ncc3ncnc(Nc4ccc(Oc5ccc6c(c5)ncn6C)c(C)c4)c3n2)CC1]
wherein compound (1) or the pharmaceutically acceptable salt thereof is administered in a daily dose of at least 30 mg.

16.-25. (canceled)

26. The method according to claim 1, wherein the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

27. The method according to claim 26, wherein said cancer is a HER2 overexpressed, HER2 amplified and/or HER2 mutant cancer.

28. The method according to claim 26, wherein said cancer is unresectable or metastatic cancer.

29.-31. (canceled)

32. The method according to claim 1, wherein compound (1) is administered in a daily dose of 30 mg to 600 mg.

33. (canceled)

34. (canceled)

35. The method according to claim 1, wherein compound (1) is administered once daily in a daily dose of 60 mg, 120 mg, 180 mg, 240 mg, 300 mg, 360 mg, 400 mg, 420 mg, 480 mg, 500 mg, 540 mg or 600 mg.

36. (canceled)

37. (canceled)

38. The method according to claim 1, wherein the cancer is advanced, unresectable or metastatic NSCLC harboring a HER2 mutation, wherein said HER2 mutation is in the tyrosine kinase domain.

39. The method according to claim 1, wherein the patient has already received one or more systemic anti-cancer therapy.

40. The method according to claim 1, wherein the cancer is non-small cell lung cancer (NSCLC).

41. The method according to claim 40, wherein compound (1) is administered once daily in a daily dose of 120 mg.

42. A method for the treatment of cancer, comprising administering to a patient in need thereof a therapeutically effective amount of a pharmaceutical composition comprising a solid dispersion and one or more pharmaceutically acceptable excipients, wherein the solid dispersion comprises compound (1) as defined below or a pharmaceutically acceptable salt thereof 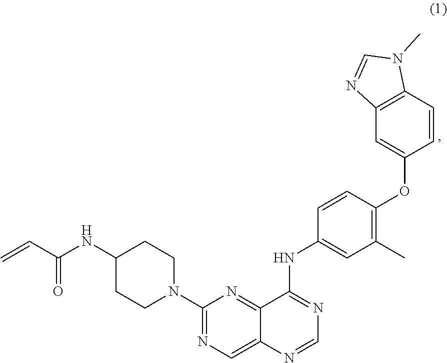 [SMILES: C=CC(=O)NC1CCN(c2ncc3ncnc(Nc4ccc(Oc5ccc6c(c5)ncn6C)c(C)c4)c3n2)CC1]
wherein compound (1) or the pharmaceutically acceptable salt thereof is administered following administration of a systemic anti-cancer therapy agent.
